(19) United States
(12) Patent Application Publication
(10) Pub. No.: US 2022/0380410 A1
(43) Pub. Date: Dec. 1, 2022

(54) Live Attenuated Universal Influenza Virus Vaccines, Methods and Uses Thereof

(71) Applicant: Pentavalent Bio Sciences Pvt Ltd, Bangalore (IN)

(72) Inventors: Bhavani Venkataswamachari Peddayelachagiri, Bangalore (IN); Soumya Paul, Bangalore (IN)

(21) Appl. No.: 17/616,137

(22) PCT Filed: Jun. 2, 2020
(86) PCT No.: PCT/IB2020/055192

§ 371 Date: Dec. 2, 2021

(30) Foreign Application Priority Data
Jun. 2, 2019 (IN) 201941008237

(51) Int. Cl. C07K 14/005; C12N 7/00; A61K 39/145; A61P 31/16

(52) U.S. Cl. CPC C07K 14/005; C12N 7/00; A61K 39/145; A61P 31/16; C12N 2760/16122; C12N 2760/16121; C12N 2760/16171; C12N 2760/16134; A61K 2039/5254

(57) ABSTRACT

The present invention provides a modified influenza viruses comprising haemagglutinin and a headless haemagglutinin. The haemagglutinin is provided by a source exogenous to the virus and the headless haemagglutinin is encoded by the viral genome. The present disclosure also provides modified influenza viruses comprising a headless haemagglutinin. The present disclosure also provides vaccine compositions comprising the modified influenza viruses. The vaccine compositions of the present disclosure can elicit broad neutralizing antibodies and provide cross-protection across various influenza strains. Methods, compositions and cells for propagating the modified influenza viral strains related to vaccines is also provided.

### CROSS-REFERENCE TO RELATED APPLICATIONS

This application is a 371 of International Application No. PCT/IB2020/055192, filed Jun. 2, 2020, which application claims priority to India Application Serial No. 201941008237, filed Jun. 2, 2019, which applications are incorporated herein by reference in their entirety.

### INCORPORATION BY REFERENCE OF SEQUENCE LISTING PROVIDED AS A TEXT FILE

A Sequence Listing is provided herewith in a text file, MAJU-001_SEQ_LIST_(rev Jun 2022)_ST25, created on Jun. 17, 2022 and having a size of 173,000 bytes of file. The contents of the text file are incorporated herein by reference in its entirety.

### FIELD OF THE INVENTION

The present invention relates to viral vaccines. More specifically the present invention relates to genetically engineered live attenuated influenza virus vaccines.

### BACKGROUND AND THE PRIOR ART

Vaccination is the best effective way to provide protection against influenza virus infection. Due to rapid antigenic variations: every 3 to 5 years Influenza virus strains get replaced by new variant and that manages to evade existing antibody responses. Due to lack of immunity against the newly circulating strains of Influenza, the cumulative morbidity and mortality is relatively high.

Isolates required for vaccine preparation must be selected every year as per surveillance report generated by World Health Organization (WHO) collaborating centers. Often the predictions are inaccurate, resulting into substantial drop in the efficacy of vaccination.

Live, attenuated vaccines are well known in the art where the disease-causing virus is passed through a series of cell cultures or animal embryos (typically chick embryos). With each passage, the virus becomes better at replicating in cell cultures, but loses its ability to replicate in human cells. All of the methods that involve passing a virus through a non-human host produce a version of the virus that can still be recognized by the human immune system, but cannot replicate well in a human host. Protection from a live, attenuated vaccine typically outlasts that provided by a killed or inactivated vaccine.

Virus like particles based vaccine can also be formed by genetic modifications such as in Influenza Virus Vaccine Based on the Conserved Haemagglutinin Stalk Domain, by John Steel, et. al, mbi.asm.org, April 2010, Vol 1, Issue 1. It discloses novel immunogen comprising the conserved influenza HA stalk domain and lacking the globular head. The approach is based on presentation to the host immune system of a region of the influenza virus called a “headless haemagglutinin” (headless HA) which is similar among a multitude of diverse strains. The study showed that vaccination of mice with a headless HA confers protection to animals against a lethal influenza virus challenge. Through further development and testing, the study predicts that a single immunization with a headless HA vaccine will offer effective protection through several influenza epidemics.

US2010297174 discloses one such influenza haemagglutinin stem domain polypeptides, compositions comprising the same, vaccines comprising the same and methods of their use. It teaches that the globular head domain of an influenza haemagglutinin comprises one or more highly immunogenic regions. These highly immunogenic regions might generate a host immune response. However, the highly immunogenic regions might also vary from strain to strain of influenza virus.

Further Reversion of Cold-Adapted Live Attenuated Influenza Vaccine into a Pathogenic Virus, by Bin Zhou, et al, Journal of Virology, October 2016, Volume 90, Number 19, discloses live attenuated influenza vaccine FluMist. It discloses that Influenza A viruses are encoded by eight segments of negative sense RNA (viral RNA [vRNA]), and FluMist LAIVs are reassortant viruses that contain the HA and NA surface glycoprotein vRNAs from contemporary strains and the remaining six backbone vRNA segments (PB2, PB1, PA, NP, M, and NS) from a cold-adapted master donor virus. HA and NA are the major antigenic determinants of FluMist and are from strains biannually predicted by a WHO vaccine strain recommendation committee to provide the best antigenic match to the strains circulating in humans.

The cold adapted live attenuated vaccine strain of influenza, compared to inactivated or subunit vaccine preparations, has provided the most effective and broad range protection against flu due to the degree of antigenic similarity with the circulating strains. The elderly persons still fail to evoke sufficient immune responses in view of low stability often combined with low expression rate of foreign proteins in influenza virus against influenza infection. This is one of the most important drawbacks in present influenza vaccine strategy and still there is high demand for universal vaccine inducing cellular and humoral immune response with stably expressing high amounts of viral proteins.

Influenza is a historical respiratory disease with global importance with respect to human health and economy. It occurs as seasonal epidemics during winter months and global pandemics which can occur during any season and last more than a year. The World Health Organization estimation reveals that about 3-5 million severe illness cases worldwide resulting in about 250,000 to 500,000 deaths annually.

Vaccination is the best effective way to provide protection against influenza virus infection. The influenza vaccines come in three major formulations: (1) inactivated whole-virus, (2) “detergent”-split, and (3) subunit vaccines. The most widely used seasonal influenza vaccine is the trivalent inactivated vaccine (TIV) usually composed of two influenza A virus types and a B type. TIVs provide immunity by inducing antibodies that target the protective epitopes on HA. Some formulations may also induce NA specific antibodies that do not protect from infection but may modulate the resulting disease. Almost all multinational vaccine formulations are egg-based. However, FDA has discouraged the use of egg-based vaccines, due to adverse reactions from egg-based proteins, particularly in children. Safe and controlled mammalian cell culture technology can be implemented as an intervention to this challenge. The key benefit of cell culture vaccine is its potential to scale up and produce large quantities quickly as required; it also has a much more sterile and faster production cycle, without the external dependence on eggs and thus enabling quicker response times in the event of a pandemic. Ideally, a live attenuated vaccine is considered to be safe and effective in providing protection, and cost-effective in production. Attenuation of the vaccine is required to prevent clinical symptoms upon administration, at high dose too. Such vaccine should be immunogenic so as to confer specific and promising protection against infection. Previous efforts to provide a safe, live attenuated influenza vaccine have focused on cold-adapted influenza viruses, chimeric influenza viruses (Muster et al., 1992), influenza viruses lacking functional or complete haemagglutinin, influenza viruses comprising mutant matrix protein. Since timely and sufficient supply of vaccine against a new emerging pandemic strain is inadequate in every country around the world. Also, due to rapid antigenic variations, every year Influenza virus strains get replaced by new variant and that manages to evade existing immune responses in the host. Thus, annual vaccination against circulating seasonal influenza strains is the current preventive strategy in practice.

The selection of influenza strains to prepare annual vaccines is based upon the global surveillance of influenza viruses in circulation and the spread of new strains around the world. World Health Organization (WHO) makes recommendations on updating the influenza vaccines (which includes three classes of licensed vaccines namely inactivated viruses, live attenuated virus and recombinant haemagglutinin vaccines) for every next seasonal vaccination based on the global surveillance report. Often the predictions are inaccurate, resulting into substantial drop in the efficacy of vaccination. The elderly persons still fail to evoke sufficient immune responses in view of low stability often combined with low expression rate of foreign proteins in influenza virus against influenza infection. This is one of the most important drawbacks in present influenza vaccine strategy and still there is a pressing demand for universal vaccine inducing cellular and humoral immune response.

There are various live attenuated influenza vaccine candidates that are developed and characterized for heterosubtypic protective response, including influenza preparations attenuated by cold adaptation, NS deletions, HA cleavage site mutations, packaging site changes, gamma irradiation, non-replicating adenoviruses capable of expressing influenza NP and M2 proteins and inactivation of HA signal sequence along with HA cleavage site. Of these, influenza preparations attenuated by cold adaptation, NS deletions, HA cleavage site mutations, packaging site changes, gamma irradiation and inactivation of HA signal sequence along with HA cleavage site induce heterotypic immunity with doses of ˜10⁶ or less influenza infectious units and non-replicating adenoviruses require a dose of ˜1×10¹⁰ particles of each recombinant adenovirus to induce protective response. In the adenovirus experiment immunization with either NP or M2 separately resulted in significant weight loss after challenge compared to the combination. Notably, live attenuated influenza-based vaccine candidates may have properties like expression of all of the core proteins in the cytosol to induce efficient antigen presentation and thus a broader repertoire of T cells. Also, they will introduce influenza RNA into the endosome and cytosol to initiate an innate response, which could promote a favourable adjuvant effect. Influenza pseudotyped virus attenuated by inactivation of HA signal sequence along with HA cleavage site is a suitable example in this case. This vaccine induced heterotypic immunity with doses of ˜10⁶ but it failed to induce neutralizing antibodies at lesser doses when administered intranasally. It required a 10-fold increased dosage via intraperitoneal route to induce only strain-specific neutralizing antibody. This pseudotyped influenza virus could successfully infect cells and express the viral core proteins and neuraminidase but cannot replicate. Replication defective viruses, though elicits protective immunity, fail to replicate the viral genome and, in consequence, do not synthesize significant quantities of viral proteins, many of which are known to elicit protective immune responses and also viral RNA that are capable of imparting adjuvant effect. An alternative approach is to make use of a virus that is replication-competent but produces non-infectious particles by virtue of the absence of a single essential virion protein. A single-cycle virus with these characteristics should present all of the virus-specific proteins, apart from the absent one, to all arms of the immune response. In the present invention, replication-competent and replication-incompetent viruses have been developed keeping all these concerns and crucial aspects in view.

The present invention therefore overcomes the challenges of the prior art and provides effective techniques wherein live attenuated influenza viruses three major glycoproteins namely haemagglutinin, neuraminidase and a headless haemagglutinin possessing only the stalk portion with or without globular domain lacking the receptor binding site of the protein on its envelope are developed with possibility of using as universal influenza vaccine to confer protection against a broad range of influenza strains including any existing and/or emerging ones.

### OBJECT OF THE INVENTION

It is an object of the invention to overcome the drawbacks of the prior art.

It is the principal object of the present invention to generate live attenuated strains of influenza belonging to type A groups

It is another object to provide composition and method to prepare a genetically engineered universal influenza vaccine.

It is yet another object of the invention to provide a method of developing single cycle replication-competent live attenuated influenza strain as vaccine strains or immunogenic composition.

It is a further object of the present invention to generate a modified mammalian cell line in which the live attenuated strains of influenza shall replicate continuously.

It is another object of the present invention to generate the live attenuated influenza strains so as to replicate continuously only in the modified cell line and replicate only once (single cycle replicative) in any other mammalian cell line in vitro or mammalian cells in vivo.

It is another object of the present invention to express a headless haemagglutinin on the surface of the live attenuated strains of influenza as a modified glycoprotein.

It is yet another object of the present invention to use attenuated influenza strains as vaccine in a combination that confer broad range of cross protection to a host against infection from any existing or emerging influenza strains belonging to type A group of influenza.

It is a further object of the present invention to use live attenuated influenza strains as single cycle replicative vaccine for administration in healthy adults so as to mimic wild virus infection and induce influenza-specific immune response against infection from pan influenza strains.

It is another object of the present invention to further modify live attenuated influenza strains in vitro so as to make them replication-incompetent and use the them as vaccine that confers broad range of immune response and cross-protection against any existing or emerging strains of influenza in high risk group of individuals including new born children, pregnant women, immune compromised individuals, elderly people (above 65 years of age) and others.

It is a further object of the present invention to produce the live attenuated influenza strains in bulk for high yield of purified virus strains.

It is yet another object of the present invention to evaluate safety of the vaccine formulation in mice model.

It is a further object of the present invention to immunogenicity of the vaccine formulation in mice model.

It is another object of the present invention to provide live attenuated vaccine strains of influenza which provides broader cross protection.

It is yet another object of the invention to provide a replication-incompetent live attenuated influenza strain as vaccine strains or immunogenic composition.

It is a further object of the present invention to provide a replication-incompetent live attenuated vaccine strain for high risk group including persons 65 years of age and older, children younger than 2 years of age, pregnant women, and people of any age who have certain medical conditions, such as asthma, chronic heart, lung, kidney, liver, blood, or metabolic diseases like diabetes, compromised immune systems, or morbid obesity from both pandemic and seasonal influenza.

### SUMMARY OF THE INVENTION

According to an aspect of the present invention, there is provided a modified influenza virus encoding vRNA of conserved stalk domain containing headless HA and complete haemagglutinin protein being provided by complementary cell line in Trans.

According to another aspect of the present invention, there is provided a modified influenza virus comprising a headless haemagglutinin.

According to yet another aspect of the present invention there is provided a vaccine composition comprising the modified influenza virus.

According to yet another aspect of the present invention, there is provided a method of immunizing a subject, comprising administering to the subject the vaccine composition.

According to yet another aspect of the present invention, there is provided a method of producing the modified virus comprising: transfecting a viral RNA encoding the headless haemagglutinin into a cell line stably expressing the haemagglutinin and harvesting the modified virus.

According to yet another aspect of the present invention, there is provided a method of producing the modified virus comprising: transfecting a viral RNA encoding the headless haemagglutinin into a cell line stably expressing the haemagglutinin; harvesting the modified virus comprising the haemagglutinin and the headless haemagglutinin from cell supernatant; infecting a mammalian cell line with the modified virus comprising the haemagglutinin and the headless haemagglutinin; and harvesting the modified virus comprising the headless haemagglutinin from cell supernatant.

According to still an aspect of the present invention, there is provided a method of treatment or prevention of infection by influenza comprising administering immunologically effective amount of a virus to the human or non-human animal.

According to still an aspect of the present invention, there is provided a kit comprising the immunogenic composition comprising the modified influenza virus, and adjuvants and/or diluents and an instruction leaflet relating to administration.

According to still an aspect of the present invention, there is provided an immunogenic composition comprising the modified influenza virus.

According to still an aspect of the present invention, there is provided use of a modified influenza virus for the manufacture of a medicament comprising vaccine for the protection of human or non-human animals against infection with a pathogenic virus or the pathogenic effects of infection.

According to still an aspect of the present invention, there is provided use of headless haemagglutinin glycoprotein (stem/stalk region of hemagglutinin) in inducing cell mediated and humoral immune response in mice model

According to still an aspect of the present invention, there is provided use of headless haemagglutinin glycoprotein (stem/stalk region of hemagglutinin) in imparting broad range of influenza specific immune response in mice model

According to a further aspect of the present invention, there is provided a method of generating genetically modified cell line in vitro comprising transforming the cell line with modified strain of claim 1, such that the attenuated influenza strain replicates constitutively

According to a further aspect of the present invention, there is provided a modified mammalian cell line wherein attenuated influenza vaccine strain replicates continuously.

According to still an aspect of the present invention, there is provided a modified influenza virus strain encoding atleast three glycoproteins comprising
a. functional or complete haemagglutinin (fHA or HA) having sequence SEQ ID 2 or 35 or 37 with the variable globular head and the conserved stalk region;
b. truncated or headless haemagglutinin (tHA or ΔHA) having sequence SEQ ID 3 or 4 or 19 through 34 having only conserved stalk region and
c. functional neuraminidase (NA)-having sequence SEQ ID 5 or 6 or 39 through 42.

According to still an aspect of the present invention, there is provided a polynucleotide sequence having headless haemagglutinin (tHA or ΔHA) with only conserved stalk region with or without globular domain lacking receptor binding site of SEQ ID 3 or 4 or 19 through 34.

According to still an aspect of the present invention, there is provided a polypeptide comprising the amino acid sequence of SEQ ID NO: 4, 18, 20, 22, 24, 26, 28, 30, 32 or 34.

### BRIEF DESCRIPTION OF ACCOMPANYING DRAWINGS

FIG. 1 illustrates strategy of developing replication-competent and replication incompetent influenza viruses.

FIG. 2 illustrates immunostaining of HEK/HA cells with monoclonal anti-human Influenza A (H1N1, H2N2) (Clone C179) reveals the expression of functional or complete haemagglutinin and its proper folding and packaging in cell surface.

FIG. 3 illustrates microscopic observation of HEK/HA cells infected with replication-competent influenza virus for cytopathic effect.

FIG. 4 illustrates dot blot assay of both PFV and PFV-S variants of influenza virus strains showing reactivity with C179 and CR6261 antibodies indicating the properly folding of AHA on their envelope.

FIG. 5 illustrates virus spread assay on (A) MDCK and (B) MDCK/HA cells infected with PFV at an MOI of 0.001 for 72 h and stained for intracellular nucleoprotein.

FIG. 6 illustrates growth kinetics of PFV and PFV-S in MDCK/HA and MDCK cells. MDCK/HA and MDCK cells were infected with the PFV at an MOI of 0.001. At the indicated times after infection, the virus titer in the supernatant was determined by (A) RT-PCR and (B) focus forming assay. The values presented are means from duplicate experiments. PR8 strain of influenza was used as the wild type virus for reference.

FIG. 7 illustrates induction of virus-specific IgG and IgA in serum of mice immunized with PFV and PFV-S. Virus-specific antibodies were detected by means of an ELISA. Samples from six mice from each group were obtained 4 weeks after the final vaccination. Results are expressed as the mean absorbance (±standard deviations) of 1:10 diluted samples.

FIG. 8 illustrates cytokine production in the serum post immunization. Cytokine concentrations in mice sera were determined using mouse cytokine group I panel 8-plex kit. The following antibodies were used in the ELISAs: anti-IL-1β, anti-IL-2, anti-IL-4, anti-IL-5, anti-IL-10, anti-GM-CSF, anti-IFNγ and anti-TNFα. The concentration of IL-2 cytokine in all mice sera samples was below detection limit. Data shown are means±SD for all groups of 2 mice per group.

FIG. 9 illustrates body weight changes for immunized mice challenged with wild-type viruses including (A) A/PR/8/34 (H1N1) and (B) A/HKx31 (H3N2). Mice challenged with 32 HAU of mouse adapted wild virus (8 mice per group) 6 weeks after the final vaccination were observed for both weights and survival rate was monitored for 14 days after challenge.

### DETAILED DESCRIPTION OF THE PRESENT INVENTION

The present invention provides live attenuated vaccine strains of influenza developed using a novel technology so that it expresses a highly conserved haemagglutinin within its envelope in two formats: (a) complete HA trimer with the stalk and globular head (fHA or HA) and (b) the stalk portion only (tHA or ΔHA) along with another surface glycoprotein known as neuraminidase, matrix protein, nucleoprotein and all other necessary proteins present in wild strain. Thus, the live attenuated influenza vaccine strain has three glycoproteins on its envelope namely, HA, AHA and NA unlike any wild influenza strain which has only HA and NA. The present inventors have surprisingly found that incorporating AHA in whole virus form of influenza virus strain and using the same as vaccine ensures that the live attenuated influenza virus strain can never revert back to its wild type. The virus can replicate only once in any other unmodified in vitro or in vivo cells or cell lines. The virus can continuously replicate in modified cell line which constitutively expresses complete HA sequence.

Approaches to developing such stalk-focused universal vaccines have included headless HA, recombinant soluble HA, synthetic polypeptides, prime-boost regimens, nanoparticles, and recombinant influenza viruses expressing chimeric HA. Influenza headless haemagglutinin polypeptides comprising of highly conserved stalk domain lacking all or substantially all of globular domain have been earlier evaluated as for immunogenic compositions (e.g. Vaccines) capable of generating immune responses against a plurality of influenza virus strains. The rationale of generating such a polypeptide by removing the highly immunogenic globular domain and retaining only stalk domain of haemagglutinin was to advantageously allow a broad range of immune response against the one or more less immunogenic epitopes of the stem domain polypeptide to develop. The typical primary structure of this influenza hemagglutinin stem domain polypeptide comprises, in the following order, a signal peptide, an HA1 N-terminal stem segment, a linker, an HA1 C-terminal stem segment and an HA2. Vaccination of mice with this headless HA polypeptide-based immunogen elicited immune sera with broader reactivity and thereby provided full protection against death and partial protection against disease following lethal homologous viral challenge. But, till date no influenza virus with such stalk domain containing headless HA on virus envelope and vRNA encoding for the same as glycoprotein has been generated either as replication-competent or replication-incompetent variants for use as an immunogenic composition like vaccines and/or therapeutics.

The present invention relates to the development of modified live attenuated influenza virus strains to display a modified haemagglutinin, representing the highly conserved stalk domain with or without the portion of globular domain that is highly conserved to generate protective antibodies, on their surface, and compositions comprising the modified live attenuated strains in eliciting broad spectrum protective immune response against influenza virus infection, vaccines comprising the same and methods of their use.

The said live attenuated influenza virus according to the invention is also modified so that the vRNA encoding for haemagglutinin has been modified such that the resultant glycoprotein lacks the receptor binding region in its globular domain and encodes for the highly conserved stalk portion of haemagglutinin. The modified RNA retains all other properties required for viral packaging and expression of headless haemagglutinin protein.

With respect to the use of any plural and/or singular terms herein, those having skill in the art can translate from the plural to the singular and/or from the singular to the plural as is appropriate to the context and/or application. The various singular/plural permutations may be expressly set forth herein for sake of clarity. The use of the expression “at least” or “at least one” suggests the use of one or more elements or ingredients or quantities, as the use may be in the embodiment of the disclosure to achieve one or more of the desired objects or results. Throughout this specification, the word “comprise”, or variations such as “comprises” or “comprising” or “containing” or “has” or “having” wherever used, will be understood to imply the inclusion of a stated element, integer or step, or group of elements, integers or steps, but not the exclusion of any other element, integer or step, or group of elements, integers or steps.

Reference throughout this specification to “one embodiment” or “some embodiments” means that a particular feature, structure or characteristic described in connection with the embodiment may be included in at least one embodiment of the present disclosure. Thus, the appearances of the phrases “in one embodiment” or “in some embodiments” in various places throughout this specification may not necessarily all refer to the same embodiment. It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination.

The term “influenza virus” or “influenza virion” as used herein refers to the infectious form of influenza virus outside a host cell, with a core of viral RNA enclosed in a capsid which is in turn enclosed in a lipid bilayer expressing surface glycoproteins. Major surface glycoprotein of the influenza virus includes haemagglutinin (HA) and neuraminidase (NA).

The term “single-cycle replication competent virus” as used herein refers to a virus that infects a host cell and undergoes only one cycle of replication to produce progeny virus. The progeny virus generated from the single-cycle replication competent virus cannot replicate in the host cell.

The term “replication incompetent virus” as used herein refers to a virus that cannot bind to a host cell and cannot replicate in the host cell.

The term “haemagglutinin” or “HA” as used herein refers to influenza haemagglutinin and includes 18 sub-types of influenza A haemagglutinin (H1 to H18). Throughout this disclosure, the term “haemagglutinin” is used to refer to the full-length or wild-type or fully functional haemagglutinin whereas the term “headless haemagglutinin” is used to refer to haemagglutinin that lacks the receptor binding region of the HA1 domain of the haemagglutinin.

The term “receptor binding region of haemagglutinin” as used herein refers to that region of the influenza haemagglutinin that binds to a sialic acid receptor on host cells. The receptor binding region of the haemagglutinin is present within an HA-1 domain of the haemagglutinin.

In the present invention, by controlling the haemagglutinin provided exogenously and the headless haemagglutinin expressed by the modified virus, the nature of the immune response generated on exposure to the modified virus can be controlled. Furthermore, by carefully selecting the haemagglutinin exogenously provided and present on the modified virus' surface, the interaction of the modified virus with a host airway can be controlled.

The headless haemagglutinin present in any of the modified influenza viruses of the present invention is encoded by the modified virus' genome. The viral genome, i.e., the viral RNA, is modified to remove the RNA sequence encoding the sialic acid receptor binding region of the haemagglutinin thereby providing a headless haemagglutinin lacking the sialic acid receptor binding region. The HA1 domain of the wild-type haemagglutinin comprises the sialic acid receptor binding region.

In some embodiments, the headless haemagglutinin lacks a sequence from amino acids 58 to 295 of the influenza haemagglutinin. In some embodiments, the headless haemagglutinin lacks amino acids 60-291, 69-293, 108-242, or 114-244 of the influenza haemagglutinin. In some embodiments, the headless haemagglutinin is the H1 sub-type of influenza A haemagglutinin that lacks amino acids 60-291 or 108-242. In some embodiments, the headless haemagglutinin is the H3 sub-type of influenza A haemagglutinin that lacks amino acids 69-293 or 114-244.

In some embodiments, the headless haemagglutinin comprises an N-terminal region of the HA1 domain covalently linked to a linker which is in turn covalently linked to a C-terminal region of the HA1 domain which is in turn covalently linked to the HA2 domain. In some embodiments, the headless haemagglutinin comprises an N-terminal region of the HA1 domain covalently linked, without a linker, to a C-terminal region of the HA1 domain which is in turn covalently linked to the HA2 domain.

In some embodiments, the N-terminal region of the HA1 domain present in the headless haemagglutinin comprises amino acids 1-59, 1-68, 1-107, or 1-113 of the influenza haemagglutinin. In some embodiments, the N-terminal region of the HA1 domain present in the headless haemagglutinin comprises amino acids 1-59 or 1-107 of the H1 sub-type of influenza haemagglutinin. In some embodiments, the N-terminal region of the HA1 domain present in the headless haemagglutinin comprises amino acids 1-68 or 1-113 of the H3-sub-type of influenza haemagglutinin.

In some embodiments, the C-terminal region of the HA1 domain present in the headless haemagglutinin comprises amino acids 292-343, 243-343, 294-344, or 245-344 of the influenza haemagglutinin. In some embodiments, the C-terminal region of the HA1 domain present in the headless haemagglutinin comprises amino acids 292-343 or 243-343 of the H1 sub-type of influenza haemagglutinin. In some embodiments, the C-terminal region of the HA1 domain present in the headless haemagglutinin comprises amino acids 294-344 or 245-344 of the H3 sub-type of influenza haemagglutinin.

In some embodiments, the HA2 domain present in the headless haemagglutinin comprises amino acids 344-566 or 345-566 of the influenza haemagglutinin. In some embodiments, the HA2 domain present in the headless haemagglutinin comprises amino acids 344-566 of the H1 sub-type of influenza haemagglutinin. In some embodiments, the HA2 domain present in the headless haemagglutinin comprises amino acids 345-566 of the H3 sub-type of influenza haemagglutinin.

In some embodiments, the linker present in the headless haemagglutinin is a peptide linker. In some embodiments, the linker comprises about 1-50, about 1-40, about 1-30, about 1-25, about 1-20, about 1-15, about 1-10, or about 1-5 amino acids. In some embodiments, the linker comprises about 3-8 glycine residues and a serine reside. In some embodiments, the linker is selected from the group consisting of Gly, Gly-Gly, Gly-Gly-Gly, Gly-Gly-Gly-Ser, Gly-Gly-Gly-Gly, Gly-Gly-Gly-Gly-Ser, Gly-Gly-Gly-Gly-Gly, and Gly-Gly-Gly-Gly-Gly-Ser. In some embodiments, the linker has a formula (G)ₙ-S, where G stands for glycine, S stands for serine, and n is 2-6. In some embodiments, the headless haemagglutinin comprises 1-5 repeats of the linker (G)ₙ-S.

Sequence IDs listed in the present invention are as follows:
a. Sequence ID No. 3, 4, 5, 6, 19 to 34 represents headless haemagglutinin with or without globular domain lacking receptor binding site (AHA) variants.
b. Sequence ID 1, 2, 35, 36, 27 and 38 represent complete or functional or complete haemagglutinin (HA).
c. Sequence ID 5, 6, 39, 40, 41 and 42 represent neuraminidase (NA).
d. Sequence ID 7, 8, 43 and 44 represent polymerase basic protein 1 (PB1).
e. Sequence ID 9, 10, 45 and 46 represent polymerase basic protein (PB2).
f. Sequence ID 11, 12, 47 and 48 represent polymerase acidic protein (PA).
g. Sequence ID 13, 14, 49 and 50 represent nucleoprotein (NP).
h. Sequence ID 15, 16, 51 and 52 represent matrix protein (M).
i. Sequence ID 17, 18, 53 and 54 represent non-structural protein (NS).

In some embodiments, the headless haemagglutinin comprises a sequence selected from the group consisting of SEQ ID Nos.: 1, 18, 20, 22, 24, 26, 28 and 30. In some embodiments, the viral RNA of the modified influenza virus of the present invention comprises a sequence encoding a headless haemagglutinin selected from the group consisting of SEQ ID Nos.: 3, 19, 21, 23, 25, 27, 29 and 31.

In some embodiments, the modified influenza virus comprises haemagglutinin selected from 18 sub-types of influenza A haemagglutinin (H1 to H18) and a headless haemagglutinin that a sequence from amino acids 58 to 295 of haemagglutinin. In some embodiments, the modified influenza virus comprises haemagglutinin selected from 18 sub-types of influenza A haemagglutinin (H1 to H18), and a headless haemagglutinin that lacks amino acids 60-291, 69-293, 108-242, or 114-244 of haemagglutinin. In some embodiments, the modified influenza virus comprises haemagglutinin selected from 18 sub-types of influenza A haemagglutinin (H1 to H18) and a headless haemagglutinin that is the H1 sub-type of influenza A haemagglutinin lacking amino acids 60-291 or 108-242. In some embodiments, the modified influenza virus comprises haemagglutinin selected from 18 sub-types of influenza A haemagglutinin (H1 to H18) and a headless haemagglutinin that is the H3 sub-type of influenza A haemagglutinin lacking amino acids 69-293 or 114-244.

In some embodiments, a viral RNA sequence encoding a truncated haemagglutinin comprises an untranslated region (UTR) at the 5′ and the 3′ end. That is in these embodiments, the engineered influenza virus of the present invention comprises a viral RNA having the sequence: 5′UTR-a sequence encoding a truncated haemagglutinin-3′UTR.

One of the concerns associated with using live attenuated viruses containing a haemagglutinin gene from a potentially pandemic strain is that, in rare cases of co-infection of a host with a live attenuated virus and a seasonal viral strain, a reassortment of genes may result such that the haemagglutinin gene from the live attenuated virus could become incorporated into the seasonal strain to form a new epidemic or pandemic strain. The modified influenza viruses of the present invention are not associated with such risks. The loss of the receptor binding region of the globular domain from the expressed headless haemagglutinin protein means that even if the genes is reassorted into another strain it would result in a strain that will be unable to replicate due to a lack of functional or complete haemagglutinin on the viral particle surface. Also, the RNA segments of the viral genome, except for the headless HA and NA, are selected from the mouse adapted strain of influenza i.e., A/PR/8/34 (H1N1). Reassortment of these genes into another wild strain would result in a recombinant strain that will be unstable and or unable to replicate in the human and non-human animal.

The modified influenza virus of the present invention is a live attenuated virus. The modified influenza virus of the present invention comprises a viral RNA encoding the headless haemagglutinin as described above and further comprises viral RNAs encoding other influenza proteins selected form the group consisting of: neuraminidase, M2 protein, M1 matric protein, nucleocapsid protein, PB1, PB2, and PA.

Also provided herein are methods for producing the engineered or modified influenza virus of the present invention. In some embodiments, the engineered or modified influenza virus comprising haemagglutinin and a headless haemagglutinin is produced by transfecting a viral RNA encoding the headless haemagglutinin and other influenza viral proteins into a cell line stably expressing haemagglutinin. The viral RNA replicates in the cell line thereby producing the headless haemagglutinin and other viral proteins. The headless haemagglutinin and the haemagglutinin produced by the cell line get packaged along with other viral proteins and bud from the cell membrane to produce the engineered virus comprising haemagglutinin and the headless haemagglutinin. The engineered virus is subsequently harvested from the cell supernatant.

In some embodiments, the engineered or modified influenza virus comprising a headless haemagglutinin is produced by transfecting a viral RNA encoding the headless haemagglutinin and other influenza viral proteins into a cell line stably expressing haemagglutinin; harvesting the engineered virus particles comprising the haemagglutinin and the headless haemagglutinin; infecting a mammalian cell line with the engineered virus comprising the haemagglutinin and the headless haemagglutinin; and harvesting the engineered influenza virus comprising the headless haemagglutinin from the cell supernatant.

The present disclosure also provides cell lines stably expressing influenza haemagglutinin. In some embodiments, the cell line is an MDCK cell line or an HEK293T cell line stably expressing haemagglutinin of H1 to H18 sub-type of influenza A. In one embodiment, the cell line stably expressing influenza haemagglutinin is an HEK293T cell line having ATCC Deposit No. PTA-125121.

The live attenuated vaccine strain that originates from unmodified cells is without HA and cannot replicate or infect any other cells. The live attenuated influenza strain of the present invention is designed to have a headless heamagglutinin glycoprotein lacking receptor binding region of globular domain on its surface along with the two glycoproteins HA and NA. This novel glycoprotein incorporated being the conserved HA stalk protein hereafter termed as “headless HA (AHA)” that is intended to confer broad range of protection against influenza caused by any influenza strain belonging to Type A and Type B influenza viruses.

The said live attenuated influenza virus according to the invention is also modified so that the vRNA encoding for haemagglutinin has been modified such that the resultant glycoprotein lacks the receptor binding region in its globular domain and encodes for the highly conserved stalk portion of haemagglutinin. The modified RNA retains all other properties required for viral packaging and expression of headless haemagglutinin protein.

The present invention provides live attenuated vaccine strains of influenza developed using a novel technology so that it expresses a highly conserved hemagglutinin (HA) within its envelope in two formats: (a) complete HA trimer with the stalk and globular head (fHA) and (b) the stalk portion only (AHA) along with another surface glycoprotein known as neuraminidase, matrix protein, nucleoprotein and all other necessary proteins present in wild strain. Thus, the live attenuated influenza vaccine strain has three glycoproteins on its envelope namely, HA, AHA and NA unlike any wild influenza strain which has only HA and NA. The present inventors have found that incorporating AHA in whole virus form of influenza virus strain and using the same as vaccine ensures that the live attenuated influenza virus strain can never revert back to its wild type. The virus can replicate only once in any other unmodified invitro or invivo cells or cell lines. The virus can continuously replicate in modified cell line which constitutively expresses complete HA sequence.

In accordance with the invention, modified influenza virus strain encoding atleast three glycoproteins comprising

functional or complete haemagglutinin (fHA or HA) having sequence SEQ ID 2 or 35 or 37 with the variable globular head and the conserved stalk region;

truncated or headless haemagglutinin (tHA or ΔHA) having sequence SEQ ID 3 or 4 or 19 through 34 having only conserved stalk region and

functional neuraminidase (NA)-having sequence SEQ ID 5 or 6 or 39 through 42.

The live attenuated influenza strains of the present invention include a replication-competent virus with three major glycoproteins (HA, NA and AHA) on its envelope. Here, AHA represents headless haemagglutinin of A/Michigan/45/2015 (H1N1) consists of the conserved stalk domain along HA1 domain devoid of the globular domain and it is designed to enable increased access of HA stalk to immune system so as to induce broad range of immune response against type A influenza virus. The same strategy can be elaborated to type B influenza viruses too wherein, AHA from any suitable Type B virus can be designed to incorporate on the replication-competent virus envelope. Second attenuated influenza strain of the present invention is a replication-incompetent virus with two glycoproteins (NA and ΔHA) on its envelope, similar to that of replication-competent virus and does not have complete HA.

In accordance with the invention, replication-competent vaccine strains can be made rapidly by standardization of production protocol, and incorporate any HA, NA and ΔHA in the envelope. They are self-replicating in vitro only the modified stable cell lines which constitutively expresses complete functional HA in glycoprotein form. And thus, the modified stable cell lines are integral component for bulk production of the vaccine. Along with ΔHA, HA and NA, matrix protein (M) and nucleoprotein (NP) are also present in this vaccine strain in order to collectively elicit broadly protective antibodies by triggering both adaptive and innate immunity against flu infection. Replication-competent vaccine strain is based on trimmed vRNA encoding HA, so does not contain a viable HA vRNA that could reassort with any other influenza strain. But, as it has fully functional HA as only glycoprotein on its envelope, which enables the virus to bind to the host cell receptor and complete virus replication of one cycle. In order to strictly control reassortment of this vaccine strain with wild virus in human and non-human primates, the virus backbone also has been adopted from mouse adapted PR8 strain. Upon administration to host as vaccine, the replication-competent virus can infect, replicate once resulting in antigen amplification within the host and induce broadly reactive strong cellular and humoral immune responses following a rigorously controlled single cycle of replication and infection in the lung. Due to the antigen amplification property of the replication-competent vaccine strain, ideally very less dose would be required for administration and thus more doses of vaccine can be produced in lesser production volumes thus enabling cost effective bulk production of live attenuated vaccine.

In accordance with the invention, replication-incompetent vaccine strains can also be made rapidly by standardization of production protocol, and incorporate any NA and ΔHA in the envelope. They are produced by in vitro replication of replication-competent virus in any suitable naïve cell line. This vaccine strain mimics wild virus in structure and has matrix protein (M) and nucleoprotein (NP) along with HA and NA. Thus this vaccine strain also is capable of inducing broadly reactive strong cellular and humoral immune responses following orchestrated antigen processing by the host immune system. Replication-incompetent vaccine strain is based on trimmed vRNA encoding HA, so does not contain a viable HA vRNA that could reassort with any other influenza strain. In order to strictly control reassortment of this vaccine strain with wild virus in human and non-human primates, the virus backbone also has been adopted from mouse adapted PR8 strain. Unlike replication-competent vaccine strain, this attenuated virus cannot replicate, infect or result antigen amplification within the host.

The vaccine strain according to the present invention is designed to mimic the wild flu viral strain in terms of the exact structure and the same antigens, with additional headless hemagglutinin without globular head to provide broad range protection; yet non-infective with one cycle replication capability similar to their respective wild strains. Thus the vaccine strains are replication-incompetent after one cycle of replication in the host cell.

According to the present invention the live attenuated vaccine strain that originates from modified cell lines that constitutively express HA and into such modified cell lines, 8 plasmids encoding for vRNA as well as proteins of ΔHA, NA, PB1, PB2, PA, NP, M and NS are co-tranfected so that the complementary cell lines expresses head less stem regions of Hemagglutinin (ΔHA) in the vaccine strain, keeping its structural integrity intact along with fully functional HA (hereinafter referred as fHA), another surface glycoprotein known as neuraminidase, matrix protein, nucleoprotein and all other necessary proteins present in wild strain and their respective vRNAs. Such modified cell lines can be selected from MDCK cells, Vero cells, Per.C6 cells, BHK cells, PCK cells, CHO cells, MDBK cells, HEK 293 cells (e.g., 293T cells), and COS cells. It has been already proven that headless HA or headless HA (hereinafter referred as ΔHA) is highly conserved among all influenza virus strains and evoking high titres of antibody response, the ΔHA is proven to confer high and broad range of protection against influenza strains. Therefore, it raises antibodies for having better cross protective nature or universal protection against type A and type B strains.

According to another embodiment, it has been already proven that headless HA or headless HA (ΔHA) is highly conserved among all influenza virus strains and evoking high titers of antibody response, the ΔHA is proven to confer high and broad range of protection against influenza strains. Therefore, it raises antibodies for having better cross protective nature or universal protection against type A and type B strains. The presentation invention includes an inventive strategy of incorporating this ΔHA in whole virus form of influenza and this is the key step that in our novel process of developing not only a engineered live attenuated vaccine strain but a universal vaccine strain so as to elicit broad range of immune response among hosts to confer cross protection against pan influenza strains.

The non-replicating attenuated viral strain has only ΔHA and neuraminidase as two major glycoproteins along with other Matrix protein, nucleoprotein and all other necessary proteins present in wild strain. It is designed in such a way that it can provide protection for all the high-risk groups including persons 65 years of age and older, children younger than 2 years of age, pregnant women, and people of any age who have certain medical conditions, such as chronic heart, lung, kidney, liver, blood, or metabolic diseases like diabetes, compromised immune systems, or morbid obesity from both pandemic and seasonal influenza.

The above mentioned vaccine according to the present invention, having only ΔHA, is incapable of binding the host cell receptor as it lacks globular head region. As a result, it cannot infect host cell and thus cannot replicate even for single cycle. Thus this vaccine strain does not mimic wild type in terms of infection so that the high risk group shall not develop any accidental influenza infection due to lack/poor immunity or any other adverse side effects. However, since it has the necessary immunogens namely ΔHA (for broad protection), neuraminidase, matrix protein and nucleoprotein, this completely attenuated vaccine strain has potential to evoke influenza specific broad range immune response and protection against natural and/or deliberate infection of influenza.

The present vaccine strain also consists M2 (Matrix protein) and NA (Neuraminidase) in the said attenuated viral strains with potential to provide heterosubtypic protection. Therefore, vaccine strains with HA, ΔHA, NA, M2 and other structural and nonstructural proteins intact provides a broad and cross protective efficacy. The outcome of the present innovation is to have two different vaccine strains from same production pipeline. One that possess HA in terms of functional surface exposed glycoproteins and without genetic materials of HA within the vaccine strain having one cycle replication capability within the host which is meant for non-risk groups or adults. And the same strain loses its functional HA glycoprotein after just one cycle of replication in host cell or any other cell line (For example HEK293T, MDCK, VERO, Per.C6, BHK, PCK, CHO, MDBK, HEK 293T, and COS). At this point the vaccine strain is non infective, cannot undergo replication any more. This strain can be used for high risk groups. The present invention overcomes the common challenges associated with influenza virus including genetic reassortment and reversion back to wild strain.

According to a preferred embodiment of the present invention the vaccine strain possess three different glycoproteins namely (1) functional or complete hemagglutinin (fHA or HA) (having sequence SEQ ID 2, 36 and 38 and nucleotide sequence SEQ ID 1, 35 and 37) with the variable globular head and the conserved stalk region; (2) headless hemagglutinin (ΔHA) (having sequence SEQ ID 1, 18, 20, 22, 24, 26, 28 and 30 and nucleotide sequence SEQ ID 3, 19, 21, 23, 25, 27, 29 and 31) having only conserved stalk region and (3) functional neuraminidase (NA) (having sequence SEQ ID 6, 40 and 42 and nucleotide sequence SEQ ID 5, 39 and 41). At genome level, the viral strain consists of eight negative-sense RNA segments encoding for (1) headless hemagglutinin (ΔHA), (2) neuraminidase (NA), (3) RNA polymerase subunit (PB1) (having nucleotide sequence SEQ ID 7 and 43 and protein sequence SEQ ID 8 and 44), (4) RNA polymerase subunit (PB2) (having nucleotide sequence SEQ ID 9 and 45 and protein sequence SEQ ID 10 and 46), (5) RNA polymerase subunit (PA) (having nucleotide sequence SEQ ID 11 and 47 and protein sequence SEQ ID 12 and 48), (6) nucleoprotein (NP) (having nucleotide sequence SEQ ID 13 and 49 and protein sequence SEQ ID 14 and 50), (7) two matrix proteins (M1 and M2) (having nucleotide sequence SEQ ID 15 and 51 and protein sequence SEQ ID 16 and 52) and (8) non-structural protein (NS) (having nucleotide sequence SEQ ID 17 and 53 protein sequence SEQ ID 18 and 54). The functional or complete hemagglutinin (fHA) is contributed to this viral strain by a modified mammalian cell line termed “complementary cell line”.

“Complement cell line” herein is characterized as any cell line that is genetically manipulated with the incorporation of hemagglutinin (HA) structural gene under the regulation of suitable promoter and terminator with significant expression potential so as to encode for the “functional HA protein (fHA or HA)” involved in the receptor mediated binding of the virus to the host cell and thus play a major role in its invasion.

Co-transfection of this complementary cell line with 8 plasmids encoding for vRNA as well as proteins of ΔHA (having nucleotide sequence SEQ ID 3 and protein sequence SEQ ID 4), NA (having nucleotide sequence SEQ ID 5 and protein sequence SEQ ID 6), PB1 (having nucleotide sequence SEQ ID 7 and protein sequence SEQ ID 8), PB2 (having nucleotide sequence SEQ ID 9 and protein sequence SEQ ID 10), PA (having nucleotide sequence SEQ ID 11 and protein sequence SEQ ID 12), NP (having nucleotide sequence SEQ ID 13 and protein sequence SEQ ID 14), M (having nucleotide sequence SEQ ID 15 and protein sequence SEQ ID 16) and NS (having nucleotide sequence SEQ ID 17 and protein sequence SEQ ID 18) resulted in a primary attenuated virus seed with 3D structure identical to that of the wild influenza virus with an additional antigenic structure headless HA (ΔHA), with stalk portion only, derived from vRNA plasmid. Also, HA with the stalk and globular portion, is derived through the respective protein complemented by animal cell line. This ensures a structurally intact cell-culture based vaccine. This resultant modified influenza virus strain was deposited with the ATCC under deposition number PTA-125122. The vaccine according to the present invention is rapid as the virus back bone for replication-incompetent vaccine strain is ever ready and adapted to cell lines which are a pre-requisite for response during outbreaks.

In the present invention the transmembrane domain of the influenza HA was replaced with ΔHA. The ΔHA in a live attenuated strain herein is non functional with any change of amino acid sequence or atleast one deleted or mutated nucleotide which codes for ΔHA with its same viral cassette to be expressed in the viral strain to make it non functional or inactive and still retaining its structural integrity and presenting the stem region of the HA for having broad protection.

According to yet another aspect of the present invention there is provided a vaccine comprising a virus comprising a genome engineered to express a nucleic acid encoding a polypeptide, wherein said polypeptide comprises:
a. a headless hemagglutinin comprising an N-terminal region of an HA-1 domain of a hemagglutinin covalently linked to a linker which is in turn covalently linked to a C-terminal region of the HA-1 domain of the hemagglutinin which is in turn covalently linked to an HA-2 domain of the hemagglutinin, and
b. a headless hemagglutinin comprising an N-terminal region of an HA-1 domain of a hemagglutinin covalently linked to a C-terminal region of the HA-1 domain of the hemagglutinin which is in turn covalently linked to an HA-2 domain of the hemagglutinin.

The live attenuated vaccine strain according to the present invention is expressed in an eukaryotic cell under conditions which express the HA and permits its assembly with other viral genome and release of the viral strains. The eukaryotic cell can be selected from the group consisting of a yeast cell, an insect cell, an amphibian cell, an avian cell, a plant cell or a mammalian cell.

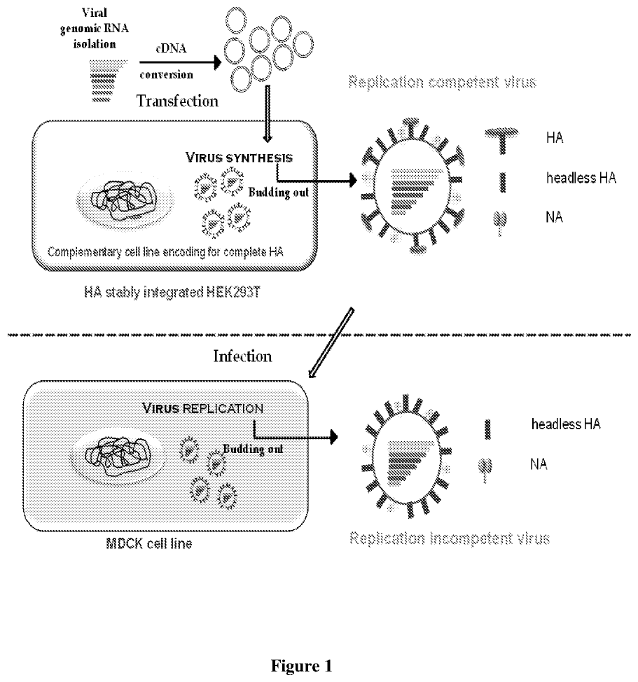

In one of the embodiment of the present invention the single cycle replication based live attenuated influenza virus can be further used to infect any unmodified eukaryotic cell to develop a live attenuated vaccine strain that expresses only ΔHA on the surface and cannot replicate in any in vitro or in vivo cells or cell lines as shown in FIG. 1. Such unmodified eukaryotic cell can be selected from the group consisting of a yeast cell, an insect cell, an amphibian cell, an avian cell, a plant cell or a mammalian cells, in vitro or in vivo cells or cell lines.

Preferably the immunogenic composition is capable of eliciting an immune response when administered to a human or non-human animal. Preferably the immune response elicited is both an antibody response and a T-cell response. Preferably all other viral proteins are also expressed inside the cells and packaged into the virus particles to optimize the cross-reactive T-cell and B-cell immune response. The immune response may be therapeutic and/or prophylactic. The immunogenic composition may be a vaccine.

The composition may further comprise an adjuvant, wherein an adjuvant enhances the protective efficacy of the composition. Suitable adjuvants will be well known to those skilled in the art, and may include emulsifiers, muramyl dipeptides, avridine, MF59, aqueous adjuvants such as aluminum hydroxide, chitosan-based adjuvants, monophosphoryl Lipid A and any of the various saponins, oils, and other substances known in the art, such as Amphigen, LPS, bacterial cell wall extracts, bacterial DNA, CpG sequences, synthetic oligonucleotides and combinations thereof. Other suitable adjuvants can be formed with an oil component, such as a single oil, a mixture of oils, a water-in-oil emulsion, or an oil-in-water emulsion. The oil may be a mineral oil, a vegetable oil, or an animal oil. Mineral oils are liquid hydrocarbons obtained from petrolatum via a distillation technique, and are also referred to in the art as liquid paraffin, liquid petrolatum, or white mineral oil. Suitable animal oils include, for example, cod liver oil, halibut oil, menhaden oil, orange oil and shark liver oil, all of which are available commercially. Suitable vegetable oils, include, for example, canola oil, almond oil, cottonseed oil, corn oil, olive oil, peanut oil, safflower oil, sesame oil, soybean oil, and the like. Freund's Complete Adjuvant (FCA) and Freund's Incomplete Adjuvant (FIA) are two common adjuvants that are commonly used in vaccine preparations, and are also suitable for use in the present invention. Both FCA and FIA are water-in-mineral oil emulsions; however, FCA also contains a killed Mycobacterium sp.

The composition may also comprise polymers or other agents to control the consistency of the composition, and/or to control the release of the protein from the composition.

The composition may also comprise other agents such as diluents, which may include water, saline, glycerol or other suitable alcohols etc; wetting or emulsifying agents; buffering agents; thickening agents for example cellulose or cellulose derivatives; preservatives; detergents; antimicrobial agents; and the like.

Preferably the active ingredients in the composition are greater than 50% pure, usually greater than 80%> pure, often greater than 90%> pure and more preferably greater than 95%, 98% or 99% pure. With active ingredients approaching 100% pure, for example about 99.5% pure or about 99.9% pure, being used most often.

The composition according to the invention may be for oral, systemic, parenteral, topical, mucosal, intramuscular, intravenous, intraperitoneal, intradermal, subcutaneous, intranasal, intravaginal, intrarectal, transdermal, sublingual, inhalation or aerosol administration. Preferably the composition is for intranasal administration.

Preferably the immune response elicited by a composition of the invention is effective against one or more influenza strains.

Preferably the immune response elicited by the composition of the invention affects the ability of the influenza virus to infect an immunized animal.

Preferably the ability of influenza to infect a human immunized with the composition of the invention is impeded or prevented. This may be achieved in a number of ways. The immune response elicited may recognize and destroy influenza virus. Alternatively, or additionally, the immune response elicited may impede or prevent the replication of the influenza virus. Alternatively, or additionally, the immune response elicited may impede or the prevent influenza virus causing disease in the human or non-human animal. Preferably the immune response elicited is directed to at least influenza A.

The composition of the invention may also comprise a further one or more antigens, in addition to the modified virus. The further antigens may also be derived from an influenza virus, and may be capable of eliciting an immune response directed to the influenza virus.

The composition may be used to elicit/produce a protective immune response when administered to a subject. The protective immune response may cause the influenza virus to be killed upon infecting the subject, or it may prevent or inhibit the influenza virus from replicating and/or from causing disease.

The composition may be used as a prophylactic or a therapeutic vaccine directed to the influenza virus, and in particular to influenza A.

The composition may be used as part of a prime boost vaccination regimen. The composition may be used in an initial prime vaccination, where the boost may be with a protein antigen. This would allow time for protein to be produced, for example in an epidemic or pandemic situation, and would also reduce costs. It is much cheaper to produce a composition of the invention than to produce a protein-based vaccine.

According to a further aspect, the invention provides a pharmaceutical composition comprising a virus or a composition of the invention and a pharmaceutically acceptable carrier or excipient.

Preferably the pharmaceutical composition comprises a modified influenza virus according to the invention.

Preferably the pharmaceutical composition is capable of producing a protective immune response to influenza.

The phrase “producing a protective immune response” as used herein means that the composition is capable of generating a protective response in a host organism, such as a human or a non-human mammal. Preferably a protective immune response protects against subsequent infection by an influenza virus. The protective immune response may eliminate or reduce the level of infection by reducing replication of the influenza virus, or by affecting the mode of action of the influenza virus, to reduce disease. Preferably the protective response is directed to at least influenza A.

Suitable acceptable excipients and carriers will be well known to those skilled in the art. These may include solid or liquid carriers. Suitable liquid carriers include water and saline. The proteins of the composition may be formulated into an emulsion or they may be formulated into biodegradable microspheres or liposomes.

The composition of the present invention may be used as a vaccine against infections caused by the influenza virus, in particular by influenza A. The vaccine may be administered prophylactically to those at risk of exposure to the influenza virus, and/or therapeutically to persons who have already been exposed to the influenza virus.

Preferably, if the composition is used as a vaccine, the composition comprises an immunologically effective amount of modified virus. An “immunologically effective amount” of a virus is an amount that when administered to an individual, either in a single dose or in a series of doses, is effective for treatment or prevention of infection by influenza. This amount will vary depending upon the health and physical condition of the individual to be treated and on the antigen. Determination of an effective amount of an immunogenic or vaccine composition for administration to an organism is well within the capabilities of those skilled in the art.

The composition may be arranged to be administered as a single dose or as part of a multiple dose schedule. Multiple doses may be administered as a primary immunization followed by one or more booster immunizations. Suitable timings between priming and boosting immunizations can be routinely determined. A composition according to the invention may be used in isolation, or it may be combined with one or more other immunogenic or vaccine compositions, and/or with one or more other therapeutic regimes.

According to a further aspect, the present invention provides use of the modified influenza virus in preparation of a medicament for eliciting an immune response. The medicament may be used for the prophylactic or therapeutic vaccination of subjects against influenza.

According to a yet further aspect, the invention provides a composition comprising the modified influenza virus for use in generating an immune response to influenza. The immune response may be prophylactic or therapeutic.

The composition may be for use as a vaccine.

According to a still further aspect, the present invention provides a method of protecting a human or non-human animal from the effects of infection by the influenza virus comprising administering to the human or non-human animal a composition according to any other aspect of the invention. The composition may be a vaccine.

According to another aspect, the invention provides a method for raising an immune response in a human or non-human animal comprising administering a pharmaceutical composition according to the invention to the human or non-human animal. The immune response is preferably protective. The method may raise a booster response in a patient that has already been primed. The immune response may be prophylactic or therapeutic.

In an embodiment, the invention provides effective therapeutic treatment comprising administration of a composition according to the invention involves monitoring for influenza virus infection after administration of the composition. One way to check the efficacy of a prophylactic treatment comprising administration of a composition according to the invention involves monitoring immune responses to influenza virus after administration of the composition.

According to another aspect, the invention provides the use of the modified influenza virus in the preparation of a medicament for use in the immunization of human or non-human mammals against infection by the influenza virus.

According to a further aspect the invention provides a kit comprising the immunogenic composition comprising the live attenuated influenza vaccine strain, and adjuvants and/or diluents and an instruction leaflet relating to administration.

Such kit is used for inducing an immune response in an organism, comprising an immunogenic or vaccine composition according to the invention and instructions leaflet relating to administration.

In addition to their use as vaccines, compositions according to the invention may be useful as diagnostic reagents and as a measure of the immune competence of a vaccine.

According to a further embodiment of the present invention there is provided a method of generating an immune response to influenza in a subject, the method comprising administering to the subject an effective amount of the immunogenic composition according to the process of release of attenuated viral strain from any cell line.

The composition can be administered mucosally, intradermally, subcutaneously, intramuscularly, or orally. The immune response vaccinates the subject against multiple strains or subtypes of influenza. The subject is preferably human, ferrets, non-human primates, birds and swine.

It will be appreciated that optional features applicable to one aspect of the invention can be used in any combination, and in any number. Moreover, they can also be used with any of the other aspects of the invention in any combination and in any number. This includes, but is not limited to, the dependent claims from any claim being used as dependent claims for any other claim in the claims of this application.

It is to be understood that the foregoing descriptive matter is illustrative of the disclosure and not a limitation. While considerable emphasis has been placed herein on the particular features of this disclosure, it will be appreciated that various modifications can be made, and that many changes can be made in the preferred embodiments without departing from the principles of the disclosure. Those skilled in the art will recognize that the embodiments herein can be practiced with modification within the spirit and scope of the embodiments as described herein. Similarly, additional embodiments and features of the present disclosure will be apparent to one of ordinary skill in art based upon description provided herein.

Descriptions of well-known/conventional methods/steps and techniques are omitted so as to not unnecessarily obscure the embodiments herein. Further, the disclosure herein provides for examples illustrating the above described embodiments, and in order to illustrate the embodiments of the present disclosure certain aspects have been employed. The examples used herein for such illustration are intended merely to facilitate an understanding of ways in which the embodiments herein may be practiced and to further enable those of skill in the art to practice the embodiments herein. Accordingly, the following examples should not be construed as limiting the scope of the embodiments herein.

The present invention is now illustrated by way of non-limiting examples.

### Example 1

### Viral Genome Fragments

The genome segments (NA, M, NP, NS, PB1, PB2 and PA along with their respective UTR and packaging sequences) of A/WSN/33 H1N1 influenza strain were synthesized. The HA genome segment of A/WSN/33 strain was modified into two formats namely ΔHA and HA were also synthesized. In case of ΔHA, the genome segment includes both UTR and packaging sequence, whereas, HA has only protein encoding sequence but not UTR and packaging sequence. Segments of ΔHA, NA, M, NP, NS, PB1, PB2 and PA are cloned into compatible vectors that encodes for both viral RNA (vRNA) and viral proteins. HA segment is cloned into a compatible vector that enables stable integration of HA in mammalian cell line (ex. HEK 293T, MDCK, Vero, Per.C6, BHK, PCK, CHO, MDBK, COS) upon transfection so that the cell line stably expresses HA protein that localizes on the cell membrane of the transfected cell line.

### Generation of Complementary Cell Line

HEK 293T cell line procured from American Type Culture Collection (ATCC) and grown in UltraCULTURE™ Serum-free Medium (Cat. No. 12-725F, Lonza) supplemented with 1× Glutamax (Cat. No. 35050061, Thermo Scientific) and 1× Penicillin-Streptomycin-Amphotericin B (Code: 17-745E, Lonza) at 37° C. in a humidified atmosphere of 5% CO₂, as recommended by the supplier. The cells are seeded in tissue culture flasks (Greiner bio-one, US) and allowed to grow to 80-90% confluency. Cells are dislodged with 0.025% (w/v) trypsin/0.01% (w/v) EDTA (Sigma, India), counted using a heamocytometer and seeded into 24 well plates (5×10⁵ cells/well). Cells are further incubated to reach 80-90% confluency at 37° C. in a humidified atmosphere of 5% CO₂. The cells are subjected to lipofectamine mediated transfection with the vector containing HA segment. The transfected cells are selected with appropriate antibiotic and the stable transfectants “complementary cell line/HA/H1N1” are expanded and maintained in presence of the antibiotic.

Generation of Live Attenuated H1N1 Influenza Particles (1^{st} Cycle Virus Seed from FIG. 1)

1×10⁶ cells of HEK 293T complementary cell line/HA/H1N1 are cultivated in T25 flask to reach 90% confluency and is subjected to PBS wash for two times. These cells are transfected with 1 microgram each of the 7 vectors-ΔHA, NA, NP, PB1, PB2, PA, M of H1N1 strain using lipofectamine. Six hours later, the DNA-transfection reagent mixture was replaced by Opti-MEM containing 0.3% BSA and 0.01% FCS. At different times after transfection, virus is harvested from the supernatant and titrated in naïve HEK 293T cells to analyze the recovered transfectant virus by plaque purification.

Generation of Replication Incompetent H1N1 Influenza Particles (2^{nd} Cycle Viral Progeny from FIG. 1)

1×10⁶ cells of naive HEK 293T cell lines are cultivated in T25 flask to reach 90% confluency and is subjected to PBS wash for two times. One ml of the live attenuated H1N1 influenza particles (1st cycle virus seed from FIG. 1) is mixed with 1 ml of virus growth medium containing UltraCULTURE™ Serum-free Medium (Cat. No. 12-725F, Lonza) supplemented with 1× Glutamax (Cat. No. 35050061, Thermo Scientific) and 1 mcg of TPCK-treated trypsin from bovine pancreas (Code: T1426, Sigma Aldrich). This virus preparation is added to the flask after the cells are washed with PBS twice. The infected monolayer is incubated at 37° C. in presence of 5% CO2 for 1 h with occasional shaking of the flask.6. After 1 h add additional 4 ml of the virus growth medium and incubate at 37° C. in presence of 5% CO₂ for 7 days or until the cytopathic effects wherein the cells display rounding up and complete detachment from flask are observed.

The “complementary cell line/HA/H1N1” and the “replication incompetent H1N1 influenza vaccine strain” have been deposited to ATCC as per Budapest Treaty under the deposit designation PTA-125121.

### Example 2

The mammalian cell line is genetically manipulated with the incorporation of haemagglutinin (HA) of H5N1 strain (i.e., H5 subtype of haemagglutinin) structural gene under the regulation of RNA polymerase I promoter and terminator so as to encode for the “functional HA protein (HA/H5)”. The resultant complementary cell line is termed “complementary cell line/HA/H5N1”.

Into this modified complementary cell line/HA/H5N1, 8 plasmids encoding for influenza vRNA and influenza proteins (wherein all the H5N1 viral genome segments except that of HA will be retained in functional form; whereas HA segment will be headless so as to code for the stalk portion of HA) is co-transfected. The resultant non-functional protein is termed as headless HA (ΔHA). These plasmids contain cDNA of all H5N1 viral genome segments except that of HA as it is complemented in complementary cell line/HA/H5N1 and thus result in structural and non-structural proteins of the virus strain. This technique results in primary attenuated virus seed termed as H5N1/HA with 3D structure identical to that of the wild H5N1 with an additional antigenic structure headless HA (ΔHA), with stalk portion with or without globular domain lacking receptor binding site, derived from vRNA plasmid. Also, HA with the stalk and globular portion, is derived through the respective protein complemented by complementary cell line/HA/H5N1.

### Example 3

### Materials and Methods

### Cells and Viruses

293T human embryonic kidney (HEK) cells and Madin-Darby canine kidney cells (MDCK) were maintained in BioWhittaker™ Dulbecco's modified Eagle medium (DMEM) (Cat. No. 12-604F, Lonza) containing with 10% fetal bovine serum, South American origin to preserve (Cat. No. 29101; MP Biomedicals, LLC) and 1× Penicillin-Streptomycin-Amphotericin B (Code: 17-745E, Lonza) in modified Eagle's medium (MEM) containing 5% fetal bovine serum and 1× Penicillin-Streptomycin-Amphotericin B, respectively. All cells were maintained at 37° C. in 5% CO₂. Influenza viruses A/PR/8/34 (H1N1), A/California/7/2009 (H1N1), A/HKx31 (H3N2) and A/Wisconsin/67/2005 (H3N2) were propagated in MDCK cells using infection medium (virus growth medium) containing MEM, BSA (0.3%), and L-(tosylamido-2-phenyl) ethyl chloromethyl ketone (TPCK) treated trypsin (1 μg/ml) and 1× Penicillin-Streptomycin-Amphotericin B at 37° C. for 40-72 h.

### Antibodies

Anti-influenza A virus HA monoclonal antibody (Cat. No. SC80550, Santa Cruz), monoclonal anti-human Influenza A (H1N1, H2N2) (Clone C179) (Cat. No. M145, Takara Bio), recombinant human Anti-IAV H5HA antibody (CR6261) (Cat. No. TAB-337CL, Creative Biolabs), anti-nucleoprotein antibody (HB-65), Influenza A nucleoprotein antibody (2F205) Alexa Fluor® 532 (Novus Biologicals).

### Example 4

### Design of Headless Haemagglutinin (ΔHA) of A/Michigan/45/2015

In the wild type influenza virus, the haemagglutinin protein typically comprises of highly variable head/globular domain (HA1) and contrastingly highly conserved stalk domain (HA2). The immune response evoked by the variable globular domain is narrowly strain-specific and thus not a promising target to develop vaccine against newly emerged or drifted strains of influenza. On the other hand, the stalk domain, in contrast, is more conserved among influenza A (group 1 and 2) viruses allowing antibodies that target this region to neutralize a wide spectrum of influenza virus subtypes. Therefore, the stalk domain is considered as a highly promising target for the development of a universal influenza vaccine, a vaccine that confers protection to any existing and/or emerging influenza strains. Therefore, it can raise antibodies for having better cross protective nature or universal protection against type A and type B strains. It has been already proven that headless HA confers high and broad range of protection against influenza strains. The HA1 domain represents signal peptide and highly variable globular domain. Within the variable HA1 globular domain a sialic acid binding site is present. This binding site is located in the distal top of haemagglutinin and it is defined as a pocket of amino acids that are highly conserved among influenza virus strains (Rohm et al., 1996). This pocket of amino acids is conserved in almost all influenza virus strains that are involved in the binding activity and effects of mutations and inhibitors on the binding affinity were examined (Al-Majhdi, 2007). Without this globular domain and/or the sialic acid binding site, the virus will not be able to bind to the sialic acid receptor on the host cell membrane and thus will be unable to establish contact with the host cell in order to initiate its replication cycle. The HA2 domain represents fusion peptide, stalk domain, transmembrane domain and cytoplasmic domain. Polypeptide representing the complete HA2 domain is highly conserved. Modified haemagglutinin in the present invention consists of the only HA1 domain with or without modified conserved HA1 globular domain so as to lack the sialic acid receptor binding site and is termed as headless haemagglutinin (ΔHA). Headless haemagglutinin is designed to incorporate on any influenza virus strain of interest as an additional glycoprotein, along with HA and NA, so that this new additional glycoprotein gives raise to broadly protective antibodies in the host, when the engineered influenza strain is administered as a vaccine. Along with the broadly protective antibodies, the immune response induced by the headless haemagglutinin also contributes broad range of protection against homologous, heterologous and heterosubtypes of influenza virus.

The HA nucleotide sequence of A/Michigan/45/2015 virus encoding for full length haemagglutinin was modified to consist the highly conserved regions of both HA1 and HA2 domain. The HA1 domain represents signal peptide (amino acids from 1 to 17) and highly variable globular domain (amino acids from 18 to 343) which is used to determine the variant of haemagglutinin in a given influenza virus. However, this HA1 globular domain has a sialic acid binding site located in the distal top of haemagglutinin and it is defined as a pocket of amino acids that are highly conserved among influenza virus strains (Rohm et al., 1996). This pocket of amino acids is conserved in almost all influenza virus strains that are involved in the binding activity and effects of mutations and inhibitors on the binding affinity will be examined (Al-Majhdi, 2007). The HA2 domain (amino acids from 345 to 566) represents fusion peptide, stalk domain, transmembrane domain (amino acids from 530 to 550) and cytoplasmic domain (amino acids from 551 to 566). Polypeptide representing the complete HA2 domain (amino acids from 345 to 566) is highly conserved. Modified haemagglutinin which consists of the conserved HA2 domain along HA1 domain devoid of the globular domain is termed as headless haemagglutinin (ΔHA). A total of three variants of ΔHA was designed and the design is described below:

4a. Headless HA Comprising of Stalk Domain and Conserved Globular Domain Portions Linked with Linker (ΔHA/H1N1-I)

This variant of HA included HA1 domain comprising of signal peptide (amino acids from 1 to 17), truncated/trimmed region of globular region devoid of receptor binding site (amino acids from 18 to 59 linked, with a G4S glycine linker, to amino acids from 292 to 343) and HA2 domain, comprising of fusion peptide, stalk domain, transmembrane domain (amino acids from 530 to 550) and cytoplasmic domain (amino acids from 551 to 566). This variant encodes for a polypeptide of HA with only signal peptide and stalk domain and no globular domain. This polypeptide is highly conserved among influenza strains and thus capable of inducing broadly reactive, protective and especially broadly protective immune response when incorporated in an influenza vaccine composition. SEQ ID Nos. 1 and 2 are representative nucleotide and protein sequence, respectively, of ΔHA/H1N1-I.

4b. Headless HA Comprising of Stalk Domain and Globular Domain Lacking Receptor Binding Site Linked with Linker (ΔHA/H1N1-11)

This variant of HA included HA1 domain comprising of signal peptide (amino acids from 1 to 17), truncated/trimmed region of globular region devoid of receptor binding site (amino acids from 18 to 107 linked, with a G4S glycine linker, to amino acids from 243 to 343) and HA2 domain, comprising of fusion peptide, stalk domain, transmembrane domain (amino acids from 530 to 550) and cytoplasmic domain (amino acids from 551 to 566). This variant encodes for a polypeptide of HA with only signal peptide and stalk domain and partial portion of globular domain that is completely lacking a pocket of amino acids representing the receptor binding site. Here, the partial portion of globular domain includes critical HA residues which are exceedingly conserved across most subtypes of influenza A viruses and represents protective determinant capable of inducing broadly neutralizing antibodies (Bangaru et al., 2019). This polypeptide is also highly conserved among influenza strains and thus capable of inducing anti-stalk and anti-head antibodies against influenza. This kind of immune response would be broadly reactive and protective when incorporated in an influenza vaccine composition. SEQ ID Nos. 3 and 4 are representative nucleotide and protein sequence, respectively, of ΔHA/H1N1-II.

4c. Headless HA Comprising of Stalk Domain and Globular Domain Lacking Receptor Binding Site Linked without Linker (ΔHA/H1N1-III)

This variant of HA included HA1 domain comprising of signal peptide (amino acids from 1 to 17), truncated/trimmed region of globular region devoid of receptor binding site (amino acids from 18 to 107 linked with amino acids from 243 to 343) and HA2 domain, comprising of fusion peptide, stalk domain, transmembrane domain (amino acids from 530 to 550) and cytoplasmic domain (amino acids from 551 to 566). SEQ ID Nos. 5 and 6 are representative nucleotide and protein sequence, respectively, of ΔHA/H1N1-III.

### Example 5

### Design of Headless Haemagglutinin (ΔHA) of A/Kansas/14/2017 (H3N2)

Headless haemagglutinin of A/Kansas/14/2017 (H3N2) consists of the conserved HA2 domain along HA1 domain devoid of the globular domain is termed as headless haemagglutinin (ΔHA). A total of three variants of ΔHA was designed and the design is described below:

5a. Headless HA Comprising of Stalk Domain and Conserved Globular Domain Portions Linked with Linker (ΔHA/H3N2-I)

This variant of HA included HA1 domain comprising of signal peptide (amino acids from 1 to 16), truncated/trimmed region of globular region devoid of receptor binding site (amino acids from 17 to 68 linked, with a G4S glycine linker, to amino acids from 294 to 344) and HA2 domain, comprising of fusion peptide, stalk domain, transmembrane domain (amino acids from 530 to 550) and cytoplasmic domain (amino acids from 551 to 566). SEQ ID Nos. 7 and 8 are representative nucleotide and protein sequence, respectively, of ΔHA/H3N2-I.

5b. Headless HA Comprising of Stalk Domain and Globular Domain Lacking Receptor Binding Site Linked with Linker (ΔHA/H3N2-II)

This variant of HA included HA1 domain comprising of signal peptide (amino acids from 1 to 16), truncated/trimmed region of globular region devoid of receptor binding site (amino acids from 17 to 113 linked, with a G4S glycine linker, to amino acids from 245 to 344) and HA2 domain, comprising of fusion peptide, stalk domain, transmembrane domain (amino acids from 530 to 550) and cytoplasmi domain (amino acids from 551 to 566). SEQ ID Nos. 9 and 10 are representative nucleotide and protein sequence, respectively, of ΔHA/H3N2-II.

5c. Headless HA Comprising of Stalk Domain and Globular Domain Lacking Receptor Binding Site Linked without Linker (ΔHA/H3N2-III)

This variant of HA included HA1 domain comprising of signal peptide (amino acids from 1 to 16), truncated/trimmed region of globular region devoid of receptor binding site (amino acids from 17 to 113 linked with amino acids from 245 to 344) and HA2 domain, comprising of fusion peptide, stalk domain, transmembrane domain (amino acids from 530 to 550) and cytoplasmic domain (amino acids from 551 to 566). SEQ ID Nos. 11 and 12 are representative nucleotide and protein sequence, respectively, of ΔHA/H3N2-III.

### Example 6

### Design of PFV

PFV is a replicative human vaccine developed against influenza. One vaccine is designed to confer broad range of protection. This novel vaccine has been developed so as mimic wild strain in conformation and undergo only certain cycles of replication upon administration to mimic infection and thus activate immune response. It harbours any one variant (ΔHA-I or ΔHA-II or ΔHA-III) of highly conserved haemagglutinin (HA) stalk region, globular head region of HA, neuraminidase (NA), matrix protein (M) and nucleoprotein (NP) to collectively elicit broadly reactive and protective antibodies by triggering both adaptive and innate immunity against flu infection. During an outbreak, using our innovative technology wherein the necessary virus backbone is readily available, the HA and NA components can be isolated/recovered from the infected patients and vaccine inoculum can be made available for administration within 14 days.

### Example 7

### Design of PFV-S

PFV-S is a non-replicative human influenza vaccine. This novel vaccine has been developed so as mimic wild strain in conformation and mimics wild virus in structure and thus induces influenza specific immune response. It harbours highly conserved haemagglutinin (HA) stalk region, neuraminidase (NA), matrix protein (M) and nucleoprotein (NP) to collectively elicit broadly reactive and protective antibodies by triggering both adaptive and innate immunity against flu infection. It is specifically designed for administration in high risk group (<6 yr and >49 yr), pregnant women and new born for safety. During an outbreak, using our innovative technology wherein the necessary virus backbone is readily available, the HA and NA components can be isolated/recovered from the infected patients and vaccine inoculum can be made available for administration within 14 days.

### Example 8

### Generation of Complementary Cell Line

Puromycin-resistant HEK 293T and MDCK cell lines stably expressing complete haemagglutinin (HA) protein from A/Michigan/45/2015 (H1N1) were established by transfection of HEK 293T and MDCK cells separately with plasmid containing the genes encoding for functional complete HA protein (HA) of A/Michigan/45/2015 flanked between RNA polymerase I promoter and RNA polymerase I terminator and puromycin resistance for stable integration of the functional HA cDNA into the genome of HEK 293T and MDCK. The stable HEK 293T clone (HEK/HA) and MDCK cell clone (MDCK/HA) expressing complete HA were selected in medium containing 2 μg/ml of puromycin (Sigma Aldrich, USA) by screening with indirect immuno-staining using an anti-influenza A virus HA monoclonal antibody (Cat. No. SC80550, Santa Cruz).

### Example 9

### Immuno-Staining Assay

HEK 293T/HA and MDCK/HA cells were plated at 1×10⁵ cells per well onto glass tissue culture chamber slides in a 12-well plate. After incubation for 72 h at 37° C. in presence of 5% CO₂ to achieve 85-90% confluency, cells were fixed with 4% paraformaldehyde for 10 min, The fixative was removed by 2 washes with PBS. Cells were incubated in PBS containing 0.2% Triton X-100 (PBST) for 5 min at room temperature and then rinsed twice with PBS. Expression of HA was detected by incubating the fixed cells with monoclonal anti-human Influenza A (H1N1, H2N2) (Clone C179) (Cat. No. M145, Takara Bio) diluted in 1% BSA in PBST as primary antibody for 1 h at room temperature. To remove excess antibody, the slides were washed 3 times in PBST. TRITC-conjugated goat anti-mouse IgG antibody (Sigma-Aldrich, USA) diluted in 1% BSA in PBST was used as secondary antibody and incubated with the cells for 1 h at room temperature. After washed 3 times, the processed cells were observed for fluorescence using a fluorescence microscope.

### Example 10

### Generation and Rescue of Replication Competent Virus Particles (PFV)

The complementary HEK/HA cell was used for generation of attenuated influenza vaccine strain. To achieve this, all the 8 individual viral segments were cloned in plasmids so as to express all the 8 vRNAs and respective proteins (ΔHA and NA from A/Michigan/45/2015, M, NP, NS, PA, PB1 and PB2 from A/PR/8/34). Headless HA here was either ΔHA/H1N1-II or ΔHA/H1N1-III. All the viral genome segments except that of HA were retained in functional form; whereas HA segment was truncated/trimmed so as to code for the stalk portion of the protein only. All the gene fragments of influenza viruses including headless HA were synthesized and cloned by GenScript Inc, USA. Resultant 8 plasmids expressing influenza vRNA was transfected into 293T/HA for virus propagation. At 48 hours post-transfection, recombinant novel virus having three major glycoproteins (HA complemented by the complementary HEK/HA cell line, NA and ΔHA) on its envelope were harvested, plaque purified for rescue and used to inoculate MDCK/HA for the production of stock viruses. This recombinant novel virus that harbors three major glycoproteins (HA complemented by the complementary HEK/HA cell line, NA and ΔHA) on its envelope is continuously replicative only in HEK/HA and MDCK/HA cells. This replication competent virus strain is termed as “Pentavalent Flu Vaccine”. PFV is first of its kind influenza virus strain on which envelope, a third glycoprotein i.e., headless haemagglutinin with only stalk region with more access to immune system, is expressed and yet the virus is made replication competent continuously in HA complementing cells and replicative for one cycle only in any other eukaryotic cells which sialic acid receptor and/or in chicken eggs, avian and mammals.

### Example 11

### Generation and Rescue of Non-Replicative Virus Particles (PFV-S)

The recombinant novel virus (PFV) that harbors three major glycoproteins (HA, NA and ΔHA) on its envelope was propagated using complementary MDCK/HA cells in infection medium to generate virion particles with both HA derived from complement cell line and ΔHA derived from vRNA plasmid. Further the cell debris is centrifuged and resultant supernatant containing those virion particles is subjected to density gradient centrifugation in order to purify the virion particles. Resultant virion particles are infected into normal 293T human embryonic kidney cells in bulk for mass production of the attenuated vaccine strains. At this step, the virion particles invade the cell lines to the complement cell line derived HA and replicates within 293T and the resultant virion particles that buds out of it will be possessing ΔHA derived from the vRNA, making it attenuated in terms of invasion i.e., non-replicative, yet structurally and antigenically similar to the wild strain. This non-replicative virus strain is termed as “PFV-S”. In this novel technology of generating two types of novel live attenuated influenza virus strains (replication-competent and non-replicative) in single production pipeline and this is first of its kind (FIG. 1).

### Example 12

### Dot Blot Assay

In order to confirm proper folding of HA and ΔHA in PFV and ΔHA in PFV-S, dot blot assay was performed using monoclonal anti-human Influenza A (H1N1, H2N2) (Clone C179) and recombinant human Anti-IAV H5HA antibody (CR6261). CR6261 is a monoclonal neutralizing antibody that binds to a broad range HA stem of the influenza virus. The epitope in the stalk region to which CR6261 binds is exceptionally conserved (Ekiert et al., 2009). Similarly, C179 is also a broadly neutralizing HA stem antibody and it binds to an epitope that is conserved on the membrane-proximal stem region of HA (Dreyfus et al., 2013). Both the antibodies inhibit the low-pH conformational change of the HA by binding to their respect epitopes and thus neutralizes the virus from establishing infection (Ekiert et al., 2009; Dreyfus et al., 2013), indicating that they strictly bind to properly folded stem region of HA. Thus these antibodies were used to confirm folding of HA and ΔHA in the mutant viruses. Wild influenza virus strain A/PR/8/34 was used as a positive control. Briefly, 1000 each of ultracentrifuged PFV, PFV-S and PR8 virus samples was loaded into the dot-blot wells, along with PBS as negative control. Samples are then filtered through a nitrocellulose membrane using vacuum on a bio-dot apparatus (Catalog No. 1706545, BioRad). The membrane is blocked in 1% filtered BSA in PBS for 1 hour at room temperature with shaking, followed by 1 h incubation with 6 μg/ml C179 or CR6261 antibody diluted in blocking buffer at 37° C. for 2 h. Horseradish peroxidase conjugated anti-mouse secondary antibody in case of C179 and anti-human secondary antibody in case of CR6261 were used for detection after developing using OPD substrate.

### Example 13

### Virus Titration

Viral titers were determined by the focus forming assay in MDCK cells. Cell culture supernatants were transferred to 96-well plates containing confluent MDCK cells. The supernatants were serially diluted ten-fold with infection medium and incubated with MDCK cells for 24 h under standard conditions. Thereafter, MDCK monolayers were subsequently washed twice with PBS and fixed with ice-cold 80% acetone for 15 min at room temperature. Viral foci were stained using a mouse monoclonal antibody against influenza A NP (Millipore, USA), goat anti-mouse IgG conjugated with biotin (Sigma-Aldrich, USA), peroxidase labeled streptavidin (MP Biomedicals, USA) and 3-amino-9-ethylcarbazole (AEC) (Sigma-Aldrich, USA). Focus-forming units (FFU) (NP-positive red-colored cells located apart from another one at a distance of two uncolored cells) were then calculated and viral titers were expressed as FFU per ml. Alternatively, viral foci were stained using Alexa Fluor® 532 conjugated Influenza A nucleoprotein antibody (2F205) (Novus Biologicals) and focus-forming units were calculated under fluorescent microscope and viral titers were expressed as FFU per ml.

### Example 14

### Plaque Assay

Plaque assays were performed in MDCK cells. Monolayers were inoculated with 100 μl of virus dilutions and allowed to adsorb for one hour at 37° C. Later, the virus inoculum was removed and cells were covered the Avicel RC-581 overlay medium and cultures were incubated at 37° C. in 5% CO₂ atmosphere. After three days of incubation, the overlay was removed by suction and the cells were fixed with 10% formalin and stained with 1% crystal violet solution in 20% methanol in water.

### Example 15

### Virus Growth Kinetics Assay

MDCK/HA and MDCK cells were infected in duplicate wells of 24-well plates with the wild-type (A/PR/8/34) or mutant virus (PFV) at a multiplicity of infection (MOI) of 0.001, overlaid with virus growth medium containing MEM, BSA (0.3%) and TPCK treated trypsin (1 μg/ml) 0.5 g of trypsin per ml, and incubated at 37° C. At select time points (0 h, 12 h, 24 h, 36 h, 48 h, and 72 h), culture supernatants were collected and stored at −80° C. until virus titration analysis. Viral titers were determined by real time PCR assay of the culture supernatants collected. RNA extraction from collected culture supernatant was done using QIAamp® Viral RNA Mini Kit by following manufacturer's instructions (Qiagen, Hilden, Germany). Briefly, an aliquot of 280 μl of the culture supernatant was inactivated in 560 μl of AVL buffer-carrier, mixed by pulse-vortexing for 15 s, and incubated for 10 min at room temperature. Then, 560 μl of absolute ethanol was added to the sample and the sample was mixed by pulse-vortexing for 15 s. The solution was applied in two parts to a QIAamp mini spin column and centrifuged at 6000 g for 1 min. The washing buffers AW1 and AW2 were added consecutively to the column and centrifuged at 6000 and 20,000 g for 1 and 3 min, respectively. The RNA was eluted with 70 μl of AVE buffer. Real time PCR was performed for the eluted RNA samples using One-Step TB Green PrimeScript RT-PCR Kit II (Takara) following manufacturer's instructions. PCR mixtures contained 100 ng of isolated RNA, 10 μl of 2× One Step TB Green RT-PCR Buffer 4, 0.8 μl each of PrimeScript 1 step Enzyme Mix 2, forward and reverse primers amplifying matrix viral gene of influenza (InfA forward: GACCRATCCTGTCACCTCTGAC and InfA reverse: AGGGCATTYTGGACAAAKCGTCTA) in a reaction volume of 20 μl. The PCR assay was performed using following conditions: reverse transcription for 30 min at 50° C., Taq inhibitor inactivation for 2 min at 95° C., and 45 cycles of denaturation for 15 s at 95° C. and annealing/extension for 30 s at 55° C. Fluorescence data (SYBR Green) was collected during the 55° C. incubation step. Remaining culture supernatant samples were subjected to focus forming assay to simultaneously determine virus titer following the method as mentioned above.

### Example 16

### Virus Spread Assay

Spread assay was conducted to analyze the capacity of PFV to undergo continuous or single-cycle replication in MDCK/HA and unmodified non-complementing MDCK cells respectively. The cells were plated at 1×10⁵ cells per well onto glass tissue culture chamber slides in a 12-well plate. Both the cells were subjected to infection with PFV in infection medium at an MOI of 0.001. After 2 h, the virus was removed, cells were washed one time in PBS, and infection medium was replenished. After incubation for 72 h at 37° C. in presence of 5% CO₂, cells were fixed with 4% paraformaldehyde for 30 min. The fixative was removed by 2 washes with PBS. Cells were incubated in PBS containing 0.5% Triton X-100 (PBST) for 5 min at room temperature and then rinsed twice with PBS. Viral antigen was detected by incubating the fixed cells with mouse monoclonal antibody against influenza A NP (Millipore, USA) diluted in 1% BSA in PBST as primary antibody for 1 h at room temperature. To remove excess antibody, the slides were washed 3 times in PBST. TRITC-conjugated goat anti-mouse IgG antibody (Sigma-Aldrich, USA) diluted in 1% BSA in PBST was used as secondary antibody and incubated with the cells for 1 h at room temperature. After washed 3 times, the processed cells were observed for fluorescence using a fluorescence microscope.

### Example 17

### Experimental Infection

Six-week-old female BALB/c mice, anesthetized with isoflurane, were infected intranasally with either 3×10⁶ or 3×10⁵ or 3×10⁴ PFU of the mutant virus (PFV and PFV-S). Wild type A/PR8/34 (1×10³ PFU) was used as a reference virus strain. Two animals from each group were euthanized on 3 days and 6 days post-infection and lungs and nasal turbinates were harvested for virus titration by NP staining method.

### Example 18

### Immunization and Protection

6-8 weeks-old female BALB/c female mice were randomly divided into 4 groups. Group 1 (n=44; sub-group n¹=22) was immunized with 3×10⁵ PFU (equivalent to 4 HAU) replication-competent PFV (PFV) once (only primed; n=22) or twice (prime on day 0 and boost on day 28; n=22) by intranasal route. Group 2 (n=44; sub-group n¹=22) was immunized with 3×10⁵ PFU non-replicative PFV-S (PFV-S) once (only primed; n=22) or twice (prime on day 0 and boost on day 28; n=22) by intranasal route. Group 3 control group received 3×10⁵ PFU heat inactivated A/PR/8/34 wild virus (n=44; sub-group n¹=22) once (only primed; n=22) or twice (prime on day 0 and boost on day 28; n=22) by intranasal. route. Group 4 mice once received sterile 1×PBS as carrier (n=22). once samples were collected from individual animals from either sub-mandibular vein or retro orbital sinus in order to collect sera in accordance with the study design schedule in Table 1. Thus collected sera samples were preserved at −20° C. and used to measure specific IgG and IgA levels against PFV by ELISA. Six weeks post-immunization schedule, 11 mice from each group were challenged with 32 HAU equivalent A/PR/8/34 (H1N1) influenza strain. Rest 11 mice from each group were challenged with 32 HAU equivalent A/HKx31 (H3N2) influenza strain. Three animals from each group were euthanized on day 3 post-infection and lungs were harvested for virus titration by NP staining method. Virus titers were expressed as the mean PFU per gram of the tissue. The body weights of the 8 mice per group were recorded on every day basis until 14 days post-infection. Body weight was monitored daily and mice losing greater than 25% of their initial weight were sacrificed and scored as dead.

TABLE 1
Mice immunization schedule

| Mice | | | | | | | Group |
|---|---|---|---|---|---|---|---|
| group | Group 1 | | Group 2 | | Group 3 | | 4 |
| (No. | PFV | PFV | PFV-S | PFV-S | Heat inactivated | | Sterile |
| of | prime | booster | prime | booster | A/PR/ | A/PR/ | PBS |
| mice) | (22) | (22) | (22) | (22) | 8/34 (22) | 8/34 (22) | (22) |
| Dose | 3 × | 3 × | 3 × | 3 × | 3 × | 3 × | 50 μl |
| (PFU) | 10⁵ | 10⁵ | 10⁵ | 10⁵ | 10⁵ | 10⁵ | |
| | on | on | on | on | on | on | |
| | day | day | day | day | day | day | |
| | 0 | 0 | 0 | 0 | 0 | 0 | |
| | — | 3 × | — | 3 × | — | 3 × | 50 μl |
| | | 10⁵ | | 10⁵ | | 10⁵ | |
| | | on | | on | | on | |
| | | day | | day | | day | |
| | | 28 | | 28 | | 28 | |

### Example 19

### Antibody Titer

Sera were collected from mice immediately prior to challenge. Virus-specific immunoglobulin G (IgG) and IgA antibody titers were measured in sera of the immunized mice by use of an enzyme-linked immunosorbent assay (ELISA). For ELISA, 96-well plates (Nunc, ThermoFisher) were coated with 0.25 μg per well of 0.5% Triton X-100 treated PFV in PBS, washed twice in PBST and blocked with 3% BSA. Sera samples (1:1000 dilution) were incubated on the virus coated plates for 1 hour at 37° C. and, after extensive washing, bound antibody was detected with a horseradish peroxidase linked rabbit anti-mouse IgG and rabbit anti-mouse IgA antibody and TMB substrate (Sigma). In each assay serum obtained from a PBS injected BALB/c mouse was included as a negative control.

### Example 20

### Haemagglutination Inhibition (HAI) Assay

A standard HAI assay was performed to assess functional antibody levels. Serum samples were treated with RDE overnight at 37° C. followed by heat inactivation for 1 h at 56° C. Two fold dilutions of RDE-treated serum samples were incubated with A/PR/8/34 (H1N1), A/California/7/2009 (H1N1), A/HKx31 (H3N2) and A/Wisconsin/67/2005 (H3N2) viruses (8 hemagglutination units per well) and 50 μl of a 0.5% suspension of turkey red blood cells for 30 min at room temperature. The reciprocal of the highest dilution of RDE treated serum that prevented hemagglutination was considered as HAI titer of the respective virus.

### Example 21

### Neuraminidase Inhibition Titer Determination Using ELLA Assay

ELLA (enzyme linked lectin assay) assay was performed as described by Couzens et al., 2014. To explain, fetuin (Sigma, St. Louis, Mo., USA) was diluted to 25 μg/ml in 0.1 M PBS and 100 μl added to each well to coat high-binding 96-well microtiter plates (Nalge Nunc, Rochester, N.Y., USA) and stored at 4° C. and used within 2 months of coating. To determine the amount of virus to use in ELLA, serial dilutions of A/PR/8/34 (H1N1), A/California/7/2009 (H1N1), A/HKx31 (H3N2) and A/Wisconsin/67/2005 (H3N2) viruses were prepared in Dulbecco's PBS (pH 7.4)-0.9 mM CaCl₂-0.5 mM MgCl₂ containing 1% bovine serum albumin (BSA) and 0.5% Tween and then dispensed (50 μl/well) into fetuin-coated plates containing an equal volume of PBS. The plates were incubated for 16-18 h at 37° C., then washed 6 times with PBS-0.05% Tween 20 (PBST) before adding 100 μl peanut agglutinin (PNA) conjugated to horse-radish peroxidase (HRPO, Sigma). PNA-HRPO was used at the highest dilution that gave the maximum signal when titrated on fully digested fetuin. Plates were incubated at room temperature for 2 h and washed 3 times with PBST before adding o-phenylenediamine dihydrochloride (OPD, Sigma) to the plate. The color reaction was stopped after 10 min by the addition of 1 N H₂SO₄. The plates were read at 490 nm for 0.1 s using a 96-well plate reader (Tecan, Männedorf, Switzerland). The dilution of virus that resulted in 90-95% maximum signal was elected for use in serology. To measure the NI titers, each serum sample was heat treated (56° C. for 45 min) and then diluted serially in PBS-BSA. Fifty microliters of each dilution was added to duplicate wells of a fetuin-coated plate. An equal volume (50 μl) of the selected virus dilution was added to all serum-containing wells in addition to at least 4 wells containing diluent without serum that served as a positive (virus only) control. At least 4 wells were retained as a background control (PBS only). The plates were incubated for 16-18 h at 37° C. As described for the virus titration, the plates were washed and PNA-HRPO was added to all wells. After a 2 h incubation period, the plates were washed and peroxidase substrate (OPD) was added. The color reaction was stopped after 10 min and absorbance read. The mean absorbance of the background (A_{bkg}) was subtracted from the test wells and positive control (Aₚₒₛ) wells. The percent NA activity was calculated by dividing the mean absorbance of duplicate test wells (Aₜₑₛₜ) by the mean absorbance of virus only wells and multiplied by 100, i.e. (Aₜₑₛₜ−A_{bkg})/(Aₚₒₛ−A_{bkg})×100. To determine percent NA inhibition, the percent activity was subtracted from 100. The NI titers were defined as the reciprocal of the last dilution that resulted in at least 50% inhibition.

### Example 22

### Cytokine Analysis

Induced serum cytokine levels were quantified using Bio-plex Pro mouse Cytokine 8-plex assay kit (Catalog No. M6000003J7, Bio-Rad, California). The cytokine targets of detection included IL-1β, IL-2, IL-4, IL-5, IL-10, IFN-γ, GM-CSF and TNF-α. Multiplex assay was performed in accordance with the manufacturer's instructions using Luminex 200 system and Bioplex HTF (Bio-Rad, California). Standards and each sample were analyzed in biological triplicates. Approximately, each serum sample was diluted at 1:4 ratio using sample diluent provided by the manufacturer. Data analysis was performed using Bio-Plex Manager, version 5.0 (Bio-Rad, California). The concentration of each cytokine in samples analyzed was determined using the standard curve generated for standards provided with the kit. Serum samples from control mice were also analyzed as mentioned above. Data values were calculated by linear regression using the standard curve as a reference, as per manufacturer's instruction. Sample that showed out of range (00R) concentration was assigned to the highest extrapolated value for a conservative estimate and statistical analysis. The experiment was repeated in biological duplicates with the blood samples collected from immunization groups on 30-day post-immunization.

### Results

### Generation of PFV

Three variants of PFV were designed based on the variations in the removal of a length of globular domain. The first construct included HA1 domain comprising of signal peptide, truncated/trimmed region of globular region devoid of receptor binding site (amino acids from 18 to 59 linked, with a G4S glycine linker, to amino acids from 292 to 343) and HA2 domain, comprising of fusion peptide, stalk domain, transmembrane domain and cytoplasmic domain. This variant was made to enable increased access of the stalk domain to immune response so that broadly reactive antibodies are generated. Second construct included HA1 domain comprising of signal peptide, truncated/trimmed region of globular region devoid of receptor binding site (amino acids from 18 to 107 linked, with a G4S glycine linker, to amino acids from 243 to 343) and HA2 domain, comprising of fusion peptide, stalk domain, transmembrane domain and cytoplasmic domain. In this construct the globular region retained was highly conserved, proven to represent epitopes that have potential to induce broadly reactive and protective anti-HA antibodies and lacks receptor binding region. This construct was designed so that the globular regions resulting after trimming the receptor binding region were linked with glycine linker for proper protein folding and structural integrity of the headless HA. Third construct of headless HA was designed similar to the second variant but not to include glycine linker in order to understand the necessity of this linker to enable proper folding of the protein. All the three headless HA variants were confirmed to be properly folded as the virus with these variants showed strong reactivity with anti-human Influenza A (H1N1, H2N2) (Clone C179) and recombinant human Anti-IAV H5HA antibody (CR6261). Both C179 and CR6261 are antibodies which bind only to properly folded haemagglutinin.

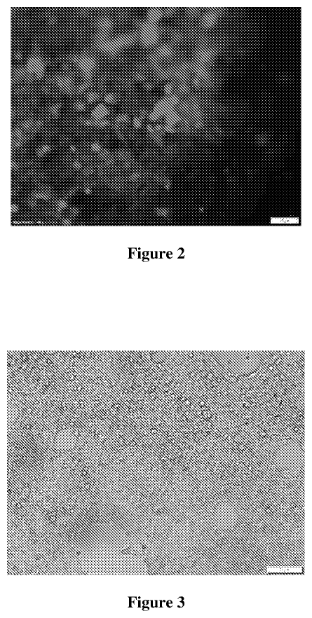

In order to generate and rescue the influenza virus strain with headless haemagglutinin as additional glycoprotein on its surface, HEK 293T and MDCK cell lines stably expressing complete functional or complete haemagglutinin (HA) protein from A/Michigan/45/2015 (H1N1) were generated. FIG. 2 illustrates the expression of functional or complete haemagglutinin on the surface of HEK 293T in which gene encoding for functional or complete haemagglutinin was stably integrated, when stained with monoclonal anti-human Influenza A (H1N1, H2N2) (Clone C179). All the three PFV variants showed successful infection in MDCK/HA and MDCK cells as confirmed by the expression of the NP which is significantly similar as the wild type A/PR/8/34 virus. To this modified HEK/HA cells, 8 individual viral segments were cloned in plasmids so as to express all the 8 vRNAs and respective proteins (ΔHA and NA from A/Michigan/45/2015, M, NP, NS, PA, PB1 and PB2 from A/PR/8/34) were co-transfected and the resultant virus was rescued at 48 hours post-transfection by plaque purification. This recombinant novel virus that harbors three major glycoproteins (HA, NA and ΔHA) on its envelope is continuously replicative only in HEK/HA and MDCK/HA cells. This replication competent virus strain is termed as “PFV”. FIG. 3 is the microscopic observation of HEK 293T/HA cells infected with replication-competent influenza virus for cytopathic effect.

### Example 23

### Generation of PFV-S

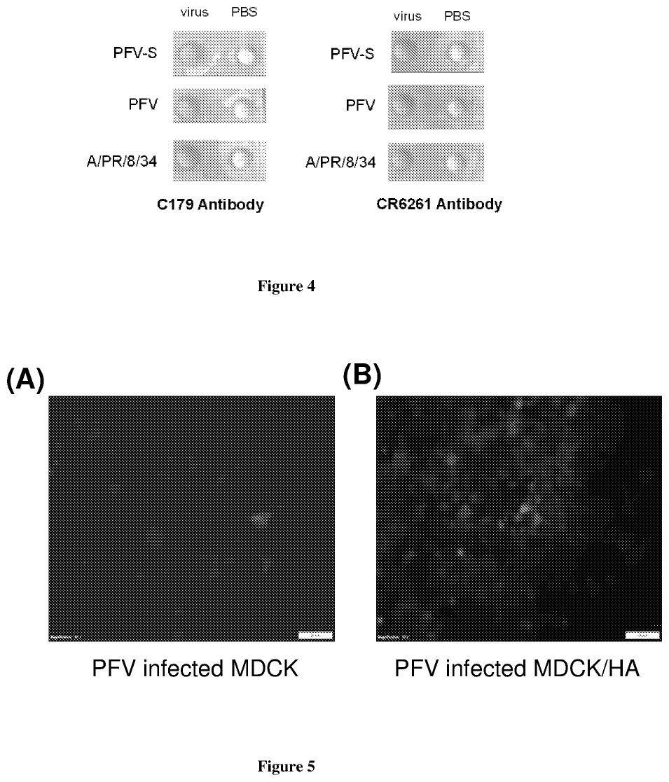

In order to generate and rescue the replication incompetent influenza virus strain (PFV-S), MDCK cells were infected with PFV at an MOI of 0.001 and 72 hr later resultant virus progeny was rescued. This recombinant novel virus that harbors only two glycoproteins (NA and ΔHA) on its envelope can no more replicate in any cells. FIG. 4 represents the dot blot assay of both PFV and PFV-S variants of influenza virus strains showing reactivity with C179 and CR6261 antibodies indicating the properly folding of HA and ΔHA on their envelope.

### Example 24

### Growth Kinetics of PFV and PFV-S In Vitro

To evaluate the replication of the rescued replication-competent virus (PFV), its spread in HA-complementing MDCK/HA and non-complementing MDCK cells was analyzed. Both cell lines were inoculated at an MOI of 0.001 and 72 hr later immunostained for the presence of virus nucleoprotein (FIG. 5). When PFV was grown on HA-complementing MDCK/HA cells, the formation of typical influenza foci was detected. However, when it was infected to non-complementing MDCK cells, only single infected cells were observed. These results demonstrated that PFV can infect cells, but the infection is restricted to the initially infected cell.

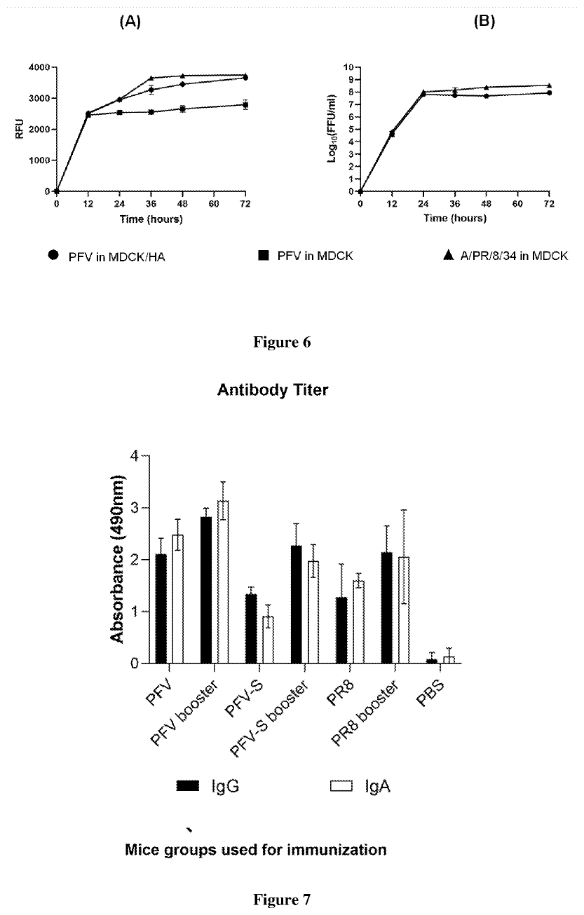

To further characterize the growth kinetics of PFV and PFV-S, multistep growth kinetics assay was conducted in which MDCK/HA or MDCK cells were infected at an MOI of 0.001 with PFV, and the presence of infectious virus was determined at various time points by real time PCR assay using InfA primer that detects matrix protein encoding gene in all strains of type A influenza virus (FIG. 6A) and also by conducting focus forming assay (FIG. 6B). Wild strain A/PR/8/34 was also used as a positive control by infecting it to MDCK cells at an MOI of 0.001. The virus titers were of similar pattern in both the experiments and thus double confirming the growth parameters of PFV. On the other hand, since PFV-S is replication-incompetent its replication-kinetics was not measured by focus-forming assay.

In this experiment, PFV infecting MDCK/HA cells showed comparatively significant and similar growth pattern as of A/PR/8/34 strain. This is because the virus progeny of PFV resulting from replication in MDCK/HA cell line had HA, ΔHA and NA making it replication-competent. Constitutive complementation of complete HA to this virus progeny enabled repeated cycles of replication similar to an unmodified wild virus strain. On the other hand, PFV infecting MDCK cells showed increase in virus titer comparable with PFV and PR8 only until 24 hours of the experiment. As the MDCK cells had no complementation of complete HA resultant virus progeny of PFV had only ΔHA and NA making it replication-incompetent and thus no further increase in titer was observed throughout the experiment duration.

### Example 25

### PFV and PFV-S are Attenuated

Attenuation of viruses in animals is essential for live vaccines. Therefore, the pathogenicity of PFV and PFV-S was compared with wild type A/PR/8/34 in BALB/c mice. Four weeks old female Balb/C mice were intranasally infected with different doses of PFV, PFV-S and the virus titers in lungs (Table 2) and body weights were determined. The wild type A/PR/8/34 strain was infected to mice for reference. When mice were infected with 3×10⁶ or 3×10⁵ PFU of PFV, virus was recovered from the lungs on 3 dpi, but titers were significantly lower when compared with that of PR8 infected mice lungs. In case of PFV-S infected mice, no virus titer was detected on both 3 and 6 dpi and no body weight loss was also recorded. No virus titer was recorded from nasal turbinate samples of both PFV and PFV-S infected mice. This indicated that both PFV and PFV-S are highly attenuated and does not cause any clinical symptoms or pathogenic effects on animals.

| | | Virus titer (mean ± standard deviation PFU/g) | | | |
|---|---|---|---|---|---|
| | | Lungs | | Nasal turbinate | |
| | PFU of virus | 3 days | 6 days | 3 days | 6 days |
| | inoculated | post- | post- | post- | post- |
| Virus | per mouse | infection | infection | infection | infection |
| PFV | 3 × 10⁴ | 3.7 ± 0.3 | 3.4 ± 0.1 | ND | ND |
| | 3 × 10⁵ | 3.9 ± 0.1 | 3.6 ± 0.2 | ND | ND |
| | 3 × 10⁶ | 4.5 ± 0.1 | 3.7 ± 0.1 | ND | ND |
| PFV-S | 3 × 10⁴ | ND | ND | ND | ND |
| | 3 × 10⁵ | ND | ND | ND | ND |
| | 3 × 10⁶ | ND | ND | ND | ND |
| PR8 | 1 × 10³ | 6.1 ± 0.2 | 6.2 ± 0.1 | 8.4 ± 0.2 | 7.8 ± 0.1 |

### Example 26

### Antibody Response to PFV and PFV-S

In order to evaluate the immunogenicity and influenza specific immune response of both PFV and PFV-S, mice were immunized with 3×10⁵ PFU of PFV or PFV-S on a vaccination regimen of either one dose or two does separated by 28 days. Mice immunized with 3×10⁵ PFU heat inactivated PR8 on a regimen of either one dose or two does separated by 28 days were referred as positive control group and group of mice which received sterile PBS were considered as negative control. To examine PFV-specific antibody production, IgG and IgA titers in serum were detected in all groups of mice (FIG. 7). Both the virus-specific IgG and IgA levels in serum from mice immunized twice with PFV were higher than the respective titers from all other groups of mice of the vaccination regiment. Notably, in case of both the mice groups which received PFV once and twice respectively, IgA levels were higher than IgG levels. Serum samples from mice groups which received PFV-S also had significantly high levels of both IgG and IgA titers. However, when compared to PR8 immunized groups, PFV-S immunized groups had higher titers of IgG, but lesser titers of IgA. The higher titers of virus-specific IgG among PFV-S immunized mice indicated that the replication-incompetent virus which had only headless haemagglutinin on its surface had induced anti-stalk IgG antibodies which could be considered by HAI assay. No virus-specific IgG and IgA titer was recorded in case of PBS group of mice. These results demonstrate that both PFV (prime and booster immunized) and PFV-S (booster immunized) could elicit influenza virus-specific humoral immunity more efficiently than heat inactivated PR8.

### Example 27

### Assessment of HA Head and Stalk Antibodies Among PFV and PFV-S Immunized Mice by Determining Anti-Influenza Hemagglutination Inhibition (HAI) Titer

Immunogenicity studies in mice were conducted to evaluate the immunogenic potential of replication-competent PFV and replication-incompetent PFV-S influenza virus strains and to determine the minimal effective dose that generates protective correlates of immunity. Two sets of immunization experiment were performed using prime/boost strategy. In the first immunization, experiment groups of mice were vaccinated intranasally with 3×10⁵ PFU of the mutant virus and boosted once at 28 days intervals. The control group received only PBS. Group of mice immunized with 3×10⁵ PFU of heat inactivated A/PR/8/34 virus and boosted once at 28 days intervals served as positive control group. Following immunization, the mice showed no adverse effects suggesting that both PFV and PFV-S vaccine was well tolerated. The results of the ELISA test demonstrated that they are strongly immunogenic. We quantified serum titers of HA head-specific Abs against A/PR/8/34 (H1N1), A/California/7/2009 (H1N1), A/HKx31 (H3N2) and A/Wisconsin/67/2005 (H3N2) strains using hemagglutination inhibition (HAI) assays. HAI assays detect HA head-specific Abs that prevent influenza virus-mediated cross-linking of red blood cells. For this assay, the pooled sera from four mice were tested to pinpoint the differences in protective efficacy of each of the vaccine doses. The level of HAI antibodies was tested after the final vaccine doses and HAI titer of >40 was considered as protective antibody response against influenza infections. Accordingly, HAI titers of PFV, PFV-S and heat inactivated PR8 immunized groups (both prime and booster) showed protective antibody response. Groups of mice immunized with PFV showed HAI titers as high as 1280 and significant differences among groups (Table 3). To explain, a single administration of the PFV elicited specific anti-HA antibodies with an endpoint titer of 1280 against A/PR/8/34 (H1N1), 640 against A/California/7/2009 (H1N1), 640 against A/HKx31 (H3N2) and 320 against A/Wisconsin/67/2005 (H3N2) viruses. Two doses administration of PFV elicited specific anti-HA antibodies with an endpoint titer of 1280, 640, 640 and 320 against A/PR/8/34 (H1N1), A/California/7/2009 (H1N1), A/HKx31 (H3N2) and A/Wisconsin/67/2005 (H3N2) viruses respectively. HAI antibody titers in the group immunized once with single dose and booster dose PFV-S was 160 and 320 against/PR/8/34 (H1N1), 160 against A/California/7/2009 (H1N1), 80 and 160 against A/HKx31 (H3N2), and 40 and 80 against A/Wisconsin/67/2005 (H3N2) viruses tested indicated that this mutant virus had induced high and broadly reactive anti-HA stalk antibodies with anti-haemagglutination property. Clearly, PFV (single/booster) immunization induced higher and broadly reactive HAI titer in comparison with PFV-S as well as the positive control i.e., heat inactivated PR8.

TABLE 3
Anti-influenza Hemagglutination Inhibition (HAI) titer

| | | Anti- | Anti- | Anti- | Anti- |
|---|---|---|---|---|---|
| | No. | PR8 | X31 | A/California/ | A/Wisconsin/ |
| Vaccine/ | of | HAI | HAI | 7/2009 H1N1 | 67/2005 H3N2 |
| Immunogen | doses | titer | titer | HAI titer | HAI titer |
| PFV | 2 | 1280 | 640 | 640 | 320 |
| PFV | 1 | 1280 | 320 | 640 | 320 |
| PFV-S | 2 | 320 | 160 | 160 | 80 |
| PFV-S | 1 | 160 | 80 | 160 | 40 |
| PR8 | 2 | 640 | 640 | 320 | 320 |
| PR8 | 1 | 320 | 160 | 160 | 80 |
| PBS | 2 | <10 | <10 | <10 | <10 |

### Example 28

Protection by vaccination with NA largely depends on the induction of antibodies that can mediate neuraminidase inhibition (NI). Therefore, to examine the potential breadth of the antibody response directed against NA component of both PFV and PFV-S, heat-inactivated sera raised against these mutant viruses in immunized mice were compared with sera from mice that had been immunized with wild-type A/PR/8/34 for their capacity to mediate NI against A/PR/8/34 (H1N1), A/California/7/2009 (H1N1), A/HKx31 (H3N2) and A/Wisconsin/67/2005 (H3N2) viruses. An IC₅₀ titer (1:x dilution resulting in 50% NA inhibition) above the lowest dilution of sera tested was considered as a positive response. PFV anti-sera mediated NI against A/Wisconsin/67/2005 and A/Hk31, a lesser extent (on average close to half of the cut-off point) against A/PR/8/34 ana A/California/7/2009 viruses (Table 4). PFV-S anti-sera exceptionally and strongly inhibited NA activity of all four virulent viruses tested. Finally, anti-PR8 NA serum showed considerable NI against all viruses tested but was below the cut-off point. This could be due to the disruption of epitopes in the NA protein due to the heat inactivation of virus.

TABLE 4
Anti-sera raised to PFV and PFV-S mediates NI against a broader
range of influenza A viruses than wild type NA sera.

| | No. | NI titer showing 50% inhibition | | | |
|---|---|---|---|---|---|
| Vaccine/ | of | A/PR/ | A/ | A/California/ | A/Wisconsin/ |
| Immunogen | doses | 8/34 | HKx31 | 7/2009 | 67/2005 |
| PFV | 2 | 10 | 10 | 10 | 10 |
| PFV | 1 | 10 | 20 | 10 | 10 |
| PFV-S | 2 | 10 | 20 | 20 | 20 |
| PFV-S | 1 | 20 | 10 | 10 | 10 |
| PR8 | 2 | 20 | 80 | 80 | 10 |
| PR8 | 1 | 10 | 40 | 40 | ND* |
| PBS | 2 | <10 | <10 | <10 | <10 |

ND*—not detected

### Example 29

### PFV and PFV-S Elicits Influenza-Specific T Cell Responses

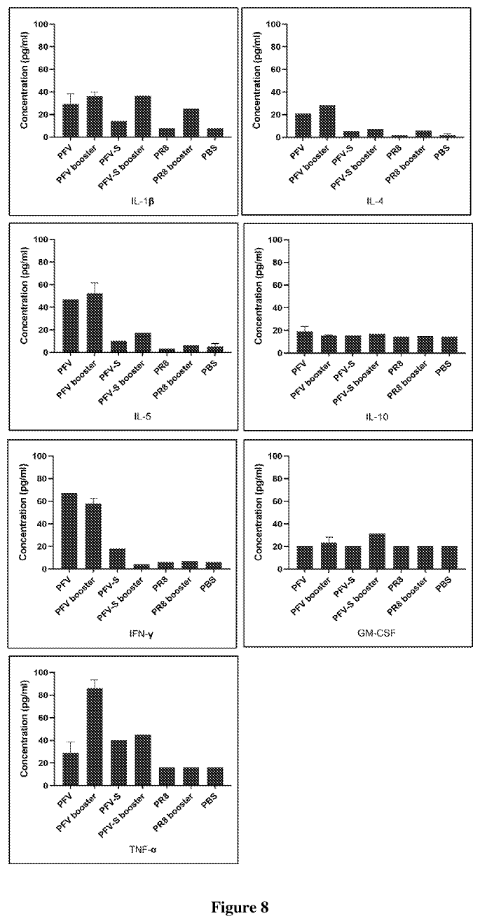

Lymphocyte cytokine profiles in BALB/c mice after immunization with PFV and PFV-S (prime/booster) were analyzed by ELISA using PBMCs isolated from PFV, PFV-S and PR8 immunized mice blood samples in order to determine T cell response. Cytokine levels for IL-1β, IL-2, IL-4, IL-5, IL-10, IFN-γ, GM-CSF and TNF-α secreted by lymphocytes from each immunization group are shown in FIG. 8. Briefly, cytokine concentrations induced by PFV in mice included moderate levels of pro-inflammatory cytokines, IFN-γ and TNF-α indicating induction of Type 1 T helper (Th1) polarized immune response along with moderate levels of IL-1β along with anti-inflammatory cytokines IL-4 and IL-5 indicating Type 2 T helper (Th2) immunity also. Virtually, no IL-2, IL-10, and GM-CSF was recorded. Similar cytokine profile was observed in mice those received booster dose of PFV. In case of PFV-S immunized mice, cytokine concentrations were low and moderate levels TNF-α indicating induction of Th1 based immune response and IL-1β indicating Th2 immunity also. Very less concentrations of IL-4, IL-5, IFN-γ and GM-CSF were measured and IL-2 and IL-10 were below detection limits. Similar cytokine profile was observed in mice those received booster dose of PFV-S. In case of PR8 immunized mice (both prime and booster dose groups) induced moderate levels of IL-1β. Other cytokines including IL-4, IL-5, IL-10, IFN-γ, GM-CSF and TNF-α were of very less concentration. No IL-2 was recorded. IL-10, IFN-γ, GM-CSF and TNF-α among prime and booster immunization groups of mice. By contrast, lymphocytes of PBS injected mice were unable to secrete the aforementioned cytokines. The production of the pro-inflammatory mediators i.e., IFN-γ and TNF-α reflected the Type 1 T helper (Th1) polarized response that would result in increased macrophage activation and subsequent host resistance to intracellular infection. Both cytokines are involved in the formation of well-differentiated, macrophages that play an essential role in preventing the proliferation and dissemination of intracellular pathogens. On the other hand, IL-4 and IL-5 secretion indicated induction of Type 2 Th (Th2) polarized response (humoral immunity) in mice against PFV and PFV-S. This is not consistent with the extremely high levels of cytokines that are observed during severe or lethal influenza infection and have been postulated to play a role in lung damage. Taken together these data suggest that PFV and PFV-S induces effective protection and strong cellular immunity without inducing damaging inflammatory responses.

### Example 30

### Post Challenge Immune Responses of Vaccinated Mice

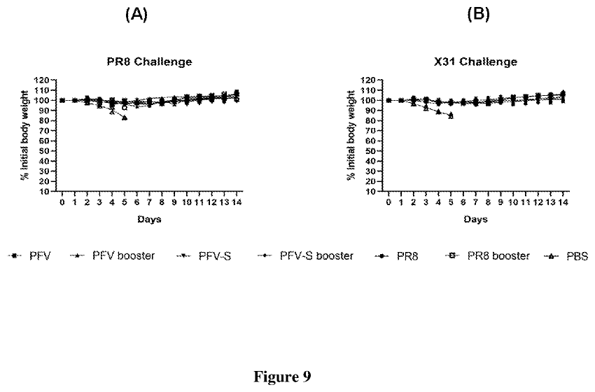

Mice immunized with the PFV and PFV-S vaccine (prime and booster) were challenged 6 weeks after immunization with 32 HAU of the wild-type A/PR8/34 (H1N1 virus) and A/HKx31 (H3N2 virus). Unlike control mice, all vaccine immunized mice survived a lethal challenge with either of the pathogenic A/PR8/34 viruses and did not show any symptoms, including weight loss, after the challenge. By contrast, all of the control mice died or had to be euthanized due to their symptoms within 14 days post challenge (FIG. 9). Taking the results together, it is concluded that the PFV and PFV-S vaccine can confer protective immunity to mice against lethal challenge with pathogenic heterosubtype A/PR8/34 as well as heterologous A/HKx31 indicated that both replication-competent and replication-incompetent mutant viruses of the present invention confers broad range of protection against influenza infection and thus qualifies as a universal influenza vaccine.

The data presented herein shows that both replication-competent and replication-incompetent influenza virus variants of the present invention are attenuated and not pathogenic as they did not induce weight loss or clinical symptoms upon administration in Balb/C mice. Also, the virus variants were not detected or detected at lower titers in mice lungs in contrary to the wild type PR8 influenza strain. The study also showed that doses of 3×10⁵ PFU of replication-competent H1N1 given once or twice intranasally to Balb/C mice induced complete protection against matching and non-matching heterosubtypic and heterologous strains of type A influenza and no weight loss or viral replication after challenge with 32 HAU of matching and non-matching heterosubtypic (A/PR/8/34 and A/California/7/2009 H1N1) and heterologous (A/Hkx31 and A/Wisconsin/67/2005 H3N2) virulent strains. Similar protection pattern was observed in case of Balb/C mice that received 3×10⁵ PFU of replication-incompetent H1N1 given once or twice intranasally. This broad range of protection could be attributed to the increased access of stalk HA domain as well as presence of other two universal vaccine candidate targets M and NP in both the vaccine variants.

After completion of immunization schedule, PFV and PFV-S vaccinated mice had a strong virus-specific IgG and IgA titers in sera. The higher titers of virus-specific IgG among PFV immunized mice indicated that the replication-competent virus which had both headless haemagglutinin and complete HA on its surface had induced both anti-head and anti-stalk IgG antibodies. The higher titers of virus-specific IgG among PFV-S immunized mice indicated that the replication-incompetent virus which had only headless haemagglutinin on its surface had induced anti-stalk IgG antibodies. These virus-specific antibodies demonstrated moderate to higher range of haemagglutination inhibition titers against heterosubtypic as well as heterologous virulent strains of H1N1 and H3N2 influenza. Both PFV and PFV-S vaccinated mice showed superior HAI titers when compared to the positive control group of mice which received heat inactivated PR8 wild type influenza virus.

The protective immune response induced in mice to PFV and PFV-S was not only broad range and strong humoral response but strong T cell response also, as witnessed by examining the cytokine profiles among vaccinated mice groups. Both PFV and PFV-S successfully induced Th1 and Th2 mediating cytokines and there was no or very limited inflammatory cytokine IL-10.

Compared with PFV-S, PFV induced higher immune response and higher titers of antibody, HAI titers, NI titers and cytokines. This could be due to the antigen amplification of PFV within the mice due to one cycle of replication. This also explains the similar cytokine profile pattern in case of PFV and PFV-S vaccinated mice, because due to the antigen amplification of PFV in mice, replication-competent PFV virus gets converted into replication-incompetent PFV-S.

This collective and broad spectrum humoral and cell mediated immunity induced by PFV and PFV-S was sufficient to embark strong protective response following challenge of immunized mice with matching and non-matching heterosubtypic (A/PR/8/34 and A/California/7/2009 H1N1) and heterologous (A/Hkx31 and A/Wisconsin/67/2005 H3N2) virulent strains. In summary, the data in the present invention shows that a replication-competent influenza virus with highly conserved headless haemagglutinin can induce broad range of heterosubtypic and heterologous immunity and protects mice from challenge with highly pathogenic strains of influenza. Also, does a replication-incompetent influenza virus. The data supports our proposal of using both replication-competent influenza virus and replication-incompetent influenza virus as broadly protective universal influenza vaccine. For safety purpose, the replicative and controllably infective variant of virus can be administered to adult group of hosts and the non-replicative yet structurally mimicking variant of virus can be administered to high risk group of hosts as a promising universal influenza vaccine.

During an outbreak, using present invention, wherein the necessary virus backbone is readily available, the HA and NA components can be isolated/recovered from the infected patients and both replication-competent and replication-incompetent vaccine inoculum can be made available for administration within 14 days.

1.-55. (canceled)

56. A modified influenza virus comprising haemagglutinin and a headless heamagglutinin, wherein the virus is a single-cycle replication competent virus, upon infection of a human or non-human animal.

57. The modified influenza virus of claim 56, wherein the haemagglutinin is selected from H1 to H18 influenza A sub-types of haemagglutinin and the headless haemagglutinin is a headless form of H1 to H18 influenza A sub-types of haemagglutinin.

58. The modified influenza virus of claim 57, wherein the haemagglutinin comprises a sequence set forth in SEQ ID Nos. 3, 19, 21, 23, 25, 27, 29, 31, and 33.

59. The modified influenza virus of claim 56, wherein the headless haemagglutinin lacks a receptor binding region of haemagglutinin and wherein the receptor binding region of haemagglutinin comprises a sequence from amino acids 58 to 295 of haemagglutinin.

60. The modified influenza virus of claim 56, wherein the headless haemagglutinin is selected from the group consisting of:
a. a headless haemagglutinin comprising an N-terminal region of an HA-1 domain of a haemagglutinin covalently linked to a linker which is in turn covalently linked to a C-terminal region of the HA-1 domain of the haemagglutinin which is in turn covalently linked to an HA-2 domain of the haemagglutinin, and
b. a headless haemagglutinin comprising an N-terminal region of an HA-1 domain of a haemagglutinin covalently linked to a C-terminal region of the HA-1 domain of the haemagglutinin which is in turn covalently linked to an HA-2 domain of the haemagglutinin.

61. The modified influenza virus of claim 60, wherein the N-terminal region of the HA-1 domain comprises amino acids 1-59, 1-68, 1-107, or 1-113 of the haemagglutinin.

62. The modified influenza virus of claim 60, wherein the C-terminal region of the HA-1 domain comprises amino acids 292-343, 243-343, 294-344, or 245-344 of the haemagglutinin.

63. The modified influenza virus of claim 56, wherein the virus comprises a viral ribonucleic acid (vRNA) encoding the headless haemagglutinin, wherein the vRNA comprises a sequence selected from the group consisting of SEQ ID Nos.: 3, 19, 21, 23, 25, 27, 29, 31, and 33.

64. The modified influenza virus of claim 63, wherein the vRNA comprises an untranslated region (UTR) at the 5′ and/or 3′ of the sequence selected from the group consisting of SEQ ID Nos.: 3, 19, 21, 23, 25, 27, 29, 31, and 33.

65. The modified influenza virus of claim 56, wherein the haemagglutinin is selected from H1 to H18 influenza A sub-types of haemagglutinin and the headless haemagglutinin comprises a sequence selected from the group consisting of SEQ ID Nos.: 4, 20, 22, 24, 26, 28, 30, 32, and 34.

66. A modified influenza virus comprising a headless haemagglutinin, wherein the virus is a replication incompetent virus.

67. The modified influenza virus of claim 66, wherein the headless haemagglutinin lacks a receptor binding region of haemagglutinin, wherein the receptor binding region of haemagglutinin comprises a sequence from amino acids 58 to 295 of the haemagglutinin.

68. The modified influenza virus of claim 66, wherein the headless haemagglutinin is selected from the group consisting of:
a. a headless haemagglutinin comprising an N-terminal region of an HA-1 domain of a haemagglutinin covalently linked to a linker which is in turn covalently linked to a C-terminal region of the HA-1 domain of the haemagglutinin which is in turn covalently linked to an HA-2 domain of the haemagglutinin, and
b. a headless haemagglutinin comprising an N-terminal region of an HA-1 domain of a haemagglutinin covalently linked to a C-terminal region of the HA-1 domain of the haemagglutinin which is in turn covalently linked to an HA-2 domain of the haemagglutinin.

69. The modified influenza virus of claim 68, wherein the N-terminal region of the HA-1 domain comprises amino acids 1-59, 1-68, 1-107, or 1-113 of the haemagglutinin.

70. The modified influenza virus of claim 68, wherein the C-terminal region of the HA-1 domain comprises amino acids 292-343, 243-343, 294-344, or 245-344 of the haemagglutinin.

71. The modified influenza virus of claim 66, wherein the virus comprises a viral ribonucleic acid (vRNA) encoding the headless haemagglutinin, wherein the vRNA comprises a sequence selected from the group consisting of SEQ ID Nos.: 3, 19, 21, 23, 25, 27, 29, 31, and 33.

72. The modified influenza virus of claim 71, wherein the vRNA comprises an untranslated region (UTR) at the 5′ and/or 3′ of the sequence selected from the group consisting of SEQ ID Nos.: 3, 19, 21, 23, 25, 27, 29, 31, and 33.

73. A vaccine composition comprising the modified influenza virus of claim 56.

74. A method of producing the modified virus of claim 56, comprising: transfecting a viral RNA encoding the headless haemagglutinin into a cell line stably expressing the haemagglutinin and harvesting the modified virus.

75. A method of producing the modified virus of claim 66, comprising: transfecting a viral RNA encoding the headless haemagglutinin into a cell line stably expressing the haemagglutinin; harvesting the modified virus comprising the haemagglutinin and the headless haemagglutinin from cell supernatant; infecting a mammalian cell line with the modified virus comprising the haemagglutinin and the headless haemagglutinin; and harvesting the modified virus comprising the headless haemagglutinin from cell supernatant.

76. The method of claim 74 or 75, wherein the cell line stably expressing the haemagglutinin is MDCK or HEK293T.

77. A method of treatment or prevention of infection by influenza comprising administering immunologically effective amount of the modified influenza virus of claim 56 to the human or non-human animal.

78. An immunogenic composition comprising the modified influenza virus of claim 56.

79. The immunogenic composition of claim 78, further comprising an adjuvant selected from emulsifiers, muramyl dipeptides, avridine, MF59, aqueous adjuvants such as aluminum hydroxide, chitosan-based adjuvants, monophosphoryl Lipid A, saponins, oils, Amphigen, LPS, bacterial cell wall extracts, bacterial DNA, CpG sequences, synthetic oligonucleotides, oil, mixture of oils, water-in-oil emulsion, oil-in-water emulsion, mineral oil, a vegetable oil, or an animal oil such as cod liver oil, halibut oil, menhaden oil, orange oil and shark liver oil, vegetable oils such as canola oil, almond oil, cottonseed oil, corn oil, olive oil, peanut oil, safflower oil, sesame oil, soybean oil, and the like. Freund's Complete Adjuvant (FCA) and Freund's Incomplete Adjuvant (FIA) and combinations thereof.

80. The immunogenic composition of claim 78, wherein said immunogenic composition comprises diluents selected from the group consisting of water, saline, glycerol or other suitable alcohols etc; wetting or emulsifying agents; buffering agents; thickening agents for example cellulose or cellulose derivatives; preservatives; detergents; antimicrobial agents; and the like.

81. The immunogenic composition of claim 78, comprising at least one single cycle based live attenuated influenza strain.

82. The immunogenic composition of claim 78, wherein said composition is adapted for oral, systemic, parenteral, topical, mucosal, intramuscular, intravenous, intraperitoneal, intradermal, subcutaneous, intranasal, intravaginal, intrarectal, transdermal, sublingual, inhalation or aerosol administration.

83. The immunogenic composition of claim 82, wherein said composition is for intranasal administration.

84. The immunogenic composition of claim 78, wherein said composition is effective against one or more influenza strains.

85. The immunogenic composition of claim 78, wherein said composition further comprises one or more antigens.

86. A method of generating genetically modified cell line in vitro comprising transforming the cell line with modified strain of claim 56, such that the attenuated influenza strain replicates constitutively.

87. The method of claim 86, wherein said modified mammalian cell line is selected from MDCK cells, Vero cells, Per.C6 cells, BHK cells, PCK cells, CHO cells, MDBK cells, HEK 293 cells, 293T cells, and COS cells.

88. The modified influenza virus strain of claim 56 encoding at least three glycoproteins comprising:
a. functional or complete haemagglutinin (fHA or HA) comprising a sequence set forth in SEQ ID NO: 2 or 35 or 37 with the variable globular head and the conserved stalk region;
b. truncated or headless haemagglutinin (tHA or ΔHA) comprising a sequence set forth in SEQ ID NO: 3 or 4 or 19 through 34 having only conserved stalk region; and
c. functional neuraminidase (NA) comprising a sequence set forth in SEQ ID NO: 5 or 6 or 39 through 42.

89. A polynucleotide sequence having headless haemagglutinin (tHA or ΔHA) with only conserved stalk region with or without globular domain lacking receptor binding site of SEQ ID NO: 3 or 4 or 19 through 34.

90. The polynucleotide sequence of claim 89 wherein the polynucleotide sequence encodes a polypeptide comprising the amino acid sequence of SEQ ID NO: 4, 18, 20, 22, 24, 26, 28, 30, 32 or 34.


SEQUENCE LISTING (54 sequences)

SEQ ID NO: 1 - DNA, 1695 bp, Artificial sequence
  Functional Heamagglutinin fHA of Influenza H1N1
atgaaggcaaaactactggtcctgttatatgcatttgtagctacagatgcagacacaatatgtataggctaccatgcgaacaactcaaccgacactgttgacacaatattcgagaagaatgtggcagtgacacattctgttaacctgctcgaagacagacacaacgggaaactatgtaaattaaaaggaatagccccactacaattggggaaatgtaacatcaccggatggctcttgggaaatccagaatgcgactcactgcttccagcgagatcatggtcctacattgtagaaacaccaaactctgagaatggagcatgttatccaggagatttcatcgactatgaggaactgagggagcaattgagctcagtatcatcattagaaagattcgaaatatttcccaaggaaagttcatggcccaaccacacattcaacggagtaacagtatcatgctcccataggggaaaaagcagtttttacagaaatttgctatggctgacgaagaagggggattcatacccaaagctgaccaattcctatgtgaacaataaagggaaagaagtccttgtactatggggtgttcatcacccgtccagcagtgatgagcaacagagtctctatagtaatggaaatgcttatgtctctgtagcgtcttcaaattataacaggagattcaccccggaaatagctgcaaggcccaaagtaaaagatcaacatgggaggatgaactattactggaccttgctagaacccggagacacaataatatttgaggcaactggtaatctaatagcaccatggtatgctttcgcactgagtagagggtttgagtccggcatcatcacctcaaacgcgtcaatgcatgagtgtaacacgaagtgtcaaacaccccagggatctataaacagcaatctccctttccagaatatacacccagtcacaataggagagtgcccaaaatatgtcaggagtaccaaattgaggatggttacaggactaagaaacatcccatccattcaatacagaggtctatttggagccattgctggttttattgaggggggatggactggaatgatagatggatggtatggttatcatcatcagaatgaacagggatcaggctatgcagcggatcaaaaaagcacacagaatgccattaacgggattacaaacaaggtgaactctgttatcgagaaaatgaacactcaattcacagctgtgggtaaagaattcaacaacttagaaaaaaggatggaaaatttaaataaaaaagttgatgatgggtttctggacatttggacatataatgcagaattgttagttctactggaaaatgaaagaactttggatttccatgacttaaatgtgaagaatctgtacgagaaagtaaaaagccaattaaagaataatgccaaagaaatcggaaatgggtgttttgagttctaccacaagtgtgacaatgaatgcatggaaagtgtaagaaatgggacttatgattatccaaaatattcagaagaatcaaagttgaacagggaaaagatagatggagtgaaattggaatcaatgggggtgtatcagattctggcgatctactcaactgtcgccagttcactggtgcttttggtctccctgggggcaatcagtttctggatgtgttctaatgggtctttgcagtgcagaatatgcatc

SEQ ID NO: 2 - protein, 565 aa, Artificial sequence
  Functional Heamagglutinin fHA, of Influenza H1N1
MKAKLLVLLYAFVATDADTICIGYHANNSTDTVDTIFEKNVAVTHSVNLLEDRHNGKLCKLKGIAPLQLGKCNITGWLLGNPECDSLLPARSWSYIVETPNSENGACYPGDFIDYEELREQLSSVSSLERFEIFPKESSWPNHTFNGVTVSCSHRGKSSFYRNLLWLTKKGDSYPKLTNSYVNNKGKEVLVLWGVHHPSSSDEQQSLYSNGNAYVSVASSNYNRRFTPEIAARPKVKDQHGRMNYYWTLLEPGDTIIFEATGNLIAPWYAFALSRGFESGIITSNASMHECNTKCQTPQGSINSNLPFQNIHPVTIGECPKYVRSTKLRMVTGLRNIPSIQYRGLFGAIAGFIEGGWTGMIDGWYGYHHQNEQGSGYAADQKSTQNAINGITNKVNSVIEKMNTQFTAVGKEFNNLEKRMENLNKKVDDGFLDIWTYNAELLVLLENERTLDFHDLNVKNLYEKVKSQLKNNAKEIGNGCFEFYHKCDNECMESVRNGTYDYPKYSEESKLNREKIDGVKLESMGVYQILAIYSTVASSLVLLVSLGAISFWMCSNGSLQCRICI

SEQ ID NO: 3 - DNA, 1014 bp, Artificial sequence
  Truncated Heamagglutinin tHA of Influenza H1N1
atgaaggcaaaactactggtcctgttatatgcatttgtagctacagatgcagacacaatatgtataggctaccatgcgaacaactcaaccgacactgttgacacaatattcgagaagaatgtggcagtgacacattctgttaacctgctcgaagacagacacaacgggaaactatgtggcggagggggctgtaacacgaagtgtcaaacaccccagggatctataaacagcaatctccctttccagaatatacacccagtcacaataggagagtgcccaaaatatgtcaggagtaccaaattgaggatggttacaggactaagaaacatcccatccattcaatacagaggtctatttggagccattgctggttttattgaggggggatggactggaatgatagatggatggtatggttatcatcatcagaatgaacagggatcaggctatgcagcggatcaaaaaagcacacagaatgccattaacgggattacaaacaaggtgaactctgttatcgagaaaatgaacactcaattcacagctgtgggtaaagaattcaacaacttagaaaaaaggatggaaaatttaaataaaaaagttgatgatgggtttctggacatttggacatataatgcagaattgttagttctactggaaaatgaaagaactttggatttccatgacttaaatgtgaagaatctgtacgagaaagtaaaaagccaattaaagaataatgccaaagaaatcggaaatgggtgttttgagttctaccacaagtgtgacaatgaatgcatggaaagtgtaagaaatgggacttatgattatccaaaatattcagaagaatcaaagttgaacagggaaaagatagatggagtgaaattggaatcaatgggggtgtatcagattctggcgatctactcaactgtcgccagttcactggtgcttttggtctccctgggggcaatcagtttctggatgtgttctaatgggtctttgcagtgcagaatatgcatc

SEQ ID NO: 4 - protein, 338 aa, Artificial sequence
  Truncated Heamagglutinin tHA of Influenza H1N1
MKAKLLVLLYAFVATDADTICIGYHANNSTDTVDTIFEKNVAVTHSVNLLEDRHNGKLCGGGGCNTKCQTPQGSINSNLPFQNIHPVTIGECPKYVRSTKLRMVTGLRNIPSIQYRGLFGAIAGFIEGGWTGMIDGWYGYHHQNEQGSGYAADQKSTQNAINGITNKVNSVIEKMNTQFTAVGKEFNNLEKRMENLNKKVDDGFLDIWTYNAELLVLLENERTLDFHDLNVKNLYEKVKSQLKNNAKEIGNGCFEFYHKCDNECMESVRNGTYDYPKYSEESKLNREKIDGVKLESMGVYQILAIYSTVASSLVLLVSLGAISFWMCSNGSLQCRICI

SEQ ID NO: 5 - DNA, 1410 bp, Artificial sequence
  Neuraminidase NA of Influenza H1N1
agcaaaagcaggagtttaaaatgaatccaaaccagaaaataataaccattgggtcaatctgtatggtagtcggaataattagcctaatattgcaaataggaaatataatctcaatatggattagccattcaattcaaaccggaaatcaaaaccatactggaatatgcaaccaaggcagcattacctataaagttgttgctgggcaggactcaacttcagtgatattaaccggcaattcatctctttgtcccatccgtgggtgggctatacacagcaaagacaatggcataagaattggttccaaaggagacgtttttgtcataagagagccttttatttcatgttctcacttggaatgcaggaccttttttctgactcaaggcgccttactgaatgacaagcattcaagggggacctttaaggacagaagcccttatagggccttaatgagctgccctgtcggtgaagctccgtccccgtacaattcaaggtttgaatcggttgcttggtcagcaagtgcatgtcatgatggagtgggctggctaacaatcggaatttctggtccagatgatggagcagtggctgtattaaaatacaaccgcataataactgaaaccataaaaagttggaggaagaatatattgagaacacaagagtctgaatgtacctgtgtaaatggttcatgttttaccataatgaccgatggcccaagtgatgggctggcctcgtacaaaattttcaagatcgagaaggggaaggttactaaatcaatagagttgaatgcacctaattctcactacgaggaatgttcctgttaccctgataccggcaaagtgatgtgtgtgtgcagagacaattggcacggttcgaaccgaccatgggtgtccttcgaccaaaacctagattataaaataggatacatctgcagtggggttttcggtgacaacccgcgtcccaaagatggaacaggcagctgtggcccagtgtctgctgatggagcaaacggagtaaagggattttcatataagtatggcaatggtgtttggataggaaggactaaaagtgacagttccagacatgggtttgagatgatttgggatcctaatggatggacagagactgatagtaggttctctatgagacaagatgttgtggcaataactaatcggtcagggtacagcggaagtttcgttcaacatcctgagctaacagggctagactgtatgaggccttgcttctgggttgaattaatcagggggctacctgaggaggacgcaatctggactagtgggagcatcatttctttttgtggtgtgaatagtgatactgtagattggtcttggccagacggtgctgagttgccgttcaccattgacaagtagtctgttcaaaaaactccttgtttctact

SEQ ID NO: 6 - protein, 453 aa, Artificial sequence
  Neuraminidase NA-Influenza H1N1
MNPNQKIITIGSICMVVGIISLILQIGNIISIWISHSIQTGNQNHTGICNQGSITYKVVAGQDSTSVILTGNSSLCPIRGWAIHSKDNGIRIGSKGDVFVIREPFISCSHLECRTFFLTQGALLNDKHSRGTFKDRSPYRALMSCPVGEAPSPYNSRFESVAWSASACHDGVGWLTIGISGPDDGAVAVLKYNRIITETIKSWRKNILRTQESECTCVNGSCFTIMTDGPSDGLASYKIFKIEKGKVTKSIELNAPNSHYEECSCYPDTGKVMCVCRDNWHGSNRPWVSFDQNLDYKIGYICSGVFGDNPRPKDGTGSCGPVSADGANGVKGFSYKYGNGVWIGRTKSDSSRHGFEMIWDPNGWTETDSRFSMRQDVVAITNRSGYSGSFVQHPELTGLDCMRPCFWVELIRGLPEEDAIWTSGSIISFCGVNSDTVDWSWPDGAELPFTIDK

SEQ ID NO: 7 - DNA, 2341 bp, Artificial sequence
  Polymerase PB1 of Influenza H1N1
agcgaaagcaggcaaaccatttgaatggatgtcaatccgactttacttttcttaaaagtgccagcacaaaatgctataagcacaactttcccttatactggagaccctccttacagccatgggacaggaacaggatacaccatggatactgtcaacaggacacatcagtactcagaaaggggaagatggacaacaaacaccgaaactggagcaccgcaactcaacccgattgatgggccactgccagaagacaatgaaccaagtggttatgcccaaacagattgtgtattggaagcaatggccttccttgaggaatcccatcctggtatctttgagacctcgtgtcttgaaacgatggaggttgttcagcaaacacgagtggacaagctgacacaaggccgacagacctatgactggactctaaataggaaccagcctgctgcaacagcattggccaacacaatagaagtgttcagatcaaatggcctcacggccaatgaatctggaaggctcatagacttccttaaggatgtaatggagtcaatgaacaaagaagaaatggagatcacaactcattttcagagaaagagacgagtgagagacaatatgactaagaaaatggtgacacagagaacaataggtaaaaggaagcagagattgaacaaaaggagttatctaattagggcattaaccctgaacacaatgaccaaagatgctgagagagggaagctaaaacggagagcaattgcaaccccagggatgcaaataagggggtttgtatactttgttgagacactagcaaggagtatatgtgagaaacttgaacaatcaggattgccagttggaggcaatgagaagaaagcaaagttggcaaatgttgtaaggaagatgatgaccaattctcaggacactgaaatttctttcaccatcactggagataacaccaaatggaacgaaaatcagaaccctcggatgtttttggccatgatcacatatataaccagaaatcagcccgaatggttcagaaatgttctaagtattgctccaataatgttctcaaacaaaatggcgagactgggaaaggggtacatgtttgagagcaagagtatgaaaattagaactcaaatacctgcagaaatgctagcaagcatcgatttgaaatacttcaatgattcaactagaaagaagattgaaaaaatccggccgctcttaatagatgggactgcatcattgagccctggaatgatgatgggcatgttcaatatgttaagtactgtattaggcgtctccatcctgaatcttggacaaaagagacacaccaagactacttactggtgggatggtcttcaatcttctgatgattttgctctgattgtgaatgcacccaatcatgaagggattcaagccggagtcaacaggttttatcgaacctgtaagctacttggaattaatatgagcaagaaaaagtcttacataaacagaacaggtacatttgaattcacaagttttttctatcgttatgggtttgttgccaatttcagcatggagcttcccagctttggggtgtctgggatcaacgagtctgcggacatgagtattggagttactgtcatcaaaaacaatatgataaacaatgatcttggtccagcaaccgctcaaatggcccttcagctgttcatcaaagattacaggtacacgtaccggtgccatagaggtgacacacaaatacaaacccgaagatcatttgaaataaagaaactgtgggagcaaacccattccaaagctggactgctggtctccgacggaggcccaaatttatacaacattagaaatctccacattcctgaagtctgcttgaaatgggaattaatggatgaggattaccaggggcgtttatgcaacccactgaacccatttgtcaaccataaagacattgaatcagtgaacaatgcagtgataatgccagcacatggtccagccaaaaacatggagtatgatgctgttgcaacaacacactcctggatccccaaaagaaatcgatccatcttgaatacaagccaaagaggaatacttgaagatgaacaaatgtaccaaaagtgctgcaacttatttgaaaaattcttccccagcagttcatacagaagaccagtcgggatatccagtatggtggaggctatggtttccagagcccgaattgatgcacgaattgatttcgaatctggaaggataaagaaagaggagttcactgagatcatgaagatctgttccaccattgaagagctcagacggcaaaaatagtgaatttagcttgtccttcatgaaaaaatgccttgtttctact

SEQ ID NO: 8 - protein, 757 aa, Artificial sequence
  Polymerase PB1 of Influenza H1N1
MDVNPTLLFLKVPAQNAISTTFPYTGDPPYSHGTGTGYTMDTVNRTHQYSERGRWTTNTETGAPQLNPIDGPLPEDNEPSGYAQTDCVLEAMAFLEESHPGIFETSCLETMEVVQQTRVDKLTQGRQTYDWTLNRNQPAATALANTIEVFRSNGLTANESGRLIDFLKDVMESMNKEEMEITTHFQRKRRVRDNMTKKMVTQRTIGKRKQRLNKRSYLIRALTLNTMTKDAERGKLKRRAIATPGMQIRGFVYFVETLARSICEKLEQSGLPVGGNEKKAKLANVVRKMMTNSQDTEISFTITGDNTKWNENQNPRMFLAMITYITRNQPEWFRNVLSIAPIMFSNKMARLGKGYMFESKSMKIRTQIPAEMLASIDLKYFNDSTRKKIEKIRPLLIDGTASLSPGMMMGMFNMLSTVLGVSILNLGQKRHTKTTYWWDGLQSSDDFALIVNAPNHEGIQAGVNRFYRTCKLLGINMSKKKSYINRTGTFEFTSFFYRYGFVANFSMELPSFGVSGINESADMSIGVTVIKNNMINNDLGPATAQMALQLFIKDYRYTYRCHRGDTQIQTRRSFEIKKLWEQTHSKAGLLVSDGGPNLYNIRNLHIPEVCLKWELMDEDYQGRLCNPLNPFVNHKDIESVNNAVIMPAHGPAKNMEYDAVATTHSWIPKRNRSILNTSQRGILEDEQMYQKCCNLFEKFFPSSSYRRPVGISSMVEAMVSRARIDARIDFESGRIKKEEFTEIMKICSTIEELRRQK

SEQ ID NO: 9 - DNA, 2341 bp, Artificial sequence
  Polymerase PB2-Influenza H1N1
agcgaaagcaggtcaattatattcaatatggaaagaataaaagaactaaggaatctaatgtcgcagtctcgcactcgcgagatactcacaaaaaccaccgtggaccatatggccataatcaagaagtacacatcaggaagacaggagaagaacccagcacttaggatgaaatggatgatggcaatgaaatatccaattacagcagacaagaggataacggaaatgattcctgagagaaatgagcagggacaaactttatggagtaaaatgaatgacgccggatcagaccgagtgatggtatcacctctggctgtgacatggtggaataggaatggaccagtgacaagtacagttcattatccaaaaatctacaaaacttattttgaaaaagtcgaaaggttaaaacatggaacctttggccctgtccattttagaaaccaagtcaaaatacgtcgaagagttgacataaatcctggtcatgcagatctcagtgccaaagaggcacaggatgtaatcatggaagttgttttccctaacgaagtgggagccaggatactaacatcggaatcgcaactaacgacaaccaaagagaagaaagaagaactccagggttgcaaaatttctcctctgatggtggcatacatgttggagagagaactggtccgcaaaacgagattcctcccagtggctggtggaacaagcagtgtgtacattgaagtgttgcatttgacccaaggaacatgctgggaacagatgtacactccaggaggggaggcgaggaatgatgatgttgatcaaagcttaattattgctgctagaaacatagtaagaagagccacagtatcagcagatccactagcatctttattggagatgtgccacagcacgcagattggtggaataaggatggtaaacatccttaggcagaacccaacagaagagcaagccgtggatatttgcaaggctgcaatgggactgagaattagctcatccttcagttttggtggattcacatttaagagaacaagcggatcatcagtcaagagagaggaagaggtgcttacgggcaatcttcagacattgaagataagagtgcatgagggatatgaagagttcacaatggttgggagaagagcaacagctatactcagaaaagcaaccaggagattgattcagctgatagtgagtgggagagacgaacagtcgattgccgaagcaataattgtggccatggtattttcacaagaggattgtatgataaaagcagttagaggtgacctgaatttcgtcaatagggcgaatcagcgattgaatcccatgcaccaacttttgagacattttcagaaggatgcaaaggtgctctttcaaaattggggaattgaatccatcgacaatgtgatgggaatgatcgggatattgcccgacatgactccaagcaccgagatgtcaatgagaggagtgagaatcagcaaaatgggggtagatgagtattccagcgcggagaagatagtggtgagcattgaccgttttttgagagttagggaccaacgtgggaatgtactactgtctcccgaggagatcagtgaaacacagggaacagagaaactgacaataacttactcatcgtcaatgatgtgggagattaatggtcctgaatcagtgttggtcaatacctatcagtggatcatcagaaactgggaaactgttaaaattcagtggtcccagaatcctacaatgctgtacaataaaatggaatttgagccatttcagtctttagttccaaaggccgttagaggccaatacagtgggtttgtgagaactctgttccaacaaatgagggatgtgcttgggacatttgataccgctcagataataaaacttcttcccttcgcagccgctccaccaaagcaaagtagaacgcagttctcctcattgactataaatgtgaggggatcaggaatgagaatacttgtaaggggcaattctccagtattcaactacaacaagaccactaaaagactcacagttctcggaaaggatgctggccctttaactgaagacccagatgaaggcacagctggagttgagtccgcagttctgagaggattcctcattctgggcaaagaagacaggagatatggaccagcattaagcataaatgaactgagcaaccttgcgaaaggagagaaggctaatgtgctaattgggcaaggagacgtggtgttggtaatgaaacggaaacggaactctagcatacttactgacagccagacagcgaccaaaagaattcggatggccatcaattagtgtcgaatagtttaaaaacgaccttgtttctact

SEQ ID NO: 10 - protein, 759 aa, Artificial sequence
  Polymerase PB2-Influenza H1N1
MERIKELRNLMSQSRTREILTKTTVDHMAIIKKYTSGRQEKNPALRMKWMMAMKYPITADKRITEMIPERNEQGQTLWSKMNDAGSDRVMVSPLAVTWWNRNGPVTSTVHYPKIYKTYFEKVERLKHGTFGPVHFRNQVKIRRRVDINPGHADLSAKEAQDVIMEVVFPNEVGARILTSESQLTTTKEKKEELQGCKISPLMVAYMLERELVRKTRFLPVAGGTSSVYIEVLHLTQGTCWEQMYTPGGEARNDDVDQSLIIAARNIVRRATVSADPLASLLEMCHSTQIGGIRMVNILRQNPTEEQAVDICKAAMGLRISSSFSFGGFTFKRTSGSSVKREEEVLTGNLQTLKIRVHEGYEEFTMVGRRATAILRKATRRLIQLIVSGRDEQSIAEAIIVAMVFSQEDCMIKAVRGDLNFVNRANQRLNPMHQLLRHFQKDAKVLFQNWGIESIDNVMGMIGILPDMTPSTEMSMRGVRISKMGVDEYSSAEKIVVSIDRFLRVRDQRGNVLLSPEEISETQGTEKLTITYSSSMMWEINGPESVLVNTYQWIIRNWETVKIQWSQNPTMLYNKMEFEPFQSLVPKAVRGQYSGFVRTLFQQMRDVLGTFDTAQIIKLLPFAAAPPKQSRTQFSSLTINVRGSGMRILVRGNSPVFNYNKTTKRLTVLGKDAGPLTEDPDEGTAGVESAVLRGFLILGKEDRRYGPALSINELSNLAKGEKANVLIGQGDVVLVMKRKRNSSILTDSQTATKRIRMAIN

SEQ ID NO: 11 - DNA, 2233 bp, Artificial sequence
  Polymerase PA of Influenza H1N1
agcgaaagcaggtactgattcaaaatggaagattttgtgcgacaatgcttcaatccgatgattgtcgagcttgcggaaaaggcaatgaaagagtatggagaggacctgaaaatcgaaacaaacaaatttgcagcaatatgcactcacttggaagtgtgcttcatgtattcagattttcacttcatcgatgagcaaggcgagtcaatagtcgtagaacttggcgatccaaatgcacttttgaagcacagatttgaaataatcgagggaagagatcgcacaatagcctggacagtaataaacagtatttgcaacactacaggggctgagaaaccaaagtttctaccagatttgtatgattacaagaagaatagattcatcgaaattggagtaacaaggagagaagttcacatatactatctggaaaaggccaataaaattaaatctgagaagacacacatccacattttctcattcactggggaggaaatggccacaaaggccgactacactctcgatgaagaaagcagggctaggatcaaaaccaggctattcaccataagacaagaaatggctagcagaggcctctgggattcctttcgtcagtccgagagaggcgaagagacaattgaagaaagatttgaaatcacaggaacaatgcgcaagcttgccgaccaaagtctcccgccaaacttctccagccttgaaaaatttagagcctatgtggatggattcgaaccgaacggctacattgagggcaagctttctcaaatgtccaaagaagtaaatgctagaattgaaccttttttgaaatcaacaccacgaccacttagacttccggatgggcctccctgttctcagcggtccaaattcctgctgatggatgccttaaaattaagcattgaggacccaagtcatgagggagaggggataccgctatatgatgcaatcaaatgcatgagaacattctttggatggaaggaacccaatgttgttaaaccacacgaaaagggaataaatccaaattatcttctgtcatggaagcaagtactggcagaactgcaggacattgagaatgaggagaaaattccaaggactaaaaatatgaagaaaacgagtcagttaaagtgggcacttggtgagaacatggcaccagaaaaggtagactttgacgattgtaaagatgtaggcgatttgaagcaatatgatagtgatgaaccagaattgaggtcgcttgcaagttggattcagaatgagttcaacaaggcatgtgaactgaccgattcaagctggatagagctcgatgagattggagaagatgcggctccaattgaacacattgcaagcatgagaaggaattatttcacagcagaggtgtctcattgcagagccacagaatacataatgaagggggtgtacatcaatactgccttgcttaatgcatcctgtgcagcaatggatgatttccaattaattccaatgataagcaagtgtagaactaaggagggaaggcgaaagaccaatttgtacggtttcatcataaaaggaagatcccacttaaggaatgacaccgatgtggtaaactttgtgagcatggagttttccctcactgacccaagacttgaaccacacaaatgggagaagtactgtgttcttgaggtaggagatatgcttctaagaagtgccataggccatgtgtcaaggcctatgttcttgtatgtgaggacaaatggaacctcaaaaattaaaatgaaatgggggatggaaatgaggcgttgcctccttcagtcacttcaacaaatcgagagtatgattgaagctgagtcctctgtcaaggagaaagacatgaccaaagagttctttgaaaacaaatcagaaacatggcccgttggagagtcccccaaaggagtggaggaaggttccattgggaaggtctgcagaactttattggcaaagtcggtattcaacagcttgtatgcatctccacaactagaaggattttcagctgaatcaagaaaactgcttcttatcgttcaggctcttagggacaacctggaacctgggacctttgatcttggggggctatatgaagcaattgaggagtgcctgattaatgatccctgggttttgcttaatgcttcttggttcaactccttcctcacacatgcattgagatagttgtggcaatgctactatttgctatccatactgtccaaaaaagtaccttgtttctact

SEQ ID NO: 12 - protein, 696 aa, Artificial sequence
  Polymerase PA of Influenza H1N1
MKEYGEDLKIETNKFAAICTHLEVCFMYSDFHFIDEQGESIVVELGDPNALLKHRFEIIEGRDRTIAWTVINSICNTTGAEKPKFLPDLYDYKKNRFIEIGVTRREVHIYYLEKANKIKSEKTHIHIFSFTGEEMATKADYTLDEESRARIKTRLFTIRQEMASRGLWDSFRQSERGEETIEERFEITGTMRKLADQSLPPNFSSLEKFRAYVDGFEPNGYIEGKLSQMSKEVNARIEPFLKSTPRPLRLPDGPPCSQRSKFLLMDALKLSIEDPSHEGEGIPLYDAIKCMRTFFGWKEPNVVKPHEKGINPNYLLSWKQVLAELQDIENEEKIPRTKNMKKTSQLKWALGENMAPEKVDFDDCKDVGDLKQYDSDEPELRSLASWIQNEFNKACELTDSSWIELDEIGEDAAPIEHIASMRRNYFTAEVSHCRATEYIMKGVYINTALLNASCAAMDDFQLIPMISKCRTKEGRRKTNLYGFIIKGRSHLRNDTDVVNFVSMEFSLTDPRLEPHKWEKYCVLEVGDMLLRSAIGHVSRPMFLYVRTNGTSKIKMKWGMEMRRCLLQSLQQIESMIEAESSVKEKDMTKEFFENKSETWPVGESPKGVEEGSIGKVCRTLLAKSVFNSLYASPQLEGFSAESRKLLLIVQALRDNLEPGTFDLGGLYEAIEECLINDPWVLLNASWFNSFLTHALR

SEQ ID NO: 13 - DNA, 1565 bp, Artificial sequence
  Nucleoprotein NP of Influenza H1N1
agcaaaagcagggtagataatcactcacagagtgacatcgaaatcatggcgaccaaaggcaccaaacgatcttacgaacagatggagactgatggagaacgccagaatgccactgaaatcagagcatctgtcggaaaaatgattgatggaattggacgattctacatccaaatgtgcaccgaacttaaactcagtgattatgagggacggctgattcagaacagcttaacaatagagagaatggtgctctctgcttttgacgagaggaggaataaatatctagaagaacatcccagtgcggggaaagatcctaagaaaactggaggacctatatacaggagagtagatggaaagtggaggagagaactcatcctttatgacaaagaagaaataagacgaatctggcgccaagctaataatggtgacgatgcaacggctggtctgactcacatgatgatctggcactccaatttgaatgatgcaacttaccagaggacaagagctcttgttcgcacaggaatggatcccaggatgtgctcactgatgcagggttcaaccctccctaggaggtctggggccgcaggtgctgcagtcaaaggagttggaacaatggtgatggaattgatcagaatgatcaaacgtgggatcaatgatcggaacttctggaggggtgagaatggacggagaacaaggattgcttatgaaagaatgtgcaacattctcaaagggaaatttcaaacagctgcacaaagaacaatggtggatcaagtgagagagagccggaatccaggaaatgctgagttcgaagatctcatctttttagcacggtctgcactcatattgagagggtcagttgctcacaagtcctgcctgcctgcctgtgtgtatggatctgccgtagccagtggatacgactttgaaagagagggatactctctagtcggaatagaccctttcagactgcttcaaaacagccaagtatacagcctaatcagaccaaatgagaatccagcacacaagagtcaactggtgtggatggcatgccattctgctgcatttgaagatctaagagtatcaagcttcatcagagggacgaaagtggtcccaagagggaagctttccactagaggagttcaaattgcttccaatgaaaacatggagactatggaatcaagtacccttgaactgagaagcagatactgggccataaggaccagaagtggagggaacaccaatcaacagagggcttcctcgggccaaatcagcatacaacctacgttctcagtacagagaaatctcccttttgacagaccaaccattatggcagcattcactgggaatacagaggggagaacatctgacatgagaaccgaaatcataaggctgatggaaagtgcaagaccagaagatgtgtctttccaggggcggggagtcttcgagctctcggacgaaaaggcaacgagcccgatcgtgccctcctttgacatgagtaatgaaggatcttatttcttcggagacaatgcagaggagtacgacaattaaagaaaaatacccttgtttctact

SEQ ID NO: 14 - protein, 498 aa, Artificial sequence
  Nucleoprotein NP of Influenza H1N1
MATKGTKRSYEQMETDGERQNATEIRASVGKMIDGIGRFYIQMCTELKLSDYEGRLIQNSLTIERMVLSAFDERRNKYLEEHPSAGKDPKKTGGPIYRRVDGKWRRELILYDKEEIRRIWRQANNGDDATAGLTHMMIWHSNLNDATYQRTRALVRTGMDPRMCSLMQGSTLPRRSGAAGAAVKGVGTMVMELIRMIKRGINDRNFWRGENGRRTRIAYERMCNILKGKFQTAAQRTMVDQVRESRNPGNAEFEDLIFLARSALILRGSVAHKSCLPACVYGSAVASGYDFEREGYSLVGIDPFRLLQNSQVYSLIRPNENPAHKSQLVWMACHSAAFEDLRVSSFIRGTKVVPRGKLSTRGVQIASNENMETMESSTLELRSRYWAIRTRSGGNTNQQRASSGQISIQPTFSVQRNLPFDRPTIMAAFTGNTEGRTSDMRTEIIRLMESARPEDVSFQGRGVFELSDEKATSPIVPSFDMSNEGSYFFGDNAEEYDN

SEQ ID NO: 15 - DNA, 1014 bp, Artificial sequence
  Matrix protein M of Influenza H1N1
agcaaaagcaggtagatattgaaagatgagtcttctaaccgaggtcgaaacgtacgttctctctatcgtcccgtcaggccccctcaaagccgagatcgcacagagacttgaagatgtctttgcagggaagaacaccgatcttgaggttctcatggaatggctaaagacaagaccaatcctgtcacctctgactaaggggattttaggatttgtgttcacgctcaccgtgcccagtgagcggggactgcagcgtagacgctttgtccaaaatgctcttaatgggaacggagatccaaataacatggacaaagcagttaaactgtataggaagcttaagagggagataacattccatggggccaaagaaatagcactcagttattctgctggtgcacttgccagttgtatgggcctcatatacaacaggatgggggctgtgaccactgaagtggcatttggcctggtatgcgcaacctgtgaacagattgctgactcccagcatcggtctcataggcaaatggtgacaacaaccaatccactaatcagacatgagaacagaatggttctagccagcactacagctaaggctatggagcaaatggctggatcgagtgagcaagcagcagaggccatggatattgctagtcaggccaggcaaatggtgcaggcgatgagaaccattgggactcatcctagctccagtgctggtctaaaagatgatcttcttgaaaatttgcaggcctatcagaaacgaatgggggtgcagatgcaacgattcaagtgatcctctcgtcattgcagcaaatatcattgagatcttgcacttgatattgtggattcttgatcgtctttttttcaaatgcatttatcgtcgctttaaatacggtttgaaaagagggccttctacggaaggagtgccagagtctatgagggaagaatatcgaaaggaacagcagaatgctgtggatgttgacgatggtcattttgtcaacatagagctggagtaaaaaacta

SEQ ID NO: 16 - protein, 252 aa, Artificial sequence
  Matrix protein M of Influenza H1N1
MSLLTEVETYVLSIVPSGPLKAEIAQRLEDVFAGKNTDLEVLMEWLKTRPILSPLTKGILGFVFTLTVPSERGLQRRRFVQNALNGNGDPNNMDKAVKLYRKLKREITFHGAKEIALSYSAGALASCMGLIYNRMGAVTTEVAFGLVCATCEQIADSQHRSHRQMVTTTNPLIRHENRMVLASTTAKAMEQMAGSSEQAAEAMDIASQARQMVQAMRTIGTHPSSSAGLKDDLLENLQAYQKRMGVQMQRFK

SEQ ID NO: 17 - DNA, 890 bp, Artificial sequence
  Non-structural protein 1 NS1 of Influenza H1N1
agcaaaagcagggtgacaaagacataatggatccaaacactgtgtcaagctttcaggtagattgctttctttggcatgtccgcaaaagagttgcagaccaagaactaggtgatgccccattccttgatcggcttcgccgagatcagaagtccctaagaggaagaggcagcactctcggtctggacatcgaaacagccacccgtgctggaaagcaaatagtggagcggattctgaaggaagaatctgatgaggcactcaaaatgaccatggcctctgtacctgcatcgcgctacctaactgacatgactcttgaggaaatgtcaaggcactggttcatgctcatgcccaagcagaaagtggcaggccctctttgtatcagaatggaccaggcgatcatggataagaacatcatactgaaagcgaacttcagtgtgatttttgaccggctggagactctaatattactaagggccttcaccgaagaggggacaattgttggcgaaatttcaccactgccctctcttccaggacatactgatgaggatgtcaaaaatgcagttggggtcctcatcggaggacttgaatggaataataacacagttcgagtctctgaaactctacagagattcgcttggagaagcagtaatgagaatgggagacctccactcactccaaaacagaaacggaaaatggcgggaacaattaggtcagaagtttgaagaaataagatggttgattgaagaagtgagacacagactgaagataacagagaatagttttgagcaaataacatttatgcaagccttacaactattgcttgaagtggagcaagagataagaactttctcgtttcagcttatttaataataaaaaacacccttgtttctact

SEQ ID NO: 18 - protein, 230 aa, Artificial sequence
  Non-structural protein 1 NS1 of Influenza H1N1
MDPNTVSSFQVDCFLWHVRKRVADQELGDAPFLDRLRRDQKSLRGRGSTLGLDIETATRAGKQIVERILKEESDEALKMTMASVPASRYLTDMTLEEMSRHWFMLMPKQKVAGPLCIRMDQAIMDKNIILKANFSVIFDRLETLILLRAFTEEGTIVGEISPLPSLPGHTDEDVKNAVGVLIGGLEWNNNTVRVSETLQRFAWRSSNENGRPPLTPKQKRKMAGTIRSEV

SEQ ID NO: 19 - DNA, 1070 bp, Artificial sequence
  influenza A virus-Headless HA withstalk domain and conservedglobular regions linked with linker (?HA/H1N1-I)
gaaaaacaaaagcaacaaaaatgaaggcaatactagtagttctgctatatacatttacaaccgcaaatgcagacacattatgtataggttatcatgcgaacaattcaacagacactgtagacacagtactagaaaagaatgtaacagtaacacactctgttaaccttctggaagacaagcataacggaaaactatgcggcggagggggctcatgcaatacaacttgtcagacacccgagggtgctataaacaccagcctcccatttcagaatatacatccgatcacaattggaaaatgtccaaagtatgtaaaaagcacaaaattgagactggccacaggattgaggaatgttccgtctattcaatctagaggcctattcggggccattgccggcttcattgaaggggggtggacagggatggtagatggatggtacggttatcaccatcaaaatgagcaggggtcaggatatgcagccgacctgaagagcacacaaaatgccattgacaagattactaacaaagtaaattctgttattgaaaagatgaatacacagttcacagcagtgggtaaagagttcaaccacctggaaaaaagaatagagaatctaaataaaaaagttgatgatggtttcctggacatttggacttacaatgccgaactgttggttctattggaaaatgaaagaactttggactatcacgattcaaatgtgaagaacttgtatgaaaaagtaagaaaccagttaaaaaacaatgccaaggaaattggaaacggctgctttgaattttaccacaaatgcgataacacgtgcatggaaagtgtcaaaaatgggacttatgactacccaaaatactcagaggaagcaaaattaaacagagaaaaaatagatggggtaaagctggaatcaacaaggatttaccagattttggcgatctattcaactgtcgccagttcattggtactggtagtctccctgggggcaatcagcttctggatgtgctctaatgggtctctacagtgtagaatatgtatttaaattaggatttcagaatcatgagaaaaacac

SEQ ID NO: 20 - protein, 339 aa, Artificial sequence
  influenza A virus-Headless HA withstalk domain and conservedglobular regions linked with linker (?HA/H1N1-I)
MKAILVVLLYTFTTANADTLCIGYHANNSTDTVDTVLEKNVTVTHSVNLLEDKHNGKLCGGGGSCNTTCQTPEGAINTSLPFQNIHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLESTRIYQILAIYSTVASSLVLVVSLGAISFWMCSNGSLQCRICI

SEQ ID NO: 21 - DNA, 1361 bp, Artificial sequence
  influenza A virus-Headless HA with stalk domain and more globular regions linked with linker (?HA/H1N1-II)
gaaaaacaaaagcaacaaaaatgaaggcaatactagtagttctgctatatacatttacaaccgcaaatgcagacacattatgtataggttatcatgcgaacaattcaacagacactgtagacacagtactagaaaagaatgtaacagtaacacactctgttaaccttctggaagacaagcataacggaaaactatgcaaactaagaggggtagccccattgcatttgggtaaatgtaacattgctggctggatcctgggaaatccagagtgtgaatcactctccacagcaagttcatggtcctacattgtggaaacatctaattcagacaatggaacgtgtggcggagggggctcaagaatgaactattactggacactagtagagccgggagacaaaataacattcgaagcaactggaaatctagtggtaccgagatatgcattcacaatggaaagaaatgctggatctggtattatcatttcagatacaccagtccacgattgcaatacaacttgtcagacacccgagggtgctataaacaccagcctcccatttcagaatatacatccgatcacaattggaaaatgtccaaagtatgtaaaaagcacaaaattgagactggccacaggattgaggaatgttccgtctattcaatctagaggcctattcggggccattgccggcttcattgaaggggggtggacagggatggtagatggatggtacggttatcaccatcaaaatgagcaggggtcaggatatgcagccgacctgaagagcacacaaaatgccattgacaagattactaacaaagtaaattctgttattgaaaagatgaatacacagttcacagcagtgggtaaagagttcaaccacctggaaaaaagaatagagaatctaaataaaaaagttgatgatggtttcctggacatttggacttacaatgccgaactgttggttctattggaaaatgaaagaactttggactatcacgattcaaatgtgaagaacttgtatgaaaaagtaagaaaccagttaaaaaacaatgccaaggaaattggaaacggctgctttgaattttaccacaaatgcgataacacgtgcatggaaagtgtcaaaaatgggacttatgactacccaaaatactcagaggaagcaaaattaaacagagaaaaaatagatggggtaaagctggaatcaacaaggatttaccagattttggcgatctattcaactgtcgccagttcattggtactggtagtctccctgggggcaatcagcttctggatgtgctctaatgggtctctacagtgtagaatatgtatttaaattaggatttcagaatcatgagaaaaacac

SEQ ID NO: 22 - protein, 436 aa, Artificial sequence
  influenza A virus-Headless HA with stalk domain and more globular regions linked with linker (?HA/H1N1-II)
MKAILVVLLYTFTTANADTLCIGYHANNSTDTVDTVLEKNVTVTHSVNLLEDKHNGKLCKLRGVAPLHLGKCNIAGWILGNPECESLSTASSWSYIVETSNSDNGTCGGGGSRMNYYWTLVEPGDKITFEATGNLVVPRYAFTMERNAGSGIIISDTPVHDCNTTCQTPEGAINTSLPFQNIHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLESTRIYQILAIYSTVASSLVLVVSLGAISFWMCSNGSLQCRICI

SEQ ID NO: 23 - DNA, 1346 bp, Artificial sequence
  influenza A virus-Headless HA with stalk domain and more globular regions linked without linker (?HA/H1N1-III)
gaaaaacaaaagcaacaaaaatgaaggcaatactagtagttctgctatatacatttacaaccgcaaatgcagacacattatgtataggttatcatgcgaacaattcaacagacactgtagacacagtactagaaaagaatgtaacagtaacacactctgttaaccttctggaagacaagcataacggaaaactatgcaaactaagaggggtagccccattgcatttgggtaaatgtaacattgctggctggatcctgggaaatccagagtgtgaatcactctccacagcaagttcatggtcctacattgtggaaacatctaattcagacaatggaacgtgtagaatgaactattactggacactagtagagccgggagacaaaataacattcgaagcaactggaaatctagtggtaccgagatatgcattcacaatggaaagaaatgctggatctggtattatcatttcagatacaccagtccacgattgcaatacaacttgtcagacacccgagggtgctataaacaccagcctcccatttcagaatatacatccgatcacaattggaaaatgtccaaagtatgtaaaaagcacaaaattgagactggccacaggattgaggaatgttccgtctattcaatctagaggcctattcggggccattgccggcttcattgaaggggggtggacagggatggtagatggatggtacggttatcaccatcaaaatgagcaggggtcaggatatgcagccgacctgaagagcacacaaaatgccattgacaagattactaacaaagtaaattctgttattgaaaagatgaatacacagttcacagcagtgggtaaagagttcaaccacctggaaaaaagaatagagaatctaaataaaaaagttgatgatggtttcctggacatttggacttacaatgccgaactgttggttctattggaaaatgaaagaactttggactatcacgattcaaatgtgaagaacttgtatgaaaaagtaagaaaccagttaaaaaacaatgccaaggaaattggaaacggctgctttgaattttaccacaaatgcgataacacgtgcatggaaagtgtcaaaaatgggacttatgactacccaaaatactcagaggaagcaaaattaaacagagaaaaaatagatggggtaaagctggaatcaacaaggatttaccagattttggcgatctattcaactgtcgccagttcattggtactggtagtctccctgggggcaatcagcttctggatgtgctctaatgggtctctacagtgtagaatatgtatttaaattaggatttcagaatcatgagaaaaacac

SEQ ID NO: 24 - protein, 431 aa, Artificial sequence
  influenza A virus-Headless HA with stalk domain and more globular regions linked without linker (?HA/H1N1-III)
MKAILVVLLYTFTTANADTLCIGYHANNSTDTVDTVLEKNVTVTHSVNLLEDKHNGKLCKLRGVAPLHLGKCNIAGWILGNPECESLSTASSWSYIVETSNSDNGTCRMNYYWTLVEPGDKITFEATGNLVVPRYAFTMERNAGSGIIISDTPVHDCNTTCQTPEGAINTSLPFQNIHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLESTRIYQILAIYSTVASSLVLVVSLGAISFWMCSNGSLQCRICI

SEQ ID NO: 25 - DNA, 1080 bp, Artificial sequence
  influenza A virus-Headless HA withstalk domain and conservedglobular regions linked with linker (?HA/H3N2-I)
ggataattctattaaccatgaagactatcattgctttgagctgcattctatgtctggttttcgctcaaaaaattcctggaaatgacaatagcacggcaacgctgtgccttgggcaccatgcagtaccaaacggaacgatagtgaaaacaatcacgaatgaccgaattgaagttactaatgctactgagctggttcagaactcctcaataggtgaaatatgcggcggagggggctcatgcaagtctgaatgcatcactccaaatggaagcattccaaatgacaaaccattccaaaatgtaaacaggatcacatacggggcatgtcccagatatgttaagcaaagcactctgaaattggcaacaggaatgcgaaatgtaccagagagacaaactagaggcatatttggcgcaatagcgggtttcatagaaaatggttgggagggaatggtggatggttggtacggcttcaggcatcaaaattctgagggaagaggacaagcagcagatcttaaaagcactcaagcagcaatcgatcaaatcaatgggaagctgaatcgattgatcgggaaaaccaacgagaaattccatcagattgaaaaagagttctcagaagtagaagggagaattcaggaccttgagaaatatgttgaggacacaaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcatggggtcaatcagaaatggaacttatgaccacaatgtatacagggatgaagcattaaacaaccggttccagatcaagggagttgagctgaagtcagggtacaaagattggatcctatggatttcctttgccatatcatgttttttgctttgtgttgctctgttggggttcatcatgtgggcctgccaaaagggcaacattaggtgcaacatttgcatttgagtgcattaattaaaaacac

SEQ ID NO: 26 - protein, 347 aa, Artificial sequence
  influenza A virus-Headless HA withstalk domain and conservedglobular regions linked with linker (?HA/H3N2-I)
MKTIIALSCILCLVFAQKIPGNDNSTATLCLGHHAVPNGTIVKTITNDRIEVTNATELVQNSSIGEICGGGGSCKSECITPNGSIPNDKPFQNVNRITYGACPRYVKQSTLKLATGMRNVPERQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACMGSIRNGTYDHNVYRDEALNNRFQIKGVELKSGYKDWILWISFAISCFLLCVALLGFIMWACQKGNIRCNICI

SEQ ID NO: 27 - DNA, 1359 bp, Artificial sequence
  influenza A virus-Headless HA with stalk domain and more globular regions linked with linker (?HA/H3N2-II)
ggataattctattaaccatgaagactatcattgctttgagctgcattctatgtctggttttcgctcaaaaaattcctggaaatgacaatagcacggcaacgctgtgccttgggcaccatgcagtaccaaacggaacgatagtgaaaacaatcacgaatgaccgaattgaagttactaatgctactgagctggttcagaactcctcaataggtgaaatatgcgacagtcctcatcagatccttgatggagaaaactgcacactaatagatgctctattgggagaccctcagtgtgatggctttcaaaataagaaatgggaccttttcgttgaacgaaacaaagcctacagcaactgtggcggagggggctcaagaataagcatctattggacaatagtaaaaccaggagacatacttttgattaacagcacagggaatctaattgctcctaggggttacttcaaaatacgaagtgggaaaagctcaataatgagatcagatgcacccattggcaagtgcaagtctgaatgcatcactccaaatggaagcattccaaatgacaaaccattccaaaatgtaaacaggatcacatacggggcatgtcccagatatgttaagcaaagcactctgaaattggcaacaggaatgcgaaatgtaccagagagacaaactagaggcatatttggcgcaatagcgggtttcatagaaaatggttgggagggaatggtggatggttggtacggcttcaggcatcaaaattctgagggaagaggacaagcagcagatcttaaaagcactcaagcagcaatcgatcaaatcaatgggaagctgaatcgattgatcgggaaaaccaacgagaaattccatcagattgaaaaagagttctcagaagtagaagggagaattcaggaccttgagaaatatgttgaggacacaaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcatggggtcaatcagaaatggaacttatgaccacaatgtatacagggatgaagcattaaacaaccggttccagatcaagggagttgagctgaagtcagggtacaaagattggatcctatggatttcctttgccatatcatgttttttgctttgtgttgctctgttggggttcatcatgtgggcctgccaaaagggcaacattaggtgcaacatttgcatttgagtgcattaattaaaaacac

SEQ ID NO: 28 - protein, 440 aa, Artificial sequence
  influenza A virus  Other feature Headless HA with stalk domain and more globular regions linked with linker (?HA/H3N2-II)
MKTIIALSCILCLVFAQKIPGNDNSTATLCLGHHAVPNGTIVKTITNDRIEVTNATELVQNSSIGEICDSPHQILDGENCTLIDALLGDPQCDGFQNKKWDLFVERNKAYSNCGGGGSRISIYWTIVKPGDILLINSTGNLIAPRGYFKIRSGKSSIMRSDAPIGKCKSECITPNGSIPNDKPFQNVNRITYGACPRYVKQSTLKLATGMRNVPERQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACMGSIRNGTYDHNVYRDEALNNRFQIKGVELKSGYKDWILWISFAISCFLLCVALLGFIMWACQKGNIRCNICI

SEQ ID NO: 29 - DNA, 1344 bp, Artificial sequence
  influenza A virus-Headless HA with stalk domain and more globular regions linked without linker (?HA/H3N2-III)
ggataattctattaaccatgaagactatcattgctttgagctgcattctatgtctggttttcgctcaaaaaattcctggaaatgacaatagcacggcaacgctgtgccttgggcaccatgcagtaccaaacggaacgatagtgaaaacaatcacgaatgaccgaattgaagttactaatgctactgagctggttcagaactcctcaataggtgaaatatgcgacagtcctcatcagatccttgatggagaaaactgcacactaatagatgctctattgggagaccctcagtgtgatggctttcaaaataagaaatgggaccttttcgttgaacgaaacaaagcctacagcaactgtagaataagcatctattggacaatagtaaaaccaggagacatacttttgattaacagcacagggaatctaattgctcctaggggttacttcaaaatacgaagtgggaaaagctcaataatgagatcagatgcacccattggcaagtgcaagtctgaatgcatcactccaaatggaagcattccaaatgacaaaccattccaaaatgtaaacaggatcacatacggggcatgtcccagatatgttaagcaaagcactctgaaattggcaacaggaatgcgaaatgtaccagagagacaaactagaggcatatttggcgcaatagcgggtttcatagaaaatggttgggagggaatggtggatggttggtacggcttcaggcatcaaaattctgagggaagaggacaagcagcagatcttaaaagcactcaagcagcaatcgatcaaatcaatgggaagctgaatcgattgatcgggaaaaccaacgagaaattccatcagattgaaaaagagttctcagaagtagaagggagaattcaggaccttgagaaatatgttgaggacacaaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcatggggtcaatcagaaatggaacttatgaccacaatgtatacagggatgaagcattaaacaaccggttccagatcaagggagttgagctgaagtcagggtacaaagattggatcctatggatttcctttgccatatcatgttttttgctttgtgttgctctgttggggttcatcatgtgggcctgccaaaagggcaacattaggtgcaacatttgcatttgagtgcattaattaaaaacac

SEQ ID NO: 30 - protein, 435 aa, Artificial sequence
  influenza A virus-Headless HA with stalk domain and more globular regions linked without linker (?HA/H3N2-III)
MKTIIALSCILCLVFAQKIPGNDNSTATLCLGHHAVPNGTIVKTITNDRIEVTNATELVQNSSIGEICDSPHQILDGENCTLIDALLGDPQCDGFQNKKWDLFVERNKAYSNCRISIYWTIVKPGDILLINSTGNLIAPRGYFKIRSGKSSIMRSDAPIGKCKSECITPNGSIPNDKPFQNVNRITYGACPRYVKQSTLKLATGMRNVPERQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACMGSIRNGTYDHNVYRDEALNNRFQIKGVELKSGYKDWILWISFAISCFLLCVALLGFIMWACQKGNIRCNICI

SEQ ID NO: 31 - DNA, 1388 bp, Artificial sequence
  influenza A/WSN/33 virus-Headless HA with stalk domain and more globular regions linked with linker (?HA/WSN-II)
agcaaaagcaggggaaaataaaaacaaccaaaatgaaggcaaaactaggcggagggggctcactggtcctgttatatgcatttgtagctacagatgcagacacaatatgtataggctaccatgcgaacaactcaaccgacactgttgacacaatattcgagaagaatgtggcagtgacacattctgttaacctgctcgaagacagacacaacgggaaactatgtaaattaaaaggaatagccccactacaattggggaaatgtaacatcaccggatggctcttgggaaatccagaatgcgactcactgcttccagcgagatcatggtcctacattgtagaaacaccaaactctgagaatggagcatgtaggatgaactattactggaccttgctagaacccggagacacaataatatttgaggcaactggtaatctaatagcaccatggtatgctttcgcactgagtagagggtttgagtccggcatcatcacctcaaacgcgtcaatgcatgagtgtaacacgaagtgtcaaacaccccagggatctataaacagcaatctccctttccagaatatacacccagtcacaataggagagtgcccaaaatatgtcaggagtaccaaattgaggatggttacaggactaagaaacatcccatccattcaatacagaggtctatttggagccattgctggttttattgaggggggatggactggaatgatagatggatggtatggttatcatcatcagaatgaacagggatcaggctatgcagcggatcaaaaaagcacacagaatgccattaacgggattacaaacaaggtgaactctgttatcgagaaaatgaacactcaattcacagctgtgggtaaagaattcaacaacttagaaaaaaggatggaaaatttaaataaaaaagttgatgatgggtttctggacatttggacatataatgcagaattgttagttctactggaaaatgaaagaactttggatttccatgacttaaatgtgaagaatctgtacgagaaagtaaaaagccaattaaagaataatgccaaagaaatcggaaatgggtgttttgagttctaccacaagtgtgacaatgaatgcatggaaagtgtaagaaatgggacttatgattatccaaaatattcagaagaatcaaagttgaacagggaaaagatagatggagtgaaattggaatcaatgggggtgtatcagattctggcgatctactcaactgtcgccagttcactggtgcttttggtctccctgggggcaatcagtttctggatgtgttctaatgggtctttgcagtgcagaatatgcatctgagattagaatttcagagatatgaggaaaaacacccttgtttctact

SEQ ID NO: 32 - protein, 436 aa, Artificial sequence
  influenza A/WSN/33 virus-Headless HA with stalk domain and more globular regions linked with linker (?HA/WSN-II)
MKAKLLVLLYAFVATDADTICIGYHANNSTDTVDTIFEKNVAVTHSVNLLEDRHNGKLCKLKGIAPLQLGKCNITGWLLGNPECDSLLPARSWSYIVETPNSENGACGGGGSRMNYYWTLLEPGDTIIFEATGNLIAPWYAFALSRGFESGIITSNASMHECNTKCQTPQGSINSNLPFQNIHPVTIGECPKYVRSTKLRMVTGLRNIPSIQYRGLFGAIAGFIEGGWTGMIDGWYGYHHQNEQGSGYAADQKSTQNAINGITNKVNSVIEKMNTQFTAVGKEFNNLEKRMENLNKKVDDGFLDIWTYNAELLVLLENERTLDFHDLNVKNLYEKVKSQLKNNAKEIGNGCFEFYHKCDNECMESVRNGTYDYPKYSEESKLNREKIDGVKLESMGVYQILAIYSTVASSLVLLVSLGAISFWMCSNGSLQCRICI

SEQ ID NO: 33 - DNA, 1373 bp, Artificial sequence
  influenza A/WSN/33 virus-Headless HA with stalk domain and more globular regions linked without linker (?HA/WSN-III)
agcaaaagcaggggaaaataaaaacaaccaaaatgaaggcaaaactactggtcctgttatatgcatttgtagctacagatgcagacacaatatgtataggctaccatgcgaacaactcaaccgacactgttgacacaatattcgagaagaatgtggcagtgacacattctgttaacctgctcgaagacagacacaacgggaaactatgtaaattaaaaggaatagccccactacaattggggaaatgtaacatcaccggatggctcttgggaaatccagaatgcgactcactgcttccagcgagatcatggtcctacattgtagaaacaccaaactctgagaatggagcatgtaggatgaactattactggaccttgctagaacccggagacacaataatatttgaggcaactggtaatctaatagcaccatggtatgctttcgcactgagtagagggtttgagtccggcatcatcacctcaaacgcgtcaatgcatgagtgtaacacgaagtgtcaaacaccccagggatctataaacagcaatctccctttccagaatatacacccagtcacaataggagagtgcccaaaatatgtcaggagtaccaaattgaggatggttacaggactaagaaacatcccatccattcaatacagaggtctatttggagccattgctggttttattgaggggggatggactggaatgatagatggatggtatggttatcatcatcagaatgaacagggatcaggctatgcagcggatcaaaaaagcacacagaatgccattaacgggattacaaacaaggtgaactctgttatcgagaaaatgaacactcaattcacagctgtgggtaaagaattcaacaacttagaaaaaaggatggaaaatttaaataaaaaagttgatgatgggtttctggacatttggacatataatgcagaattgttagttctactggaaaatgaaagaactttggatttccatgacttaaatgtgaagaatctgtacgagaaagtaaaaagccaattaaagaataatgccaaagaaatcggaaatgggtgttttgagttctaccacaagtgtgacaatgaatgcatggaaagtgtaagaaatgggacttatgattatccaaaatattcagaagaatcaaagttgaacagggaaaagatagatggagtgaaattggaatcaatgggggtgtatcagattctggcgatctactcaactgtcgccagttcactggtgcttttggtctccctgggggcaatcagtttctggatgtgttctaatgggtctttgcagtgcagaatatgcatctgagattagaatttcagagatatgaggaaaaacacccttgtttctact

SEQ ID NO: 34 - protein, 431 aa, Artificial sequence
  influenza A/WSN/33 virus-Headless HA with stalk domain and more globular regions linked without linker (?HA/WSN-III)
MKAKLLVLLYAFVATDADTICIGYHANNSTDTVDTIFEKNVAVTHSVNLLEDRHNGKLCKLKGIAPLQLGKCNITGWLLGNPECDSLLPARSWSYIVETPNSENGACRMNYYWTLLEPGDTIIFEATGNLIAPWYAFALSRGFESGIITSNASMHECNTKCQTPQGSINSNLPFQNIHPVTIGECPKYVRSTKLRMVTGLRNIPSIQYRGLFGAIAGFIEGGWTGMIDGWYGYHHQNEQGSGYAADQKSTQNAINGITNKVNSVIEKMNTQFTAVGKEFNNLEKRMENLNKKVDDGFLDIWTYNAELLVLLENERTLDFHDLNVKNLYEKVKSQLKNNAKEIGNGCFEFYHKCDNECMESVRNGTYDYPKYSEESKLNREKIDGVKLESMGVYQILAIYSTVASSLVLLVSLGAISFWMCSNGSLQCRICI

SEQ ID NO: 35 - DNA, 1701 bp, Artificial sequence
  influenza A/Michigan/45/2015 virus-Complete HA
atgaaggcaatactagtagttctgctatatacatttacaaccgcaaatgcagacacattatgtataggttatcatgcgaacaattcaacagacactgtagacacagtactagaaaagaatgtaacagtaacacactctgttaaccttctggaagacaagcataacggaaaactatgcaaactaagaggggtagccccattgcatttgggtaaatgtaacattgctggctggatcctgggaaatccagagtgtgaatcactctccacagcaagttcatggtcctacattgtggaaacatctaattcagacaatggaacgtgttacccaggagatttcatcaattatgaggagctaagagagcaattgagctcagtgtcatcatttgaaaggtttgagatattccccaagacaagttcatggcccaatcatgactcgaacaaaggtgtaacggcagcatgtcctcacgctggagcaaaaagcttctacaaaaacttgatatggctagttaaaaaaggaaattcatacccaaagcttaaccaatcctacattaatgataaagggaaagaagtcctcgtgctgtggggcattcaccatccatctactactgctgaccaacaaagtctctatcagaatgcagatgcatatgtttttgtggggacatcaagatacagcaagaagttcaagccggaaatagcaacaagacccaaagtgagggatcgagaagggagaatgaactattactggacactagtagagccgggagacaaaataacattcgaagcaactggaaatctagtggtaccgagatatgcattcacaatggaaagaaatgctggatctggtattatcatttcagatacaccagtccacgattgcaatacaacttgtcagacacccgagggtgctataaacaccagcctcccatttcagaatatacatccgatcacaattggaaaatgtccaaagtatgtaaaaagcacaaaattgagactggccacaggattgaggaatgttccgtctattcaatctagaggcctattcggggccattgccggcttcattgaaggggggtggacagggatggtagatggatggtacggttatcaccatcaaaatgagcaggggtcaggatatgcagccgacctgaagagcacacaaaatgccattgacaagattactaacaaagtaaattctgttattgaaaagatgaatacacagttcacagcagtgggtaaagagttcaaccacctggaaaaaagaatagagaatctaaataaaaaagttgatgatggtttcctggacatttggacttacaatgccgaactgttggttctattggaaaatgaaagaactttggactatcacgattcaaatgtgaagaacttgtatgaaaaagtaagaaaccagttaaaaaacaatgccaaggaaattggaaacggctgctttgaattttaccacaaatgcgataacacgtgcatggaaagtgtcaaaaatgggacttatgactacccaaaatactcagaggaagcaaaattaaacagagaaaaaatagatggggtaaagctggaatcaacaaggatttaccagattttggcgatctattcaactgtcgccagttcattggtactggtagtctccctgggggcaatcagcttctggatgtgctctaatgggtctctacagtgtagaatatgtatttaa

SEQ ID NO: 36 - protein, 566 aa, Artificial sequence
  influenza A/Michigan/45/2015virus-Complete HA
MKAILVVLLYTFTTANADTLCIGYHANNSTDTVDTVLEKNVTVTHSVNLLEDKHNGKLCKLRGVAPLHLGKCNIAGWILGNPECESLSTASSWSYIVETSNSDNGTCYPGDFINYEELREQLSSVSSFERFEIFPKTSSWPNHDSNKGVTAACPHAGAKSFYKNLIWLVKKGNSYPKLNQSYINDKGKEVLVLWGIHHPSTTADQQSLYQNADAYVFVGTSRYSKKFKPEIATRPKVRDREGRMNYYWTLVEPGDKITFEATGNLVVPRYAFTMERNAGSGIIISDTPVHDCNTTCQTPEGAINTSLPFQNIHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLESTRIYQILAIYSTVASSLVLVVSLGAISFWMCSNGSLQCRICI

SEQ ID NO: 37 - DNA, 1701 bp, Artificial sequence
  influenza A/Kansas/14/2017 virus-Complete HA
atgaagactatcattgctttgagctgcattctatgtctggttttcgctcaaaaaattcctggaaatgacaatagcacggcaacgctgtgccttgggcaccatgcagtaccaaacggaacgatagtgaaaacaatcacgaatgaccgaattgaagttactaatgctactgagctggttcagaactcctcaataggtgaaatatgcgacagtcctcatcagatccttgatggagaaaactgcacactaatagatgctctattgggagaccctcagtgtgatggctttcaaaataagaaatgggaccttttcgttgaacgaaacaaagcctacagcaactgttacccttatgatgtgccggattatgcatcccttagatcactagttgcctcatccggcacactggagtttaacaatgaaagcttcaattgggctggagtcactcaaaacggaacaagttcttcttgcataaggggatctaaaagtagtttctttagtagattaaattggttgacccacttaaactccaaatacccagcattaaacgtgactatgccaaacaatgaacaatttgacaaattgtacatttggggtgttcaccacccgggtacggacaaggaccaaatctccctgtatgcacaatcatcaggaagaatcacagtatctaccaaaagaagccaacaagctgtaatcccgaatatcggatctagacccagaataagggatatccctagcagaataagcatctattggacaatagtaaaaccaggagacatacttttgattaacagcacagggaatctaattgctcctaggggttacttcaaaatacgaagtgggaaaagctcaataatgagatcagatgcacccattggcaagtgcaagtctgaatgcatcactccaaatggaagcattccaaatgacaaaccattccaaaatgtaaacaggatcacatacggggcatgtcccagatatgttaagcaaagcactctgaaattggcaacaggaatgcgaaatgtaccagagagacaaactagaggcatatttggcgcaatagcgggtttcatagaaaatggttgggagggaatggtggatggttggtacggcttcaggcatcaaaattctgagggaagaggacaagcagcagatcttaaaagcactcaagcagcaatcgatcaaatcaatgggaagctgaatcgattgatcgggaaaaccaacgagaaattccatcagattgaaaaagagttctcagaagtagaagggagaattcaggaccttgagaaatatgttgaggacacaaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcatggggtcaatcagaaatggaacttatgaccacaatgtatacagggatgaagcattaaacaaccggttccagatcaagggagttgagctgaagtcagggtacaaagattggatcctatggatttcctttgccatatcatgttttttgctttgtgttgctctgttggggttcatcatgtgggcctgccaaaagggcaacattaggtgcaacatttgcatttga

SEQ ID NO: 38 - protein, 566 aa, Artificial sequence
  influenza A/Kansas/14/2017 virus-Complete HA
MKTIIALSCILCLVFAQKIPGNDNSTATLCLGHHAVPNGTIVKTITNDRIEVTNATELVQNSSIGEICDSPHQILDGENCTLIDALLGDPQCDGFQNKKWDLFVERNKAYSNCYPYDVPDYASLRSLVASSGTLEFNNESFNWAGVTQNGTSSSCIRGSKSSFFSRLNWLTHLNSKYPALNVTMPNNEQFDKLYIWGVHHPGTDKDQISLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPERQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACMGSIRNGTYDHNVYRDEALNNRFQIKGVELKSGYKDWILWISFAISCFLLCVALLGFIMWACQKGNIRCNICI

SEQ ID NO: 39 - DNA, 1433 bp, Artificial sequence
  Influenza A/Michigan/45/2015 virus- Neuraminidase
agtttaaaatgaatccaaaccaaaagataataaccattggttcgatctgtatgacaattggaatggctaacttaatattacaaattggaaacataatctcaatatgggttagccactcaattcaaattggaaatcaaagccagattgaaacatgcaatcaaagcgtcattacttatgaaaacaacacttgggtaaatcagacatatgttaacatcagcaacaccaactttgctgctggacagtcagtggtttccgtgaaattagcgggcaattcctctctctgccctgttagtggatgggctatatacagtaaagacaacagtgtaagaatcggttccaagggggatgtgtttgtcataagggaaccattcatatcatgctctcccttggaatgcagaaccttcttcttgactcaaggggccttgctaaatgacaaacattccaatggaaccattaaagacaggagcccataccgaaccctaatgagctgtcctattggtgaagttccctctccatacaactcaagatttgagtcagtcgcttggtcagcaagtgcttgtcatgatggcatcaattggctaacaattggaatttctggcccagacagtggggcagtggctgtgttaaagtacaatggcataataacagacactatcaagagttggaggaacaatatattgagaacacaagagtctgaatgtgcatgtgtaaatggttcttgctttaccataatgaccgatggaccaagtgatggacaggcctcatacaaaatcttcagaatagaaaagggaaagataatcaaatcagtcgaaatgaaagcccctaattatcactatgaggaatgctcctgttaccctgattctagtgaaatcacatgtgtgtgcagggataactggcatggctcgaatcgaccgtgggtgtctttcaaccagaatctggaatatcagatgggatacatatgcagtggggttttcggagacaatccacgccctaatgataagacaggcagttgtggtccagtatcgtctaatggagcaaatggagtaaaaggattttcattcaaatacggcaatggtgtttggatagggagaactaaaagcattagttcaagaaaaggttttgagatgatttgggatccgaatggatggactgggactgacaataaattctcaataaagcaagatatcgtaggaataaatgagtggtcagggtatagcgggagttttgttcagcatccagaactaacagggctggattgtataagaccttgcttctgggttgaactaataagagggcgacccgaagagaacacaatctggactagcgggagcagcatatccttttgtggtgtaaacagtgacactgtgggttggtcttggccagacggtgctgagttgccatttaccattgacaagtaatttgttcaaaaaact

SEQ ID NO: 40 - protein, 469 aa, Artificial sequence
  influenza A/Michigan/45/2015 virus Other featureNeuraminidase
MNPNQKIITIGSICMTIGMANLILQIGNIISIWVSHSIQIGNQSQIETCNQSVITYENNTWVNQTYVNISNTNFAAGQSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 41 - DNA, 1441 bp, Artificial sequence
  influenza A/Kansas/14/2017 virus Other feature Neuraminidase
agtaaagatgaatccaaatcaaaagataataacgattggctctgtttctctcaccatttccacaatatgcttcttcatgcaaattgccatcctgataactactgtaacattgcatttcaagcaatatgaattcaactcccccccaaacaaccaagtgatgctgtgtgaaccaacaataatagaaagaaacataacagagatagtgtatttgaccaacaccaccatagagagggaaatatgccccaaaccagcagaatacagaaattggtcaaaaccgcaatgtggcattacaggatttgcacctttctctaaggacaattcgattaggctttccgctggtggggacatctgggtgacaagagaaccttatgtgtcatgcgatcctgacaagtgttatcaatttgcccttggacagggaacaacaataaacaacgtgcattcaaataacacagcacgtgataggacccctcatcggactctattgatgaatgagttgggtgttcctttccatctggggaccaagcaagtgtgcatagcatggtccagctcaagttgtcacgatggaaaagcatggctgcatgtttgtataacgggggatgataaaaatgcaactgctagtttcatttacaatgggaggcttgtagatagtgttgtttcatggtccaaagatattctcaggacccaggagtcagaatgcgtttgtatcaatggaacttgtacagtagtaatgactgatggaaatgctacaggaaaagctgatactaaaatattattcattgaggaggggaaaatcgttcatactagcaaattgtcaggaagtgctcagcatgtcgaagagtgctcttgctatcctcgataccctggtgtcagatgtgtctgcagagacaactggaaaggatccaaccggcccatcgtagatataaacataaaggatcatagcattgtttccagttatgtgtgttcaggacttgttggagacacacccagaaaaaccgacagctccagcagcagccattgcttgaatcctaacaatgaaaaaggtggtcatggagtgaaaggctgggcctttgatgatggaaatgacgtgtggatggggagaacaatcaacgagacgtcacgcttagggtatgaaaccttcaaagtcgttgaaggctggtccaaccctaagtccaaattgcagataaataggcaagtcatagttgacagaggtgataggtccggttattctggtattttctctgttgaaggcaaaagctgcatcaatcggtgcttttatgtggagttgattaggggaagaaaagaggaaactgaagtcttgtggacctcaaacagtattgttgtgttttgtggcacctcaggtacatatggaacaggctcatggcctgatggggcggacctcaatctcatgcatatataagctttcgcaattttagaaaaaact

SEQ ID NO: 42 - protein, 469 aa, Artificial sequence
  influenza A/Kansas/14/2017 virusOther feature Neuraminidase
MNPNQKIITIGSVSLTISTICFFMQIAILITTVTLHFKQYEFNSPPNNQVMLCEPTIIERNITEIVYLTNTTIEREICPKPAEYRNWSKPQCGITGFAPFSKDNSIRLSAGGDIWVTREPYVSCDPDKCYQFALGQGTTINNVHSNNTARDRTPHRTLLMNELGVPFHLGTKQVCIAWSSSSCHDGKAWLHVCITGDDKNATASFIYNGRLVDSVVSWSKDILRTQESECVCINGTCTVVMTDGNATGKADTKILFIEEGKIVHTSKLSGSAQHVEECSCYPRYPGVRCVCRDNWKGSNRPIVDINIKDHSIVSSYVCSGLVGDTPRKTDSSSSSHCLNPNNEKGGHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGADLNLMHI

SEQ ID NO: 43 - DNA, 2341 bp, Artificial sequence
  influenza A/PR/8/34 virus Other feature Polymerase basic protein 1 (PB1)
agcgaaagcaggcaaaccatttgaatggatgtcaatccgaccttacttttcttaaaagtgccaacacaaaatgctataagcacaactttcccttatactggagaccctccttacagccatgggacaggaacaggatacaccatggatactgtcaacaggacacatcagtactcagaaaagggaagatggacaacaaacaccgaaactggagcaccgcaactcaacccgattgatgggccactgccagaagacaatgaaccaagtggttatgcccaaacagattgtgtattggaggcgatggctttccttgaggaatcccatcctggtatttttgaaaactcgtgtattgaaacgatggaggttgttcagcaaacacgagtagacaagctgacacaaggccgacagacctatgactggactctaaatagaaaccaacctgctgcaacagcattggccaacacaatagaagtgttcagatcaaatggcctcacggccaatgagtctggaaggctcatagacttccttaaggatgtaatggagtcaatgaacaaagaagaaatggggatcacaactcattttcagagaaagagacgggtgagagacaatatgactaagaaaatgataacacagagaacaatgggtaaaaagaagcagagattgaacaaaaggagttatctaattagagcattgaccctgaacacaatgaccaaagatgctgagagagggaagctaaaacggagagcaattgcaaccccagggatgcaaataagggggtttgtatactttgttgagacactggcaaggagtatatgtgagaaacttgaacaatcagggttgccagttggaggcaatgagaagaaagcaaagttggcaaatgttgtaaggaagatgatgaccaattctcaggacaccgaactttctttcaccatcactggagataacaccaaatggaacgaaaatcagaatcctcggatgtttttggccatgatcacatatatgaccagaaatcagcccgaatggttcagaaatgttctaagtattgctccaataatgttctcaaacaaaatggcgagactgggaaaagggtatatgtttgagagcaagagtatgaaacttagaactcaaatacctgcagaaatgctagcaagcatcgatttgaaatatttcaatgattcaacaagaaagaagattgaaaaaatccgaccgctcttaatagaggggactgcatcattgagccctggaatgatgatgggcatgttcaatatgttaagcactgtattaggcgtctccatcctgaatcttggacaaaagagatacaccaagactacttactggtgggatggtcttcaatcctctgacgattttgctctgattgtgaatgcacccaatcatgaagggattcaagccggagtcgacaggttttatcgaacctgtaagctacttggaatcaatatgagcaagaaaaagtcttacataaacagaacaggtacatttgaattcacaagttttttctatcgttatgggtttgttgccaatttcagcatggagcttcccagttttggggtgtctgggatcaacgagtcagcggacatgagtattggagttactgtcatcaaaaacaatatgataaacaatgatcttggtccagcaacagctcaaatggcccttcagttgttcatcaaagattacaggtacacgtaccgatgccatagaggtgacacacaaatacaaacccgaagatcatttgaaataaagaaactgtgggagcaaacccgttccaaagctggactgctggtctccgacggaggcccaaatttatacaacattagaaatctccacattcctgaagtctgcctaaaatgggaattgatggatgaggattaccaggggcgtttatgcaacccactgaacccatttgtcagccataaagaaattgaatcaatgaacaatgcagtgatgatgccagcacatggtccagccaaaaacatggagtatgatgctgttgcaacaacacactcctggatccccaaaagaaatcgatccatcttgaatacaagtcaaagaggagtacttgaggatgaacaaatgtaccaaaggtgctgcaatttatttgaaaaattcttccccagcagttcatacagaagaccagtcgggatatccagtatggtggaggctatggtttccagagcccgaattgatgcacggattgatttcgaatctggaaggataaagaaagaagagttcactgagatcatgaagatctgttccaccattgaagagctcagacggcaaaaatagtgaatttagcttgtccttcatgaaaaaatgccttgtttctact

SEQ ID NO: 44 - protein, 757 aa, Artificial sequence
  influenza A/PR/8/34 virus Other feature Polymerase basic protein 1 (PB1)
MDVNPTLLFLKVPAQNAISTTFPYTGDPPYSHGTGTGYTMDTVNRTHQYSEKGRWTTNTETGAPQLNPIDGPLPEDNEPSGYAQTDCVLEAMAFLEESHPGIFENSCIETMEVVQQTRVDKLTQGRQTYDWTLNRNQPAATALANTIEVFRSNGLTANESGRLIDFLKDVMESMNKEEMGITTHFQRKRRVRDNMTKKMITQRTMGKKKQRLNKRSYLIRALTLNTMTKDAERGKLKRRAIATPGMQIRGFVYFVETLARSICEKLEQSGLPVGGNEKKAKLANVVRKMMTNSQDTELSFTITGDNTKWNENQNPRMFLAMITYMTRNQPEWFRNVLSIAPIMFSNKMARLGKGYMFESKSMKLRTQIPAEMLASIDLKYFNDSTRKKIEKIRSLLIEGTASLSPGMMMGMFNMLSTVLGVSILNLGQKRYTKTTYWWDGLQSSDDFALIVNAPNHEGIQAGVDRFYRTCKLLGINMSKKKSYINRTGTFEFTSFFYRYGFVANFSMELPSFGVSGINESADMSIGVTVIKNNMINNDLGPATAQMALQLFIKDYRYTYRCHRGDTQIQTRRSFEIKKLWEQTRSKAGLLVSDGGPNLYNIRNLHIPEVCLKWELMDEDYQGRLCNPLNPFVSHKEIESMNNAVMMPAHGPAKNMEYDAVATTHSWIPKRNRSILNTSQRGVLEDEQMYQRCCNLFEKFFPSSSYRRPVGISSMVEAMVSRARIDARIDFESGRIKKEEFTEIMKICSTIEELRRQK

SEQ ID NO: 45 - DNA, 2341 bp, Artificial sequence
  influenza A/PR/8/34 virus Other feature Polymerase basic protein 2 (PB2)
agcgaaagcaggtcaattatattcaatatggaaagaataaaagaactaagaaatctaatgtcgcagtctcgcacccgcgagatactcacaaaaaccaccgtggaccatatggccataatcaagaagtacacatcaggaagacaggagaagaacccagcacttaggatgaaatggatgatggcaatgaaatatccaattacagcagacaagaggataacggaaatgattcctgagagaaatgagcaaggacaaactttatggagtaaaatgaatgatgccggatcagaccgagtgatggtatcacctctggctgtgacatggtggaataggaatggaccaatgacaaatacagttcattatccaaaaatctacaaaacttattttgaaagagtcgaaaggctaaagcatggaacctttggccctgtccattttagaaaccaagtcaaaatacgtcggagagttgacataaatcctggtcatgcagatctcagtgccaaggaggcacaggatgtaatcatggaagttgttttccctaacgaagtgggagccaggatactaacatcggaatcgcaactaacgataaccaaagagaagaaagaagaactccaggattgcaaaatttctcctttgatggttgcatacatgttggagagagaactggtccgcaaaacgagattcctcccagtggctggtggaacaagcagtgtgtacattgaagtgttgcatttgactcaaggaacatgctgggaacagatgtatactccaggaggggaagtgaagaatgatgatgttgatcaaagcttgattattgctgctaggaacatagtgagaagagctgcagtatcagcagacccactagcatctttattggagatgtgccacagcacacagattggtggaattaggatggtagacatccttaagcagaacccaacagaagagcaagccgtgggtatatgcaaggctgcaatgggactgagaattagctcatccttcagttttggtggattcacatttaagagaacaagcggatcatcagtcaagagagaggaagaggtgcttacgggcaatcttcaaacattgaagataagagtgcatgagggatatgaagagttcacaatggttgggagaagagcaacagccatactcagaaaagcaaccaggagattgattcagctgatagtgagtgggagagacgaacagtcgattgccgaagcaataattgtggccatggtattttcacaagaggattgtatgataaaagcagttagaggtgatctgaatttcgtcaatagggcgaatcagcgactgaatcctatgcatcaacttttaagacattttcagaaggatgcgaaagtgctttttcaaaattggggagttgaacctatcgacaatgtgatgggaatgattgggatattgcccgacatgactccaagcatcgagatgtcaatgagaggagtgagaatcagcaaaatgggtgtagatgagtactccagcacggagagggtagtggtgagcattgaccggttcttgagagtccgggaccaacgaggaaatgtactactgtctcccgaggaggtcagtgaaacacagggaacagagaaactgacaataacttactcatcgtcaatgatgtgggagattaatggtcctgaatcagtgttggtcaatacctatcaatggatcatcagaaactgggaaactgttaaaattcagtggtcccagaaccctacaatgctatacaataaaatggaatttgaaccatttcagtctttagtacctaaggccattagaggccaatacagtgggtttgtgagaactctgttccaacaaatgagggatgtgcttgggacatttgataccgcacagataataaaacttcttcccttcgcagccgctccaccaaagcaaagtagaatgcagttctcctcatttactgtgaatgtgaggggatcaggaatgagaatacttgtaaggggcaattctcctgtattcaactacaacaaggccacgaagagactcacagttctcggaaaggatgctggcactttaaccgaagacccagatgaaggcacagctggagtggagtccgctgttctgaggggattcctcattctgggcaaagaagacaggagatatgggccagcattaagcatcaatgaactgagcaaccttgcgaaaggagagaaggctaatgtgctaattgggcaaggagacgtggtgttggtaatgaaacgaaaacgggactctagcatacttactgacagccagacagcgaccaaaagaattcggatggccatcaattagtgtcgaatagtttaaaaacgaccttgtttctact

SEQ ID NO: 46 - protein, 759 aa, Artificial sequence
  influenza A/PR/8/34 virus Other feature Polymerase basic protein 2 (PB2)
MERIKELRNLMSQSRTREILTKTTVDHMAIIKKYTSGRQEKNPALRMKWMMAMKYPITADKRITEMIPERNEQGQTLWSKMNDAGSDRVMVSPLAVTWWNRNGPITNTVHYPKIYKTYFERVERLKHGTFGPVHFRNQVKIRRRVDINPGHADLSAKEAQDVIMEVVFPNEVGARILTSESQLTITKEKKEELQDCKISPLMVAYMLERELVRKTRFLPVAGGTSSVYIEVLHLTQGTCWEQMYTPGGEVRNDDVDQSLIIAARNIVRRAAVSADPLASLLEMCHSTQIGGIRMVDILRQNPTEEQAVDICKAAMGLRISSSFSFGGFTFKRTSGSSVKREEEVLTGNLQTLKIRVHEGYEEFTMVGRRATAILRKATRRLIQLIVSGRDEQSIAEAIIVAMVFSQEDCMIKAVRGDLNFVNRANQRLNPMHQLLRHFQKDAKVLFQNWGVEPIDNVMGMIGILPDMTPSIEMSMRGVRISKMGVDEYSSTERVVVSIDRFLRIRDQRGNVLLSPEEVSETQGTEKLTITYSSSMMWEINGPESVLVNTYQWIIRNWETVKIQWSQNPTMLYNKMEFEPFQSLVPKAIRGQYSGFVRTLFQQMRDVLGTFDTAQIIKLLPFAAAPPKQSRMQFSSFTVNVRGSGMRILVRGNSPVFNYNKATKRLTVLGKDAGTLTEDPDEGTAGVESAVLRGFLILGKENKRYGPALSINELSNLAKGEKANVLIGQGDVVLVMKRKRDSSILTDSQTATKRIRMAIN

SEQ ID NO: 47 - DNA, 2233 bp, Artificial sequence
  influenza A/PR/8/34 virus Other feature polymerase acid protein (PA)
agcgaaagcaggtactgatccaaaatggaagattttgtgcgacaatgcttcaatccgatgattgtcgagcttgcggaaaaaacaatgaaagagtatggggaggacctgaaaatcgaaacaaacaaatttgcagcaatatgcactcacttggaagtatgcttcatgtattcagatttccacttcatcaatgagcaaggcgagtcaataatcgtagaacttggtgatcctaatgcacttttgaagcacagatttgaaataatcgagggaagagatcgcacaatggcctggacagtagtaaacagtatttgcaacactacaggggctgagaaaccaaagtttctaccagatttgtatgattacaaggaaaatagattcatcgaaattggagtaacaaggagagaagttcacatatactatctggaaaaggccaataaaattaaatctgagaaaacacacatccacattttctcgttcactggggaagaaatggccacaaaggccgactacactctcgatgaagaaagcagggctaggatcaaaaccaggctattcaccataagacaagaaatggccagcagaggcctctgggattcctttcgtcagtccgagagaggagaagagacaattgaagaaaggtttgaaatcacaggaacaatgcgcaagcttgccgaccaaagtctcccgccgaacttctccagccttgaaaattttagagcctatgtggatggattcgaaccgaacggctacattgagggcaagctgtctcaaatgtccaaagaagtaaatgctagaattgaaccttttttgaaaacaacaccacgaccacttagacttccgaatgggcctccctgttctcagcggtccaaattcctgctgatggatgccttaaaattaagcattgaggacccaagtcatgaaggagagggaataccgctatatgatgcaatcaaatgcatgagaacattctttggatggaaggaacccaatgttgttaaaccacacgaaaagggaataaatccaaattatcttctgtcatggaagcaagtactggcagaactgcaggacattgagaatgaggagaaaattccaaagactaaaaatatgaaaaaaacaagtcagctaaagtgggcacttggtgagaacatggcaccagaaaaggtagactttgacgactgtaaagatgtaggtgatttgaagcaatatgatagtgatgaaccagaattgaggtcgcttgcaagttggattcagaatgagttcaacaaggcatgcgaactgacagattcaagctggatagagcttgatgagattggagaagatgtggctccaattgaacacattgcaagcatgagaaggaattatttcacatcagaggtgtctcactgcagagccacagaatacataatgaagggggtgtacatcaatactgccttacttaatgcatcttgtgcagcaatggatgatttccaattaattccaatgataagcaagtgtagaactaaggagggaaggcgaaagaccaacttgtatggtttcatcataaaaggaagatcccacttaaggaatgacaccgacgtggtaaactttgtgagcatggagttttctctcactgacccaagacttgaaccacacaaatgggagaagtactgtgttcttgagataggagatatgcttctaagaagtgccataggccaggtttcaaggcccatgttcttgtatgtgaggacaaatggaacctcaaaaattaaaatgaaatggggaatggagatgaggcgttgtctcctccagtcacttcaacaaattgagagtatgattgaagctgagtcctctgtcaaagagaaagacatgaccaaagagttctttgagaacaaatcagaaacatggcccattggagagtctcccaaaggagtggaggaaagttccattgggaaggtctgcaggactttattagcaaagtcggtatttaacagcttgtatgcatctccacaactagaaggattttcagctgaatcaagaaaactgcttcttatcgttcaggctcttagggacaatctggaacctgggacctttgatcttggggggctatatgaagcaattgaggagtgcctaattaatgatccctgggttttgcttaatgcttcttggttcaactccttccttacacatgcattgagttagttgtggcagtgctactatttgctatccatactgtccaaaaaagtaccttgtttctact

SEQ ID NO: 48 - protein, 716 aa, Artificial sequence
  influenza A/PR/8/34 virus Other feature polymerase acid protein (PA)
MEEFVRQCFNPMIVELAEKTMKEYGEDPKIETNKFAAICTHLEVCFMYSDFHFIDERGESKIVESGDPNALLKHRFEIIEGRDRTMAWTVVNSICNTTGVEKPKFLPDLYDYKENRFIEIGVTRREVHIYYLEKANKIKSEKTHIHIFSFTGEEMATKADYTLDEESRARIKTRLFTIRQEMASRGLWDSFRQSERGEETIEERFEITGTMRRLAEQSLPPNFSSLENFRAYVDGFKPNGCIEGKLSQMSKEVNARIEPFLKTTPRPLRLPDGPPCSQRSKFLLMDALKLSIEDPSHEGEGIPLHDAIKCMKTFFGWKEPNIVKPHEKGINPNYLLAWNQVLAELKDIENEEKIPKTKNMKKTSQLKWALGENMAPEKVDFEDCKDISDLKQYDSDEPEQRSLASWIQSEFNKACELTDSGWIELDEIGEDVAPIEHIASMRRNYFTAEVSHCRATEYIMKGVYINTALLNASCAAMDDFQLIPMISKCRTKEGRRKTNLYGFIIKGRSHLRNDTDVVNFVSMEFSLTDPRLEPHKWEKYCVLEIGDMLLRTAIGQVSRPMFLYVRTNGTSKIKMKWGMEMRRCLLQSLQQVESMVEAESSVKEKDMTKEFFENKSKTWPIGESPKGVEEGSIGKVCRTLLAKSVFNSLYASPQLEGFSAESRKLLLIVQALRDNLEPGTFDLGGLYGAIEECLINDPWVLLNASWFNSFLTHALK

SEQ ID NO: 49 - DNA, 1540 bp, Artificial sequence
  influenza A/PR/8/34 virus Other feature Nucleoprotein (NP)
gtagataatcactcactgagtgacatcaaaatcatggcgtcccaaggcaccaaacggtcttacgaacagatggagactgatggagaacgccagaatgccactgaaatcagagcatccgtcggaaaaatgattggtggaattggacgattctacatccaaatgtgcaccgaactcaaactcagtgattatgagggacggttgatccaaaacagcttaacaatagagagaatggtgctctctgcttttgacgaaaggagaaataaatacctggaagaacatcccagtgcggggaaagatcctaagaaaactggaggacctatatacaggagagtaaacggaaagtggatgagagaactcatcctttatgacaaagaagaaataaggcgaatctggcgccaagctaataatggtgacgatgcaacggctggtctgactcacatgatgatctggcattccaatttgaatgatgcaacttatcagaggacaagagctcttgttcgcaccggaatggatcccaggatgtgctctctgatgcaaggttcaactctccctaggaggtctggagccgcaggtgctgcagtcaaaggagttggaacaatggtgatggaattggtcaggatgatcaaacgtgggatcaatgatcggaacttctggaggggtgagaatggacgaaaaacaagaattgcttatgaaagaatgtgcaacattctcaaagggaaatttcaaactgctgcacaaaaagcaatgatggatcaagtgagagagagccggaacccagggaatgctgagttcgaagatctcacttttctagcacggtctgcactcatattgagagggtcggttgctcacaagtcctgcctgcctgcctgtgtgtatggacctgccgtagccagtgggtacgactttgaaagagagggatactctctagtcggaatagaccctttcagactgcttcaaaacagccaagtgtacagcctaatcagaccaaatgagaatccagcacacaagagtcaactggtgtggatggcatgccattctgccgcatttgaagatctaagagtattaagcttcatcaaagggacgaaggtgctcccaagagggaagctttccactagaggagttcaaattgcttccaatgaaaatatggagactatggaatcaagtacacttgaactgagaagcaggtactgggccataaggaccagaagtggaggaaacaccaatcaacagagggcatctgcgggccaaatcagcatacaacctacgttctcagtacagagaaatctcccttttgacagaacaaccattatggcagcattcaatgggaatacagagggaagaacatctgacatgaggaccgaaatcataaggatgatggaaagtgcaagaccagaagatgtgtctttccaggggcggggagtcttcgagctctcggacgaaaaggcagcgagcccgatcgtgccttcctttgacatgagtaatgaaggatcttatttcttcggagacaatgcagaggagtacgacaattaaagaaaaatac

SEQ ID NO: 50 - protein, 498 aa, Artificial sequence
  influenza A/PR/8/34 virus Other feature Nucleoprotein (NP)
MASQGTKRSYEQMETDGERQNATEIRASVGKMIGGIGRFYIQMCTELKLSDYEGRLIQNSLTIERMVLSAFDERRNKYLEEHPSAGKDPKKTGGPIYRRVNGKWMRELILYDKEEIRRIWRQANNGDDATAGLTNMMIWHSNLNDATYQRTRALVRTGMDPRMCSLMQGSTLPRRSGAAGAAVKGVGTMVMELVRMIKRGINDRNFWRGENGRKTRIAYERMCNILKGKFQTAAQKAMMDQVRESRDPGNAEFEDLTFLARSALILRGSVAHKSCLPACVYGPAVASGYDFEREGYSLVGIDPFRLLQNSQVYSLIRPNENPAHKSQLVWMACHSAAFEDLRVLSFIKGTKVVPRGKLSTRGVQIASNENMETMESSTLELRSRYWAIRTRSGGNTNQQRASAGQISIQPTFSVQRNLPFDRTTVMAAFTGNTEGRTSDMRTEIIRMMESARPEDVSFQGRGVFELSDEKAASPIVPSFDMSNEGSYFFGDNAEEYDN

SEQ ID NO: 51 - DNA, 1027 bp, Artificial sequence
  influenza A/PR/8/34 virus Other featureMatrix protein (M)
agcgaaagcaggtagatattgaaagatgagtcttctaaccgaggtcgaaacgtacgttctctctatcatcccgtcaggccccctcaaagccgagatcgcacagagacttgaagatgtctttgcagggaagaacaccgatcttgaggttctcatggaatggctaaagacaagaccaatcctgtcacctctgactaaggggattttaggatttgtgttcacgctcaccgtgcccagtgagcgaggactgcagcgtagacgctttgtccaaaatgcccttaatgggaacggggatccaaataacatggacaaagcagttaaactgtataggaagctcaagagggagataacattccatggggccaaagaaatctcactcagttattctgctggtgcacttgccagttgtatgggcctcatatacaacaggatgggggctgtgaccactgaagtggcatttggcctggtatgtgcaacctgtgaacagattgctgactcccagcatcggtctcataggcaaatggtgacaacaaccaacccactaatcagacatgagaacagaatggttttagccagcactacagctaaggctatggagcaaatggctggatcgagtgagcaagcagcagaggccatggaggttgctagtcaggctaggcaaatggtgcaagcgatgagaaccattgggactcatcctagctccagtgctggtctgaaaaatgatcttcttgaaaatttgcaggcctatcagaaacgaatgggggtgcagatgcaacggttcaagtgatcctctcgctattgccgcaaatatcattgggatcttgcacttgatattgtggattcttgatcgtctttttttcaaatgcatttaccgtcgctttaaatacggactgaaaggagggccttctacggaaggagtgccaaagtctatgagggaagaatatcgaaaggaacagcagagtgctgtggatgctgacgatggtcattttgtcagcatagagctggagtaaaaaactaccttgtttctact

SEQ ID NO: 52 - protein, 252 aa, Artificial sequence
  influenza A/PR/8/34 virus Other featureMatrix protein (M)
MSLLTEVETYVLSIIPSGPLKAEIAQRLEDVFAGKNTDLEALMEWLKTRPILSPLTKGILGFVFTLTVPSERGLQRRRFVQNALNGNGDPNNMDKAVKLYRKLKREITFHGAKEISLSYSAGALASCMGLIYNRMGAVTTEVAFGLVCATCEQIADSQHRSHRQMVTTTNPLIRHENRMVLASTTAKAMEQMAGSSEQAAEAMEVASQARQMVQAMRTIGTHPSSSAGLKNDLLENLQAYQKRMGVQMQRFK

SEQ ID NO: 53 - DNA, 890 bp, Artificial sequence
  influenza A/PR/8/34 virus Other featurenon- structural protein (NS)
agcaaaagcagggtgacaaaaacataatggatccaaacactgtgtcaagctttcaggtagattgctttctttggcatgtccgcaaacgagttgcagaccaagaactaggtgatgccccattccttgatcggcttcgccgagatcagaaatccctaagaggaaggggcagcaccctcggtctggacatcgagacagccacacgtgctggaaagcagatagtggagcggattctgaaagaagaatccgatgaggcacttaaaatgaccatggcctctgtacctgcgtcgcgttacctaactgacatgactcttgaggaaatgtcaagggactggtccatgctcatacccaagcagaaagtggcaggccctctttgtatcagaatggaccaggcgatcatggataagaacatcatactgaaagcgaacttcagtgtgaattttgaccggctggagactctaatattgctaagggctttcaccgaagagggagcaattgttggcgaaatttcaccattgccttctcttccaggacatactgctgaggatgtcaaaaatgcagttggagtcctcatcgggggacttgaatggaatgataacacagttcgagtctctgaaactctacagagattcgcttggagaagcagtaatgagaatgggagacctccactcactccaaaacagaaacgagaaatggcgggaacaattaggtcagaagtttgaagaaataagatggttgattgaagaagtgagacacaaactgaagataacagagaatagttttgagcaaataacatttatgcaagccttacatctattgcttgaagtggagcaagagataagaactttctcgtttcagcttatttagtaataaaaaacacccttgtttctact

SEQ ID NO: 54 - protein, 237 aa, Artificial sequence
  influenza A/PR/8/34 virus Other featurenon- structural protein (NS)
MDSNTVSSFQVDCFLWHVRKQVVDQELGDAPFLDRLRRDQKSLRGRGSTLGLNIEAATRVGKQIVERILKEESDEALRMTMASAPASRYLTDMTIEELSRDWFMLMPKQKVEGPLCIRIDQAIMDKNIMLKANFSVIFDRLETLILLRAFTEEEAIVGEISPLPSLPGHTIEDVKNAVGVLIGGLEWNDNTVRVSKNLQRFAWRSSNENGRHPLTPKQKREMAGTIRSKVRRDKMAD